(19) United States
(12) Patent Application Publication
(10) Pub. No.: US 2019/0328783 A1
(43) Pub. Date: Oct. 31, 2019

(54) A METHOD OF ENGINEERING PRODRUG-SPECIFIC HYPERSENSITIVE T-CELLS FOR IMMUNOTHERAPY BY GENE EXPRESSION

(71) Applicant: CELLECTIS, Paris (FR)

(72) Inventors: Julien VALTON, NEW YORK, NY (US); Philippe DUCHATEAU, DRAVEIL (FR); Laurent POIROT, PARIS (FR); Barbra Johnsson SASU, SAN FRANCISCO, CA (US); Arvind RAJPAL, SAN FRANCISCO, CA (US)

(21) Appl. No.: 16/092,414

(22) PCT Filed: Apr. 13, 2017
(86) PCT No.: PCT/EP2017/058923

§ 371 Date: Oct. 9, 2018

(30) Foreign Application Priority Data
Apr. 15, 2016 (DK) PA 201670233

(51) Int. Cl. A61K 35/17; C07K 14/725; C12N 5/0783; C12N 15/85

(52) U.S. Cl. CPC A61K 35/17; C07K 14/7051; C12N 5/0636; A61K 45/06; C12N 2510/00; C12N 2015/8518; C12Y 305/04005; C12N 15/85

(57) ABSTRACT

The present invention relates to therapeutic cells for immunotherapy to treat patients with cancer. In particular, the inventors develop a method of engineering prodrug-specific hypersensitive T-cell, which can be depleted in vivo by the administration of said specific prodrug in case of occurrence of a serious adverse event. The invention opens the way to safer and tunable adoptive immunotherapy strategies for treating cancer.

### FIELD OF THE INVENTION

The present invention relates to the use of therapeutic cells for cell therapy or immunotherapy to treat patients with various pathologies such as cancer, infection or autoimmune disease. In particular, the invention provides with a method of engineering human cells, preferably immune cells, such as T cells, to make them hypersensitive to a specific drug, in particular approved drugs, to be administrated to the patient to safely deplete such engineered cells, so as to modulate or terminate cell therapy treatment”. The invention opens the way to safer tunable adoptive immunotherapy strategies, especially for treating cancer.

### BACKGROUND OF THE INVENTION

Adoptive immunotherapy, which involves the transfer of autologous antigen-specific immune cells generated ex vivo, is a promising strategy to treat cancer. For instance, the T-cells used for adoptive immunotherapy can be generated either by expansion of antigen-specific T cells or redirection of T-cells through genetic engineering (Park, Rosenberg et al. 2011). Transfer of viral antigen specific immune cells is a well-established procedure used for the treatment of transplant associated viral infections and rare viral-related malignancies. Similarly, isolation and transfer of tumor specific T-cells has been shown to be successful in treating melanoma. Novel specificities in T-cells have been successfully generated through the genetic transfer of transgenic T cell receptors or chimeric antigen receptors (CARs). CARs are synthetic receptors consisting of a targeting moiety that is associated with one or more signaling domains in a single fusion molecule. CARs have successfully allowed T-cells to be redirected against antigens expressed at the surface of tumor cells from various malignancies including lymphomas and solid tumors (Jena, Dotti et al. 2010).

Immune cell adoptive immunotherapy which can involve the transfer of antigen-specific T-cells generated ex-vivo, is a promising strategy to treat cancer. T-cells used for adoptive immunotherapy can be generated through the genetic transfer of transgenic T cell receptors or chimeric antigen receptors (CARs). CARs are synthetic receptors consisting of a targeting moiety that is associated with one or more signaling domains. CARs have successfully allowed T-cells to be redirected against antigens expressed at the surface of tumor cells from various malignancies including lymphomas and solid tumors. However, despite their unprecedent efficacy for tumor eradication in vivo, CAR T cells can promote acute adverse events after being transferred into patients. Among the potential adverse events is Graft versus host disease (GvHD), on-target off-tumor activity or aberrant lymphoproliferative capacity due to vector derived insertional mutagenesis. Therefore, there is still a need to modulate the immune response induced by the engineered cells and to develop cell specific depletion systems to adjust treatments and prevent such deleterious events to occur in vivo. One way to deplete CAR T cell would be to endow them with hypersensitivity properties toward a specific prodrug. The inventors have sought for one particular depletion system based on prodrug hypersensitivity.

In order to address these problems, the inventors have found that one way to control immune cells would be to endow them with hypersensitivity properties toward a specific chemical-based prodrug compound, preferably an already approved drug. In particular, they found that the expression of some specific genes directly or indirectly involved in the compound metabolization and toxicity target/pathways can be successfully obtained to confer drug sensitivity to immune cells. They also found that this hypersensitivity could be induced in combination with the engineering of the same cells to confer resistance to other drugs. Accordingly, therapeutic cells can be made sensitive to approved drugs and also made resistant to chemotherapy or immune suppressive treatments for their use in combination therapy.

### SUMMARY OF THE INVENTION

In a general aspect, the present invention provides methods of producing human cell, preferably immune cell, and more particularly T-cells, that may be depleted in-vivo as part of a cell or immuno-therapy treatment, said method comprising a step of induction of a prodrug hypersensitivity into said cell by selectively overexpressing at least one endogenous gene or expressing a transgene involved in the activation of said prodrug.

In one embodiment, such endogenous gene or transgene may be CDA, which codes for cytidine deaminase, which expression renders the engineered human cell, preferably immune cell hypersensitive to 5-formyl-2′-deoxycytidine (5fdC) or 5-hydroxymethyl-2′-deoxycytidine (5hmdC).

In another embodiment, such endogenous gene or transgene codes for cytochromes P450, such as, more specifically, CYP2D6-1, CYP2D6-2, CYP2C9, CYP3A4, CYP2C19 orCYP1A2, which have been found to make the engineered human cells, preferably immune cells, of the present invention, hypersensitive to cyclophosphamide and/or isophosphamide. Such expression was particularly efficient when the transgenes were introduced into the cells by viral transduction, in particular by using lentiviral vectors.

Further engineering of the human cells according to the present invention, could be obtained such as by inactivating the expression of endogeneous gene(s), with the effect of conferring either resistance or hypersensitivity to other drugs, in particular approved drugs.

The prodrug-hypersensitive immune cell according to the invention, such asT-cells or NK cells, are usually further engineered to express a Chimeric Antigen Receptor (CAR) that confers to the cells more specificity towards pathological cell types. It may also be of a great advantage to engineering such cells to make them less alloreactive by inactivating the expression of the genes encoding T cell receptors subunits such as TCRalpha or TCRbeta and to enhance their immune activity by inactivating the expression immune-checkpoint gene(s) in these cells.

The present invention finally provides with isolated engineered human cells or populations of engineered human cells obtainable by the methods of the present invention, preferably immune cells, rendered sensitive to a prodrug, aspharmaceutical compositions for use in the treatment of cancer, infection or immune disease. Such cells can be especially used together with or in sequential combination with at least one prodrug to which said cell has been made hypersensitive, for a safer immunotherapy treatment.

### BRIEF DESCRIPTION OF THE FIGURES

FIG. 1: Analysis by FACS for BFP expression in T cells render hypersensitive to the prodrugs 5fdC and 5HmdC after CDA expression. mRNA encoding a chimeric construction consisting of CDA fused to a BFP reporter. A) Viability rate expressed in percentage: comparison between mock (T cells not transfected by the CDA expression plasmid)—represented in the graph by unfilled bar- and transfected T cells by the CDA expression plasmid—represented in the graph by filled bar-, when all cells are submitted to an increasing dose of the 5fdC prodrug (from 0 to 10 mM); B) The same than for A) excepted that the cells are submitted to an increasing dose of the 5HmdC prodrug (from 0 to 10 mM).

FIG. 2: Analysis by FACS for BFP expression in T cells render hypersensitive to the prodrugs 5fdC and 5HmdC by combining CDA expression and inactivation of dCK gene by KO. mRNA encoding a chimeric construction consisting of CDA fused to a BFP reporter, and a KO inactivation of dCK is made by using TALE-nuclease as explained later. A) Viability rate expressed in percentage: comparison between mock (T cells which have undergone KO on dCK and not transfected by the CDA expression plasmid)—represented in the graph by unfilled bar- and KO dCK T cells transfected by the CDA expression plasmid—represented in the graph by filled bar-, when all cells are submitted to an increasing dose of the 5fdC prodrug (from 0 to 10 mM); B) The same than for A) excepted that the cells are submitted to an increasing dose of the 5HmdC prodrug (from 0 to 10 mM).

FIG. 3: Analysis by FACS for testing viability of engineered T cell in presence of clofarabine. A comparison is made between KO dCK T cells (unfilled bar) vs T cells having undergone KO dCK T cells and a CDA expression (dark filled bar) vs WT T cells (clear filled bar) in presence of increasing doses of clofarabine (from 0 to 100 μM).

FIG. 4: Schematic representation of the different single chain chimeric antigen receptor (scCAR) Architecture (V1 to V6) as preferred ones which can be used within the scope of the present invention.

### DESCRIPTION OF THE INVENTION

Unless specifically defined herein, all technical and scientific terms used have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, N.Y.: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

Method of Engineering (Pro)Drug Sensitive Human Cells for Depletion Purpose

The inventors has found that drug hypersensitivity could be applied to human cells, in particular immune cells, to provide some sort of “switch off” system in case of occurrence of adverse event, following the administration of said engineered cells to a patient. This situation contrasts with prior art (ex: (Pavlos R et al, 2015, Annu Rev Med; 66: 439-454) which disclose that prodrugs T cells hypersensitivity, such as Type 4 hypersensitivity often called delayed type hypersensitivity (HS), can be considered as being associated with a type of adverse drug reaction (ADR), thus as unwanted reactions.

The inventors provide in the scope of the present invention with a method of producing human cells, preferably immune cells, for a safer cancer therapy, by providing the means to deplete engineered said cells, in case of occurrence of adverse event. This is achieved by conferring drug hypersensitivity to said cells by expressing specific gene(s) involved in the toxicity of a given prodrug to a cell, making this prodrug active in said cell by, for instance, chemical conversion, metabolization, lack of excretion or detoxification of the active drug. This activation of the prodrug into an active drug thereby allows the depletion of the engineered cells of the invention in-vivo.

According to a preferred aspect of the invention, immune cells, such as CAR-T cells are made sensitive to a prodrug, prior to being administrated to a patient, so that said prodrug can be administered to said patient later on to terminate or modulate cell therapy treatment (ex. occurrence of an adverse event).

Accordingly, the present invention relates to a method of producing human cell that may be depleted in-vivo as part of a cell therapy or immunotherapy treatment, comprising one or several of the following steps:

(a) Providing a human cell;

(b) Inducing drug hypersensitivity into said cell by selectively expressing at least one transgene or overexpressing at least an endogenous gene involved in the mechanism of action of said drug making this drug, initially referred to as “prodrug”, becoming toxic to said cell,

(c) Optionally assaying the hypersensitivity of said cell engineered in step b) to said drug;

(d) Expanding said engineered cell obtained in step b).

In a preferred embodiment, the present invention refers to a method of producing] human cell, preferably immune cell that may be depleted in-vivo as part of cell therapy or an immunotherapy treatment, said method comprising one or several of the following steps:

(a) Providing an immune cell;

(b) Inducing a prodrug hypersensitivity into said human cell, preferably immune cell by selectively overexpressing an endogenous gene and/or a transgene involved in the toxicity of a prodrug to such cell

(c) Optionally assaying the hypersensitivity to said prodrug of the human cell, preferably immune cell engineered in step b);

(d) Expanding the engineered cells obtained in step b).

By “involved in the toxicity of a prodrug”, it is meant that the selective expression of a transgene or the selective overexpression of at least one endogenous gene is involved in the specific conversion of said prodrug to drug which is toxic to said immune cell.

For sake of simplification, the term “overexpression” is used herein for designating both the expression of a transgene or the overexpression of a gene that is endogenous to the cell and which expression is normally (i.e. in established culture conditions) not sufficient in non-engineered cells to make them sensitive to the prodrug.

The term “prodrug” designates a molecule the cell is normally resistant to (i.e. not sensitive to), when this molecule is provided into the cell medium at a given concentration. This “prodrug” becomes a “drug” if its IC50 in the cell medium is lowered, preferably by 20%, more preferably by 50% and even more preferably by 70% upon engineering of the cells according to the invention. By “in vivo depletion of human cell”, it is meant in the present invention that the depletion may be complete, almost complete or partial. The level of depletion depends of the therapeutic goal to achieve. By “complete in vivo depletion”—i.e 100% of the cells are depleted—applies particularly when engineered human cells—mainly immune cells—of the invention are found harmful against host cells (such as in a graft-versus-host event). A less stringent in vivo depletion of engineered cells may be performed to deplete more than 95% of engineered human cells of the present invention administrated to the patient. This almost complete depletion may be applied in case of an adverse event such a cytokine release storm (CRS) in which activated engineered immune cells administrated to the patient release cytokines, producing a type of systemic inflammatory response. Finally, a partial in depletion may be applied—at least of 50%—, in case a modulation of the response of the engineered human cells, preferably immune cells, is sought. This modulation can be useful, for instance, to restrain the activity of CAR-T cells, when those have been found overaggressive (ie limit “off targets”). The depletion of prodrug hypersensitive immune cells may be detected for example by using the methods described in the examples herein or by any other suitable method known in the art (i.e FACs cytometry).

This in vivo depletion is particularly adapted and required when a serious adverse event happens. Such adverse event may occur in case of allogeneic bone marrow transplantation when T cells were recognized as the central mediators of graft-versus-host disease (GVHD) or Cytokine release syndrome (CRS). Although the antigenic targets in adoptive T cell therapy are much better defined, the potential for adverse effects, both on-target and off-target, remains. Finally, other side events may be elevated liver enzymes, acute pulmonary infiltrates or B-cell depletion or hypogamma-globulinemia.

The doses of prodrug to be used for depleting prodrug-hypersensitive engineered immune cells of the present invention have a value inferior or equal to those for which the Cmax is obtained, in order to minimize the probability of adverse events.

According to a preferred embodiment, said in vivo depletion of human cells made drug-specific hypersensitive is performed to an extent that at least 50%, preferably 95% or more preferably 100% of such cells are depleted.

Preferably, human cells to be depleted are human immune cells, preferably T cells, and more preferably CD8+ T cells are destroyed following the action of the specific drug being administrated to the patient. The depletion of drug hypersensitive immune cells may be detected for example by using the methods described in the examples herein or by any other suitable method known in the art.

By “transgene”, it is meant a nucleic acid sequence introduced into the cell (encoding one or more polypeptides), which can be exogenous to the cell or be an additional modified or unmodified copy of a sequence already present in the genome of the cell.

Said transgene usually encodes a product, generally an enzyme, involved in the mechanism of action of the drug, such as an enzyme which is implicated in the prodrug metabolic pathway. enzyme that may be selected in a non-limitative group comprising hydrolase, reductase, oxidase, transferase, esterase, dehydrogenase, peroxidase, kinase, tautomerase, deaminase, dehydratase. The transgene can be designed to be inserted, or can be inserted, into the cell genome in such a way as to alter the genome of the cell into which it is inserted (e.g. it is inserted at a location which differs from that of the natural gene or its insertion results in a knockout). A transgene can include one or more transcriptional regulatory sequences and any other nucleic acid, such as introns, that may be necessary for optimal expression of the selected nucleic acid encoding polypeptide. The polypeptide encoded by the transgene is preferably expressed under a biologically active form in cells in which the transgene is inserted. By “gene” is meant the basic unit of heredity, consisting of a segment of DNA arranged in a linear manner along a chromosome, which codes for a specific protein or segment of protein, small RNA and the like. A gene typically includes a promoter, a 5′ untranslated region, one or more coding sequences (exons), optionally introns, a 3′ untranslated region. The gene may further comprise a terminator, enhancers and/or silencers.

By “inducing a drug hypersensitivity into said cell”, it is meant that after being engineered by expression of at least one prodrug-related gene, the cell is enable to metabolize, degrade or detoxify said prodrug after being genetically engineered in order to express the suitable enzyme. Thus, an amount of the corresponding drug, preferably a prodrug, is becoming cytotoxic to said engineered cell. The expression “specific-prodrug hypersensitive human cell” corresponds to the human cell, preferably immune cell, which is able to express or overexpress at least one enzyme delivered in said cell, said enzyme being implicated in the conversion of the prodrug to drug. Thus, an amount of the corresponding drug—which is generally inferior to the dose to get the Cmax—is becoming cytotoxic to said engineered human cell and therefore allows for their depletion.

By the terms “selectively expressing”, it is intended that the human cell, preferably immune cell in which an additional gene is introduced is enabled to produce the polypeptide encoded by said additional gene, said cell not expressing generally said protein at a significant level. In particular, this is the case of most P450 cytochrome family genes (i.e. CYP3A4, CYP2C9, CYP2C19) which exist in the genome of immune cells but are not expressed in native immune cell (i.e. non-engineered) or at a much lower level—generally at least 50%, preferably at least 75%, more preferably at least 100% and even more preferably 200% lower than the expression level observed into the engineered immune cell in the same experimental or treatment conditions. Said engineered cell is therefore enabled to produce the specific functional enzyme necessary for the conversion of the prodrug to the drug which is toxic to said immune cell

By the terms “selectively overexpressing”, it is intended that the non-engineered human cell, preferably immune cell is already producing the polypeptide and that by introducing an additional gene, said cell is enabled to produce at least 50%, preferably at least 75%, more preferably at least 100% and even more preferably 200% more of the polypeptide encoded by said gene compared to the non-engineered cell in the same experimental or treatment conditions. For instance, one can mention the case of the cytidine deaminase (CDA) in T cell.

Said introduction of gene (or gene transfer) may be by transfection or other means, and the gene may be integrated in the genome or under a non-integrated form.

By “assaying the hypersensitivity to said drug, preferably prodrug, of the immune cell”, it is meant that an in vitro test is performed by contacting said engineered human cells, preferably human immune cells, with a series of different amounts of the prodrug and evaluating their survival rate (i.e determination of IC50 or slope of the dose—response curve). The concentration of such compound can be routinely and reliably measured by a given analytical method such as in WO201575195.

Hypersensitivity Towards (Pro)Drugs

Preferably the drug to which the engineered human cell is made hypersensitive is a prodrug. By the term “prodrug” is generally meant for a medication or compound that, after administration, is metabolized (i.e., converted within the body) into a pharmacologically active drug. Inactive prodrugs are pharmacologically inactive medications that are metabolized into an active form within the body.

Herein, the terms “prodrug” and “drug” can be used for the same compound respectively to mean that the compound is active (drug) or not yet active (prodrug) towards the engineered cell.

The prodrug encompassed in the scope of the present invention can be selected among the following list, but not limited to, Aceburic acid, Acemetacin, O-Acetylpsilocin, Aconiazide, Adrafinil, Alatrofloxacin, Aldophosphamide, Amfecloral, Amifostine, Amlodipine/benazepril, Amphetaminil, Ampiroxicam, 4-Androstenediol, 1-Androstenedione Arbaclofen placarbil, Aripiprazole lauroxil, Avizafone, Azathioprine, Bacampicillin, Bambuterol, Benazepril, Benzphetamine, Berefrine, Bezitramide, Bopindolol, Brincidofovir, Brivanib alaninate, Bupropion, 1,4-Butanediol, Capecitabine Carbamazepine Carfecillin Carindacillin Carisoprodol Cefuroxime axetil Chloral betaine Chloral hydrate 4-Chlorokynurenine, Chlorotrianisene, Cilazapril, Cinazepam, Clobenzorex, Clofibrate, Clofibride, Cloforex, Clopidogrel, Cloxazolam, Codeine Combretastatin A-4 phosphate CRL-40,941 Cyclophosphamide, Cyprodenate, Dasolampanel, Deflazacort, Delapril, D-Deprenyl, Dextromethorphan, DHA-clozapine, Dibutyrylmorphine, Dimethylamphetamine, Dipivefrine, Dirithromycin, Dolasetron, L-DOPA, Droxidopa, Enalapril, Estrobin, Etilevodopa, Etofibrate, Evofosfamide, Famciclovir, Fenofibrate, Fesoterodine, 5-formyl-2′-deoxycytidine (5fdC), Fosamprenavir, Fosaprepitant, Fosfluconazole, Fosinopril, Fosphenytoin, Fospropofol, Fostamatinib, Fostemsavir, Fursultiamine, Gabapentin enacarbil, Gidazepam, Glycerol, phenylbutyrate, Heroin, Hetacillin, Gamma-Hydroxybutyraldehyde, 5-hydroxymethyl-2′-deoxycytidine (5hmdC), Ibotenic acid, Imidapril, Indometacin, farnesil, Irinotecan, Isoniazid, Isophosphasmide, Leflunomide, Levomethorphan, Lisdexamfetamine, Loxoprofen, Melevodopa, Mesocarb, Mestranol, Methyl aminolevulinate, Midodrine, Milacemide, Moexipril, 6-Monoacetylmorphine, Nabumetone, Oxazolam, Parecoxib, Perindopril, Picamilon, Pirisudanol, Pivampicillin, Pivmecillinam, Potassium canrenoate Prednisone, Pretomanid, Proglumetacin, Proguanil, Prontosil, Protide, Pyrazinamide, Quinapril, R7, (drug), Rabeprazole, Ramipril, Rilmazafone, Romidepsin, Ronifibrate, Sacubitril, Sergliflozin etabonate, Sibrafiban, Sibutramine, Sodium phenylbutyrate Sofosbuvir, Spirapril, Spironolactone, Spiruchostatin, Sulindac, Tamoxifen, Taribavirin, Tebipenem, Tegafur, Temocapril, Temozolomide, Tenofovir, disoproxil, Terfenadine, Tolgabide, Trandolapril, Triclofos, Tybamate, Valaciclovir, Gamma-Valerolactone, Valganciclovir, Valofane, Varespladib, methyl, Ximelagatran and Zofenopril.

Are preferred the prodrugs which are used for being commonly used in the treatment of a wide range of cancers, including hematological malignancies (blood cancers, like leukemia and lymphoma), many types of carcinoma (solid tumors) and soft tissue sarcomas. Those prodrugs may be used in combination chemotherapy as a component of various chemotherapy regimens.

Further Engineering of Immune Cells to Make them Resistant to Another Specific Drug

Another aspect of the present invention relates to a method for further engineering human cell, preferably immune cell—already made hypersensitive by the above described method- to make it resistant to a specific drug, the latter being different to that used for hypersensitivity depletion.

This added attribute is particularly useful when immunotherapy using immune cells, especially CAR T cells is combined with chemotherapy in the treatment of cancerous indications; especially when specific drug, approved by National Drug Administrations, are being used.

This double genetic engineering to provide both hypersensitivity to one drug and resistance to another one may be very useful, especially for patients treated previously or concomitantly with chemotherapy or with a different lymphodepleting treatment. For instance, this method allows to making immune cells resistant to the drug used during the chemotherapy and/or immunosuppressive treatment, while keeping the possibility to deplete them by administration of another specific drug on demand.

Overexpression of CDA to Confer Hypersensitivity to Deoxycytidine Analogs

The immune cells according to the present invention, which CDA is expressed, are produced to be administered to the patient prior to their elimination by the deoxycytidine analogs drug in case of need (such as occurrence of an adverse event). Expression of CDA has been found by the inventors to confer sensitivity of cells derived from lymphoid progenitor cells, such as NK cells and T cells, to deoxycytidine analogs. Thus, to modulate or terminate the treatment, further administration of deoxycytidine analogs to which said cells have been made sensitive may be performed in order to deplete in vivo said cells.

According to one embodiment, the present invention relates to a method of producing human cell that may be depleted in-vivo as part of a cell therapy or immunotherapy treatment, said method comprising:

(a) Providing a human cell;

(b) Inducing hypersensitivity to deoxycytidine analogs into said cell by selectively expressing or overexpressing at least CDA transgene involved in the mechanism of action of said drug,

(c) Optionally assaying the hypersensitivity to said drug of said cell engineered in step b);

(d) Expanding said engineered cell obtained in step b).

According to one preferred embodiment, the gene encoding for the human CDA which is used in the present invention to be expressed is the one of SEQ ID NO. 20 (CAAACCATGGGAGGCTCCTCTCCTAGACCCTGCATCCTGAAAGCTGCGTACCTGAGAGCCTGCGGTCTGGCTG CAGGGACACACCCAAGGGGAGGAGCTGCAATCGTGTCTGGGGCCCCAGCCCAGGCTGGCCGGAGCTCCTGTT TCCCGCTGCTCTGCTGCCTGCCCGGGGTACCAACATGGCCCAGAAGCGTCCTGCCTGCACCCTGAAGCCTGAG TGTGTCCAGCAGCTGCTGGTTTGCTCCCAGGAGGCCAAGAAGTCAGCCTACTGCCCCTACAGTCACTTTCCTGT GGGGGCTGCCCTGCTCACCCAGGAGGGGAGAATCTTCAAAGGGTGCAACATAGAAAATGCCTGCTACCCGCT GGGCATCTGTGCTGAACGGACCGCTATCCAGAAGGCCGTCTCAGAAGGGTACAAGGATTTCAGGGCAATTGC TATCGCCAGTGACATGCAAGATGATTTTATCTCTCCATGTGGGGCCTGCAGGCAAGTCATGAGAGAGTTTGGC ACCAACTGGCCCGTGTACATGACCAAGCCGGATGGTACGTATATTGTCATGACGGTCCAGGAGCTGCTGCCCT CCTCCTTTGGGCCTGAGGACCTGCAGAAGACCCAGTGACAGCCAGAGAATGCCCACTGCCTGTAACAGCCACC TGGAGAACTTCATAAAGATGTCTCACAGCCCTGGGGACACCTGCCCAGTGGGCCCCAGCCCTACAGGGACTGG GCAAAGATGATGTTTCCAGATTACACTCCAGCCTGAGTCAGCACCCCTCCTAGCAACCTGCCTTGGGACTTAGA ACACCGCCGCCCCCTGCCCCACCTTTCCTTTCCTTCCTGTGGGCCCTCTTTCAAAGTCCAGCCTAGTCTGGACTG CTTCCCCATCAGCCTTCCCAAGGTTCTATCCTGTTCCGAGCAACTTTTCTAATTATAAACATCACAGAACATCCTG GATC) RefSeq n^{o} NM_001785.2, or a variant thereof comprising a nucleotide sequence that has at least 60%, such as at least 80%, at least 85%, at least 90% or at least 95%, sequence identity with the nucleotide sequence set forth in SEQ ID NO: 20 over the entire length of SEQ ID NO: 20. However, it is understood that due to the degeneration of the genetic code any other suitable nucleotide sequence coding for the amino acid sequence set forth in SEQ ID NO: 20 is also encompassed by the present disclosure.

Accordingly, in certain embodiment of the present invention, the human CDA to be expressed comprises a polypeptide of SEQ ID NO: 1 (MAQKRPACTLKPECVQQLLVCSQEAKKSAYCPYSHFPVGAALLTQEGRIFKGCNIENACYPLGICAERTAIQKAVSE GYKDFRAIAIASDMQDDFISPCGACRQVMREFGTNWPVYMTKPDGTYIVMTVQELLPSSFGPEDLQKTQ), corresponding to P32320 (CDD_HUMAN), or a variant thereof comprising an amino acid sequence that has at least 60%, such as at least 80%, at least 85%, at least 90% or at least 95%, sequence identity with the amino acid sequence set forth in SEQ ID NO: 1 over the entire length of SEQ ID NO: 1. The variant may comprise an amino acid sequence which has one or more, such as two, three, four, five or six amino acid substitutions compared to SEQ ID NO: 1. Preferably, such amino acid substitution is a conservative substitution which means that one amino acid is replaced by another one that is similar in size and chemical properties. Such conservative amino acid substitution may thus have minor effects on the peptide structure and can thus be tolerated without compromising function. Preferably, such variant is capable of maintaining the activity of CDA and is capable of catalyzing the deamination of cytidine and deoxycytidine to uridine and deoxyuridine.

According to a preferred embodiment, the immune cells according to the present invention, in which the CDA transgene is expressed, are administered to the patient prior to their elimination by a deoxycytidine analog drug. Thus, to modulate or terminate the treatment, further administration of a deoxycytidine analog to which said cells have been made sensitive may be performed in order to deplete in vivo said cells.

According to a preferred embodiment, said prodrug-hypersensitive engineered human cell, preferably immune cell being administered to the patient beforehand, which comprises administering to a patient the prodrug 5fdC and/or 5hmdC in case of need.

The compounds 5-hydroxymethyl-2′deoxycytidine (5hmdC) and 5-formy-2′deoxycytidine (5fdC) are oxidized forms of 5-methyl deoxycytosine (5mdC). The former -5-formyl deoxycytosine (5fdC) is highly mutagenic, capable of driving both C-to-T transitions and C-to-A transversions (Karino, N. et al., 2001, Nucleic Acids Res. 29:2456-2463). The second one -5-Hydroxymethylcytosine- has been found strongly depleted in human cancers (Jin S G et al, 2011, Cancer Res.; 71(24):7360-5).

The cytidine analog 5-hydroxymethyl-2′ deoxycytidine (called herein 5hmdC), is an epigenetically modified form of cytosine that is normally metabolized by cytidine deaminase (CDA) and transformed into its corresponding Uridine counterpart (5hmdU). Once generated, 5hmdU is phosphorylated and eventually incorporated into DNA by DNA polymerase. Incorporated 5hmdU is recognized as damaged bases and trigger extensive uracil glycosylase activity that results in DNA breaks and cytotoxicity. CDA compete with deoxycytidine kinase (called herein) dCK for 5hmdC metabolization. In certain type of cancer cells however, CDA expression shift this steady state equilibrium by outcompeting dCK activity. This results in the transformation of 5hmdC into 5hmdU that lead to the aforementioned cytotoxicity.

The doses of each drug or prodrug administrated for in vivo depleting engineered prodrug-hypersensitive human cell, preferably immune cell may correspond essentially to the ones used in the clinical trials (clinicaltrial.com) and agreed by national health authorities.

A dose ranging between 50 and 1000 mg of 5fdC or 5hmdC, advantageously between 100 mg and 500 mg of 5fdC and/or 5hmdC may be administrated to the patient per day. Preferably the administration of said drug(s) is performed by intravenous infusion. Administrations may be repeated for during a month-cycle.

Further Engineering of CDA-Overexpressing Engineered Human Cells

In a particular embodiment, said engineered cells of the present invention can advantageously combine an expression of CDA gene to confer hypersensitivity to deoxycytidine analogs and said and a further genetic engineering to confer specific resistance to another drug, such additional modification may be performed by a gene inactivation or by overexpression of a wild-type or a mutant form of a gene, said gene being involved in the metabolization of said second drug.

In another particular embodiment, said further genetic engineered of cells according to the present invention, in addition to the gene expression of CDA, confers resistance to said second drug selected in the group consisting of alkylating agents (other than cyclophosphamide and isophosphamide), metabolic antagonists (e.g., purine nucleoside antimetabolite such as clofarabine, fludarabine or 2′-deoxyadenosine, 5-fluorouracil or derivatives thereof), antitumor antibiotics (e.g., mitomycin, adriamycin), plant-derived antitumor agents (e.g., vincristine, vindesine, Taxol), cisplatin, carboplatin, etoposide, TRIMETHOTRIXATE™ (TMTX), TEMOZOLOMIDE™, RALTRITREXED™, S-(4-Nitrobenzyl)-6-thioinosine (NBMPR),6-benzyguanidine (6-BG), bis-chloronitrosourea (BCNU) and CAMPTOTHECIN™, immunomodulating agents such as thalidomide (Thalomid®) Lenalidomide (Revlimid®) Pomalidomide (Pomalyst®), proteasome inhibitors such as Bortezomib (Velcade®), Carfilzomib (Kyprolis®), Histone deacetylase (HDAC) inhibitors such as Panobinostat (Farydak®), or a therapeutic derivative of any thereof.

In a more particular embodiment, said engineered cells of the present invention can advantageously combine an expression of CDA gene to confer hypersensitivity to deoxycytidine analogs and said further genetic engineering being a gene inactivation of a gene selected in the group of deoxycytidine kinase (dCk), hypoxanthine guanine phosphoribosyl transferase (HPRT), glucocorticoid receptor (GR) and CD52, conferring specific drug resistance to purine nucleoside analogues (PNAs)—such as clofarabine or fludarabine—, corticosteroids, alemtuzumab respectively.

Referring to the previous embodiment, said engineered cells of the present invention can advantageously combine an expression of CDA gene to confer hypersensitivity to deoxycytidine analogs (such as 5hmdC or 5fdC) and said further genetic engineering is inactivation of dCk gene conferring specific drug resistance to purine nucleoside analogues (PNAs)—such as clofarabine, fludarabine or cladribine.

Here is an approach to render normal cells sensitive to 5hmdC or 5fdC to mimic what happens in cancer cells by combining both the expression of CDA and the inactivation of dCK as in normal cells CDA is not sufficiently expressed to efficiently metabolized 5hmdC or 5fdC into 5hmdU. This can redirect 5hmdC metabolization flux through CDA activity, eventually leading to 5hmdU production and thus cellular toxicity.

In one embodiment, drug sensitizing gene which can be inactivated to confer drug resistance to the T-cell is the human deoxycytidine kinase (dCK) gene. Deoxycytidine kinase (dCK)—human Uniprot ref: P27707) is required for the phosphorylation of the deoxyribonucleosides deoxycytidine (dC), deoxyguanosine (dG) and deoxyadenosine (dA). This enzyme is required for the phosphorylation of the deoxyribonucleosides deoxycytidine (dC), deoxyguanosine (dG) and deoxyadenosine (dA). Purine nucleotide analogs (PNAs) are metabolized by dCK into mono-, di- and tri-phosphate PNA. Their triphosphate forms and particularly clofarabine triphosphate compete with ATP for DNA synthesis, acts as proapoptotic agent and are potent inhibitors of ribonucleotide reductase (RNR) which is involved in trinucleotide production. It is also an essential enzyme for the phosphorylation of numerous nucleoside analogs widely employed as antiviral and chemotherapeutic agents. Deficiency of DCK is associated with resistance to antiviral and anticancer chemotherapeutic agents. DCK is frequently inactivated in acquired gemcitabine-resistant human cancer cells (Saiki Y et al, 2012, Biochem Biophys Res Commun. 21(1):98-104). Inactivation of dCK increased primary T cells resistance to clofarabine (Valton J et al, 2014, Molecular Therapy; 23 (9), 1507-15183).

According to a preferred embodiment, said human dCK inhibition is performed by a least one rare-cutting endonuclease which gene target has RefSeq NM_000788. Said endonuclease preferably targets SEQ ID NO:17, or to a sequence having at least 95% identity with the SEQ ID NO:17.

According to a preferred embodiment, the inactivation of the target gene of SEQ ID NO. 17 encoding for human dCK enzyme in T cells is mediated by TALE nuclease.

According to a more preferred embodiment, said human dCK enzyme inhibition is performed by a least one rare-cutting endonuclease which targets a sequence of SEQ ID NO:17, or to a sequence having at least 95% identity with the SEQ ID NO:17.

Preferably, the inactivation of dCK in T cells is mediated by TALE nuclease. To achieve this goal, several pairs of dCK TALE-nuclease have been designed, assembled at the polynucleotide level and validated by sequencing. Examples of TALE-nuclease pairs which can be used according to the invention are depicted by SEQ ID N^{o} 18 and SEQ ID N^{o} 19. In addition, this dCK inactivation in T cells confers resistance to purine nucleoside analogs (PNAs) such as clofarabine and fludarabine.

According to a more preferred embodiment, the method of the present invention comprises a step of gene overexpression in immune cells of CDA which encodes for cytidine deaminase to confer hypersensitivity to the drug such as 5hmdC or 5FdC, and a step of inactivation in said immune cells of dCK which confers resistance to drug such as clofarabine or fludarabine.

As exemplary embodiments, said engineered cells of the present invention can advantageously combine an expression of CDA gene to confer hypersensitivity to deoxycytidine analogs and said further genetic engineering being a gene expression (co-expression) of a mutated gene selected in the group consisting of dihydrofolate reductase (DHFR) inosine monophosphate dehydrogenase 2 (IMPDH2), calcineurin (PP2B) and methylguanine transferase (MGMT), conferring specific drug resistance to respectively anti-folate preferably methotrexate (MTX), to MPDH inhibitors such as mycophenolic acid (MPA) or its prodrug mycophenolate mofetil (MMF), to calcineurin inhibitor such as FK506 and/or Cs and to alkylating agents, such as nitrosoureas and temozolomide (TMZ).

The above mutated genes such as DHFR, IMPDH2, PP2B, MGMT can be obtained such as described in WO 2015075195.

Another example of enzyme which can be inactivated is human hypoxanthine-guanine phosphoribosyl transferase (HPRT) gene (Genbank: M26434.1). In particular HPRT can be inactivated in engineered human cell, preferably T-cells to confer resistance to a cytostatic metabolite, the 6-thioguanine (6TG) which is converted by HPRT to cytotoxic thioguanine nucleotide and which is currently used to treat patients with cancer, in particular leukemia (Hacke, Treger et al. 2013). Guanines analogs are metabolized by HPRT transferase that catalyzes addition of phosphoribosyl moiety and enables the formation of TGMP. Guanine analogues including 6 mercapthopurine (6MP) and 6 thioguanine (6TG) are usually used as lymphodepleting drugs to treat ALL. They are metabolized by HPRT (hypoxanthine phosphoribosyl transferase that catalyzes addition of phosphoribosyl moiety and enables formation TGMP. Their subsequent phosphorylations lead to the formation of their triphosphorylated forms that are eventually integrated into DNA. Once incorporated into DNA, thio GTP impairs fidelity of DNA replication via its thiolate groupment and generate random point mutation that are highly deleterious for cell integrity.

In another embodiment, the inactivation of the CD3 normally expressed at the surface of the T-cell can confer resistance to anti-CD3 antibodies such as teplizumab.

In another particular embodiment, the inventors sought to develop an “off-the shelf” immunotherapy strategy, using allogeneic T-cells resistant to multiple drugs to mediate selection of engineered human cell, preferably T-cells when the patient is treated with different drugs. The therapeutic efficiency can be significantly enhanced by genetically engineering multiple drug resistance allogeneic T-cells. Such a strategy can be particularly effective in treating tumors that respond to drug combinations that exhibit synergistic effects. Moreover multiple resistant engineered human cell, preferably T-cells can expand and be selected using minimal dose of drug agents.

Thus, the method according to the present invention can comprise modifying T-cell to confer multiple drug resistance to said T-cell. Said multiple drug resistance can be conferred by either expressing more than one drug resistance gene or by inactivating more than one drug sensitizing gene. In another particular embodiment, the multiple drug resistance can be conferred to said T-cell by expressing at least one drug resistance gene and inactivating at least one drug sensitizing gene. In particular, the multiple drug resistance can be conferred to said T-cell by expressing at least one drug resistance gene such as mutant form of DHFR, mutant form of IMPDH2, mutant form of calcineurin, mutant form of MGMT, the ble gene, and the mcrA gene and inactivating at least one drug sensitizing gene such as HPRT gene. In a preferred embodiment, multiple drug resistance can be conferred by inactivating HPRT gene and expressing a mutant form of DHFR; or by inactivating HPRT gene and expressing a mutant form of IMPDH2; or by inactivating HPRT gene and expressing a mutant form of calcineurin; by inactivating HPRT gene and expressing a mutant form of MGMT; by inactivating HPRT gene and expressing the ble gene; by inactivating HPRT gene and expressing the mcrA gene.

As other exemplary embodiments, said engineered cells of the present invention can advantageously combine an expression of CDA gene to confer hypersensitivity to deoxycytidine analogs and said further genetic engineering being a gene expression of a wild type gene selected in the group consisting of MDR1, ble and mcrA, conferring specific drug resistance to respectively MDR1 resistance drugs such as 4-nitroquinoline-N-oxide, cerulenin, and brefeldin A, to bleomycin and to mitomycin C.

According to another embodiment, said engineered cells of the present invention can advantageously combine an expression of CDA gene to confer hypersensitivity to deoxycytidine analogs and said further genetic engineering is an expression of another gene which confers a supplemental hypersensitivity to another specific drug, preferably one gene selected in the group consisting of CYP2D6-2, CYP2C9, CYP3A4, CYP2D6-1, CYP2C19 and CYP1A2 conferring drug-specific hypersensitivity to cyclophosphamide and/or isophosphamide.

It also encompassed in the scope of the present invention the possibility to make human cell, preferably human immune cell, hypersensitive to at least two different drugs, ie. said cell is endowed with at least two specific drug hypersensitivity. This embodiment is particularly useful to remedy to the case when a number of cells escape from the effect of the first hypersensitivity by implementing an additional hypersensitivity mechanism.

Said method can be used to produce engineered cells for treating cancer, infection or immune disease in a patient by unique or sequential administration thereof to a patient.

As a preferred embodiment, the invention provides the administration of an immune cell made hypersensitive to deoxycytidine analogs drug by expressing the CDA gene, said cell being further engineered to endow a chimeric antigen receptor (CAR) against said cancerous cell, infectious agent or dysfunctioning host immune cell.

Overexpression of Particular Cytochrome Gene(s) to Confer Hypersensitivity to Cyclophosphamide and/or Isophosphamide

According to one preferred embodiment, the gene to be overexpressed in step (b) of the present method of the invention is selected in the group consisting of the P450 cytochromes family, and said human cell, preferably immune cell is hypersensitive to cyclophosphamide and/or isophosphamide.

Several P450 cytochromes are expressed in hepatocyte by few of them bear specificity towards cyclosphosphamide and isophosphamide. According to Chang T K et al (1997) and Chang T K et al; (1993), respectively CYP2C19, and CYP3A and CYP2B6 were reported to be proficient to do so. Because these enzymes are not expressed in T cells, cyclophosphamide and isophosphamide need to be first metabolized in hepatocytes then transported in the blood in their activated forms before being internalized in T cells. Once in the T cells, their alkylating properties promote DNA and macromolecule damages that engender cell death. The dose of cyclophosphamide used in clinic to promote T cell depletion is usually set a daily dose of 500 mg/m2 (ie. Book “Oxford Desk Reference: Oncology” by T V Ajithkumar, A Barrett, H Hatcher and N Cook, 2011, Oxford University Press), a dose at which secondary adverse events are not anymore negligible.

According to a more preferred embodiment, said gene to be overexpressed in step (b) of the present method of the invention is selected in the group consisting of CYP2D6-2, CYP2C9, CYP3A4, CYP2D6-1, CYP2C19 and CYP1A2, and said engineered human cell, preferably T cell, is hypersensitive to cyclophosphamide and/or isophosphamide. Among all the P450 cytochromes identified so far in human (more than 60 CYP according to https://ghr.nlm.nih.gov/geneFamily/cyp) which are mainly expressed in liver and not or very little in immune cells, it appears that few of them bear a specificity towards a particular drug. This is the case for some of them, the ones listed above—CYP2D6-2, CYP2C9, CYP3A4, CYP2D6-1, CYP2C19 and CYP1A2—which are specific to the prodrug isophosphamide and/or cyclophosphamide.

Overexpression of CYP2D6-2 to Confer Hypersensitivity to Cyclosphosphamide and/or Isophosphamide

The immune cells according to the present invention, which CYP2D6-2 is expressed, are produced to be administered to the patient prior to their elimination by the cyclosphosphamide and/or isophosphamide drug in case of need (such as occurrence of an adverse event). Expression of CYP2D6-2 has been found by the inventors to confer sensitivity of cells derived from lymphoid progenitor cells, such as NK cells and T cells, to cyclosphosphamide and/or isophosphamide. Thus, to modulate or terminate the treatment, further administration of cyclosphosphamide and/or isophosphamide to which said cells have been made sensitive may be performed in order to deplete in vivo said cells.

According to one embodiment, the present invention relates to a method of producing human cell that may be depleted in-vivo as part of a cell therapy or immunotherapy treatment, said method comprising:

(a) Providing a human cell;

(b) Inducing hypersensitivity to cyclosphosphamide and/or isophosphamide into said cell by selectively expressing or overexpressing at least CYP2D6-2 transgene involved in the mechanism of action of said drug,

(c) Optionally assaying the hypersensitivity to said drug of said cell engineered in step b);

(d) Expanding said engineered cell obtained in step b).

In another embodiment, the gene encoding for the human cytochrome P450 2D6 isoform2 (CYP2D6_2) which is used in the present invention to be expressed is the one of SEQ ID NO. 24 (GTGCTGAGAGTGTCCTGCCTGGTCCTCTGTGCCTGGTGGGGTGGGGGTGCCAGGTGTGTCCAGAGGAGCCC ATTTGGTAGTGAGGCAGGTATGGGGCTAGAAGCACTGGTGCCCCTGGCCGTGATAGTGGCCATCTTCCTGCTC CTGGTGGACCTGATGCACCGGCGCCAACGCTGGGCTGCACGCTACCCACCAGGCCCCCTGCCACTGCCCGGGC TGGGCAACCTGCTGCATGTGGACTTCCAGAACACACCATACTGCTTCGACCAGTTGCGGCGCCGCTTCGGGGA CGTGTTCAGCCTGCAGCTGGCCTGGACGCCGGTGGTCGTGCTCAATGGGCTGGCGGCCGTGCGCGAGGCGCT GGTGACCCACGGCGAGGACACCGCCGACCGCCCGCCTGTGCCCATCACCCAGATCCTGGGTTTCGGGCCGCGT TCCCAAGGGGTGTTCCTGGCGCGCTATGGGCCCGCGTGGCGCGAGCAGAGGCGCTTCTCCGTGTCCACCTTGC GCAACTTGGGCCTGGGCAAGAAGTCGCTGGAGCAGTGGGTGACCGAGGAGGCCGCCTGCCTTTGTGCCGCCT TCGCCAACCACTCCGGACGCCCCTTTCGCCCCAACGGTCTCTTGGACAAAGCCGTGAGCAACGTGATCGCCTCC CTCACCTGCGGGCGCCGCTTCGAGTACGACGACCCTCGCTTCCTCAGGCTGCTGGACCTAGCTCAGGAGGGAC TGAAGGAGGAGTCGGGCTTTCTGCGCGAGGTGCTGAATGCTGTCCCCGTCCTCCTGCATATCCCAGCGCTGGC TGGCAAGGTCCTACGCTTCCAAAAGGCTTTCCTGACCCAGCTGGATGAGCTGCTAACTGAGCACAGGATGACC TGGGACCCAGCCCAGCCCCCCCGAGACCTGACTGAGGCCTTCCTGGCAGAGATGGAGAAGGCCAAGGGGAAC CCTGAGAGCAGCTTCAATGATGAGAACCTGCGCATAGTGGTGGCTGACCTGTTCTCTGCCGGGATGGTGACCA CCTCGACCACGCTGGCCTGGGGCCTCCTGCTCATGATCCTACATCCGGATGTGCAGCGCCGTGTCCAACAGGA GATCGACGACGTGATAGGGCAGGTGCGGCGACCAGAGATGGGTGACCAGGCTCACATGCCCTACACCACTGC CGTGATTCATGAGGTGCAGCGCTTTGGGGACATCGTCCCCCTGGGTGTGACCCATATGACATCCCGTGACATC GAAGTACAGGGCTTCCGCATCCCTAAGGGAACGACACTCATCACCAACCTGTCATCGGTGCTGAAGGATGAGG CCGTCTGGGAGAAGCCCTTCCGCTTCCACCCCGAACACTTCCTGGATGCCCAGGGCCACTTTGTGAAGCCGGA GGCCTTCCTGCCTTTCTCAGCAGGCCGCCGTGCATGCCTCGGGGAGCCCCTGGCCCGCATGGAGCTCTTCCTCT TCTTCACCTCCCTGCTGCAGCACTTCAGCTTCTCGGTGCCCACTGGACAGCCCCGGCCCAGCCACCATGGTGTCT TTGCTTTCCTGGTGAGCCCATCCCCCTATGAGCTTTGTGCTGTGCCCCGCTAGAATGGGGTACCTAGTCCCCAG CCTGCTCCCTAGCCAGAGGCTCTAATGTACAATAAAGCAATGTGGTAGTTCCA)RefSeq n^{o} NP_000097, or a variant thereof comprising an nucleotide sequence that has at least 60%, such as at least 80%, at least 85%, at least 90% or at least 95%, sequence identity with the nucleotide sequence set forth in SEQ ID NO: 24 over the entire length of SEQ ID NO: 24. However, it is understood that due to the degeneration of the genetic code any other suitable nucleotide sequence coding for the amino acid sequence set forth in SEQ ID NO: 24 is also encompassed by the present disclosure.

Accordingly, in certain embodiment of the present invention, the human cytochrome P450 2D6 isoform 2 to be expressed comprises a polypeptide of SEQ ID NO: 2 (MGLEALVPLAVIVAIFLLLVDLMHRRQRWAARYPPGPLPLPGLGNLLHVDFQNTPYCFDQLRRRFGDVFSLQLAW TPVVVLNGLAAVREALVTHGEDTADRPPVPITQILGFGPRSQGRPFRPNGLLDKAVSNVIASLTCGRRFEYDDPRFLR LLDLAQEGLKEESGFLREVLNAVPVLLHIPALAGKVLRFQKAFLTQLDELLTEHRMTWDPAQPPRDLTEAFLAEMEK AKGNPESSFNDENLCIVVADLFSAGMVTTSTTLAWGLLLMILHPDVQRRVQQEIDDVIGQVRRPEMGDQAHMPY TTAVIHEVQRFGDIVPLGVTHMTSRDIEVQGFRIPKGTTLITNLSSVLKDEAVWEKPFRFHPEHFLDAQGHFVKPEAF LPFSAGRRACLGEPLARMELFLFFTSLLQHFSFSVPTGQPRPSHHGVFAFLVTPSPYELCAVPR), corresponding to P10635 (Ref Uniprot), or a variant thereof comprising an amino acid sequence that has at least 60%, such as at least 80%, at least 85%, at least 90% or at least 95%, sequence identity with the amino acid sequence set forth in SEQ ID NO: 2 over the entire length of SEQ ID NO: 2. The variant may comprise an amino acid sequence which has one or more, such as two, three, four, five or six amino acid substitutions compared to SEQ ID NO: 2. Preferably, such amino acid substitution is a conservative substitution which means that one amino acid is replaced by another one that is similar in size and chemical properties. Such conservative amino acid substitution may thus have minor effects on the peptide structure and can thus be tolerated without compromising function. Preferably, such variant is capable of maintaining the activity of human cytochrome P450 2D6 isoform 2 and metabolizing and eliminating clinically used drugs, in a process referred to as O-demethylation.

According to a preferred embodiment, the immune cells according to the present invention, in which the CYP2D6-2 transgene is expressed, are administered to the patient prior to their elimination by isophosphamide and/or cyclophosphamide drug. Thus, to modulate or terminate the treatment, further administration of isophosphamide and/or cyclophosphamide to which said cells have been made sensitive may be performed in order to deplete in vivo said cells.

According to a preferred embodiment, said prodrug-hypersensitive engineered human cell, preferably immune cell being administered to the patient beforehand, which comprises administering to a patient isophosphamide or cyclophosphamide in case of need.

These 2 alkylating agents have the advantage of being used to deplete engineered immune cells, preferably CAR T cells, in case of occurrence of a serious adverse event, but also, as chemical drug approved by National Drug Administration, can be used for treating cancerous diseases such as lymphomas, some forms of brain cancer, leukemia, and some solid tumors (Takimoto C H, Calvo E. “Principles of Oncologic Pharmacotherapy” in Pazdur R, Wagman L D, Camphausen; Young S D et al, 2006, Clinical Cancer Research 12 (10): 3092-8).

According to another preferred embodiment, said prodrug-hypersensitive engineered human cell, preferably immune cell being administered to the patient beforehand, which comprises administering to a patient the prodrug isophosphamide and/or cyclophosphamide in case i.e. of occurrence of an adverse event.

A dose ranging between 100 mg and 12,000 mg of isophosphamide, advantageously between 1000 mg and 8 000 mg of isophosphamide may be administrated to the patient per day.

A dose ranging between 1,000 mg and 7,000 mg of cyclophosphamide, advantageously between 2,000 mg and 5,000 mg of cyclophosphamide may be administrated to the patient per day.

The above embodiments relative to the dose of these drugs are relevant for all cases described herein when human cells are engineered by expressing or overexpressing at least one gene selected in the group consisting of CYP2D6-2, CYP2C9, CYP3A4, CYP2D6-1, CYP2C19 and CYP1A2.

In a particular embodiment, said engineered cells of the present invention can advantageously combine an expression of CYP2D6-2 gene to confer hypersensitivity to cyclosphosphamide and/or isophosphamide and said and a further genetic engineering to confer specific resistance to another drug, such additional modification may be performed by a gene inactivation or by overexpression of a wild-type or a mutant form of a gene, said gene being involved in the metabolization of said second drug.

In another particular embodiment, said further genetic engineering of cells according to the present invention, in addition to the gene expression of CYP2D6-2, confers resistance to said second drug selected in the group consisting of alkylating agents (other than cyclophosphamide and isophosphamide), metabolic antagonists (e.g., purine nucleoside antimetabolite such as clofarabine, fludarabine or 2′-deoxyadenosine, 5-fluorouracil or derivatives thereof), antitumor antibiotics (e.g., mitomycin, adriamycin), plant-derived antitumor agents (e.g., vincristine, vindesine, Taxol), cisplatin, carboplatin, etoposide, TRIMETHOTRIXATE™ (TMTX), TEMOZOLOMIDE™, RALTRITREXED™, S-(4-Nitrobenzyl)-6-thioinosine (NBMPR),6-benzyguanidine (6-BG), bis-chloronitrosourea (BCNU) and CAMPTOTHECIN™, immunomodulating agents such as thalidomide (Thalomid®) Lenalidomide (Revlimid®) Pomalidomide (Pomalyst®), proteasome inhibitors such as Bortezomib (Velcade®), Carfilzomib (Kyprolis®), Histone deacetylase (HDAC) inhibitors such as Panobinostat (Farydak®), or a therapeutic derivative of any thereof.

In a more particular embodiment, said engineered cells of the present invention can advantageously combine an expression of CYP2D6-2 gene to confer hypersensitivity to cyclosphosphamide and/or isophosphamide and said further genetic engineering being a gene inactivation of a gene selected in the group of deoxycytidine kinase (dCk), hypoxanthine guanine phosphoribosyl transferase (HPRT), glucocorticoid receptor (GR) and CD52, conferring specific drug resistance to purine nucleoside analogues (PNAs)—such as clofarabine or fludarabine—, corticosteroids, alemtuzumab respectively.

Referring to the previous embodiment, said engineered cells of the present invention can advantageously combine an expression of CYP2D6-2 gene to confer hypersensitivity to cyclosphosphamide and/or isophosphamide and said further genetic engineering is inactivation of dCk gene conferring specific drug resistance to purine nucleoside analogues (PNAs)—such as clofarabine or fludarabine.

As exemplary embodiments, said engineered cells of the present invention can advantageously combine an expression of CYP2D6-2 gene to confer hypersensitivity to cyclosphosphamide and/or isophosphamide and said further genetic engineering being a gene expression of a mutated gene selected in the group consisting of dihydrofolate reductase (DHFR) inosine monophosphate dehydrogenase 2 (IMPDH2), calcineurin (PP2B) and methylguanine transferase (MGMT), conferring specific drug resistance to respectively anti-folate preferably methotrexate (MTX), to MPDH inhibitors such as mycophenolic acid (MPA) or its prodrug mycophenolate mofetil (MMF), to calcineurin inhibitor such as FK506 and/or Cs and to alkylating agents, such as nitrosoureas and temozolomide (TMZ).

The above mutated genes such as DHFR, IMPDH2, PP2B, MGMT can be obtained such as described in WO 2015075195.

As other exemplary embodiments, said engineered cells of the present invention can advantageously combine an expression of CYP2D6-2 gene to confer hypersensitivity to cyclosphosphamide and/or isophosphamide and said further genetic engineering being a gene expression of a wild type gene selected in the group consisting of MDR1, ble and mcrA, conferring specific drug resistance to respectively MDR1 resistance drugs such as 4-nitroquinoline-N-oxide, cerulenin, and brefeldin A, to bleomycin and to mitomycin C.

According to another embodiment, said engineered cells of the present invention can advantageously combine an expression of CYP2D6-2 gene to confer hypersensitivity to cyclosphosphamide and/or isophosphamide and said further genetic engineering is an expression of another gene which confers a supplemental hypersensitivity to another specific drug, preferably one gene selected in the group consisting of CYP2C9, CYP3A4, CYP2D6-1, CYP2C19 and CYP1A2 conferring drug-specific hypersensitivity to cyclophosphamide and/or isophosphamide.

Said method can be used to produce engineered cells for treating cancer, infection or immune disease in a patient by unique or sequential administration thereof to a patient.

As a preferred embodiment, the invention provides the administration of an immune cell made hypersensitive to cyclosphosphamide and/or isophosphamide drug by expressing the CYP2D6-2 gene, said cell being further engineered to endow a chimeric antigen receptor (CAR) against said cancerous cell, infectious agent or dysfunctioning host immune cell.

Overexpression of CYP2C9 to Confer Hypersensitivity to Cyclosphosphamide and/or Isophosphamide

The immune cells according to the present invention, which CYP2C9 is expressed, are produced to be administered to the patient prior to their elimination by the cyclosphosphamide and/or isophosphamide drug in case of need (such as occurrence of an adverse event). Expression of CYP2C9 has been found by the inventors to confer sensitivity of cells derived from lymphoid progenitor cells, such as NK cells and T cells, to cyclosphosphamide and/or isophosphamide. Thus, to modulate or terminate the treatment, further administration of cyclosphosphamide and/or isophosphamide to which said cells have been made sensitive may be performed in order to deplete in vivo said cells.

According to one embodiment, the present invention relates to a method of producing human cell that may be depleted in-vivo as part of a cell therapy or immunotherapy treatment, said method comprising:

(a) Providing a human cell;

(b) Inducing hypersensitivity to cyclosphosphamide and/or isophosphamide into said cell by selectively expressing or overexpressing at least CYP2C9 transgene involved in the mechanism of action of said drug,

(c) Optionally assaying the hypersensitivity to said drug of said cell engineered in step b);

(d) Expanding said engineered cell obtained in step b).

In another specific embodiment, the gene encoding for the human cytochrome P450 2C9 precursor which is used in the present invention to be expressed is the one of SEQ ID NO. 22 (GTCTTAACAAGAAGAGAAGGCTTCAATGGATTCTCTTGTGGTCCTTGTGCTCTGTCTCTCATGTTTGCTTCTCCT TTCACTCTGGAGACAGAGCTCTGGGAGAGGAAAACTCCCTCCTGGCCCCACTCCTCTCCCAGTGATTGGAAATA TCCTACAGATAGGTATTAAGGACATCAGCAAATCCTTAACCAATCTCTCAAAGGTCTATGGCCCTGTGTTCACTC TGTATTTTGGCCTGAAACCCATAGTGGTGCTGCATGGATATGAAGCAGTGAAGGAAGCCCTGATTGATCTTGG AGAGGAGTTTTCTGGAAGAGGCATTTTCCCACTGGCTGAAAGAGCTAACAGAGGATTTGGAATTGTTTTCAGC AATGGAAAGAAATGGAAGGAGATCCGGCGTTTCTCCCTCATGACGCTGCGGAATTTTGGGATGGGGAAGAGG AGCATTGAGGACCGTGTTCAAGAGGAAGCCCGCTGCCTTGTGGAGGAGTTGAGAAAAACCAAGGCCTCACCC TGTGATCCCACTTTCATCCTGGGCTGTGCTCCCTGCAATGTGATCTGCTCCATTATTTTCCATAAACGTTTTGATT ATAAAGATCAGCAATTTCTTAACTTAATGGAAAAGTTGAATGAAAACATCAAGATTTTGAGCAGCCCCTGGATC CAGATCTGCAATAATTTTTCTCCTATCATTGATTACTTCCCGGGAACTCACAACAAATTACTTAAAAACGTTGCTT TTATGAAAAGTTATATTTTGGAAAAAGTAAAAGAACACCAAGAATCAATGGACATGAACAACCCTCAGGACTT TATTGATTGCTTCCTGATGAAAATGGAGAAGGAAAAGCACAACCAACCATCTGAATTTACTATTGAAAGCTTG GAAAACACTGCAGTTGACTTGTTTGGAGCTGGGACAGAGACGACAAGCACAACCCTGAGATATGCTCTCCTTC TCCTGCTGAAGCACCCAGAGGTCACAGCTAAAGTCCAGGAAGAGATTGAACGTGTGATTGGCAGAAACCGGA GCCCCTGCATGCAAGACAGGAGCCACATGCCCTACACAGATGCTGTGGTGCACGAGGTCCAGAGATACATTG ACCTTCTCCCCACCAGCCTGCCCCATGCAGTGACCTGTGACATTAAATTCAGAAACTATCTCATTCCCAAGGGCA CAACCATATTAATTTCCCTGACTTCTGTGCTACATGACAACAAAGAATTTCCCAACCCAGAGATGTTTGACCCTC ATCACTTTCTGGATGAAGGTGGCAATTTTAAGAAAAGTAAATACTTCATGCCTTTCTCAGCAGGAAAACGGATT TGTGTGGGAGAAGCCCTGGCCGGCATGGAGCTGTTTTTATTCCTGACCTCCATTTTACAGAACTTTAACCTGAA ATCTCTGGTTGACCCAAAGAACCTTGACACCACTCCAGTTGTCAATGGATTTGCCTCTGTGCCGCCCTTCTACCA GCTGTGCTTCATTCCTGTCTGAAGAAGAGCAGATGGCCTGGCTGCTGCTGTGCAGTCCCTGCAGCTCTCTTTCC TCTGGGGCATTATCCATCTTTCACTATCTGTAATGCCTTTTCTCACCTGTCATCTCACATTTTCCCTTCCCTGAAGA TCTAGTGAACATTCGACCTCCATTACGGAGAGTTTCCTATGTTTCACTGTGCAAATATATCTGCTATTCTCCATAC TCTGTAACAGTTGCATTGACTGTCACATAATGCTCATACTTATCTAATGTTGAGTTATTAATATGTTATTATTAAA TAGAGAAATATGATTTGTGTATTATAATTCAAAGGCATTTCTTTTCTGCATGTTCTAAATAAAAAGCATTATTAT TTGCTGA) REFSEQ: accession NP_000762) or a variant thereof comprising an nucleotide sequence that has at least 60%, such as at least 80%, at least 85%, at least 90% or at least 95%, sequence identity with the amino acid sequence set forth in SEQ ID NO: 22 over the entire length of SEQ ID NO: 22.

Accordingly, said human cytochrome P450 2C9 to be expressed comprises a polypeptide of SEQ ID NO 3 (MDSLVVLVLCLSCLLLLSLWRQSSGRGKLPPGPTPLPVIGNILQIGIKDISKSLTNLSKVYGPVFTLYFGLKPIVVLHGYE AVKEALIDLGEEFSGRGIFPLAERANRGFGIVFSNGKKWKEIRRFSLMTLRNFGMGKRSIEDRVQEEARCLVEELRKT KASPCDPTFILGCAPCNVICSIIFHKRFDYKDQQFLNLMEKLNENIKILSSPWIQICNNFSPIIDYFPGTHNKLLKNVAF MKSYILEKVKEHQESMDMNNPQDFIDCFLMKMEKEKHNQPSEFTIESLENTAVDLFGAGTETTSTTLRYALLLLLKH PEVTAKVQEEIERVIGRNRSPCMQDRSHMPYTDAVVHEVQRYIDLLPTSLPHAVTCDIKFRNYLIPKGTTILISLTSVL HDNKEFPNPEMFDPHHFLDEGGNFKKSKYFMPFSAGKRICVGEALAGMELFLFLTSILQNFNLKSLVDPKNLDTTPV VNGFASVPPFYQLCFIPV (Uniprot ref: P11712), or a variant thereof comprising an nucleotide sequence that has at least 60%, such as at least 80%, at least 85%, at least 90% or at least 95%, sequence identity with the nucleotide sequence set forth in SEQ ID NO: 3 over the entire length of SEQ ID NO: 3. The variant may comprise an amino acid sequence which has one or more, such as two, three, four, five or six amino acid substitutions compared to SEQ ID NO: 3. Preferably, such amino acid substitution is a conservative substitution which means that one amino acid is replaced by another one that is similar in size and chemical properties. Such conservative amino acid substitution may thus have minor effects on the peptide structure and can thus be tolerated without compromising function. Preferably, such variant is capable of maintaining the activity of cytochrome P450 2C9 precursor and is capable of oxidizing both xenobiotic and endogenous compounds.

In a particular embodiment, said engineered cells of the present invention can advantageously combine an expression of CYP2C9 gene to confer hypersensitivity to cyclosphosphamide and/or isophosphamide and said and a further genetic engineering to confer specific resistance to another drug, such additional modification may be performed by a gene inactivation or by overexpression of a wild-type or a mutant form of a gene, said gene being involved in the metabolization of said second drug.

In another particular embodiment, said further genetic engineered of cells according to the present invention, in addition to the gene expression of CYP2C9, confers resistance to said second drug selected in the group consisting of alkylating agents (other than cyclophosphamide and isophosphamide), metabolic antagonists (e.g., purine nucleoside antimetabolite such as clofarabine, fludarabine or 2′-deoxyadenosine, 5-fluorouracil or derivatives thereof), antitumor antibiotics (e.g., mitomycin, adriamycin), plant-derived antitumor agents (e.g., vincristine, vindesine, Taxol), cisplatin, carboplatin, etoposide, TRIMETHOTRIXATE™ (TMTX), TEMOZOLOMIDE™, RALTRITREXED™, S-(4-Nitrobenzyl)-6-thioinosine (NBMPR),6-benzyguanidine (6-BG), bis-chloronitrosourea (BCNU) and CAMPTOTHECIN™, immunomodulating agents such as thalidomide (Thalomid®) Lenalidomide (Revlimid®) Pomalidomide (Pomalyst®), proteasome inhibitors such as Bortezomib (Velcade®), Carfilzomib (Kyprolis®), Histone deacetylase (HDAC) inhibitors such as Panobinostat (Farydak®), or a therapeutic derivative of any thereof.

In a more particular embodiment, said engineered cells of the present invention can advantageously combine an expression of CYP2C9 gene to confer hypersensitivity to cyclosphosphamide and/or isophosphamide and said further genetic engineering being a gene inactivation of a gene selected in the group of deoxycytidine kinase (dCk), hypoxanthine guanine phosphoribosyl transferase (HPRT), glucocorticoid receptor (GR) and CD52, conferring specific drug resistance to purine nucleoside analogues (PNAs)—such as clofarabine or fludarabine—, corticosteroids, alemtuzumab respectively.

Referring to the previous embodiment, said engineered cells of the present invention can advantageously combine an expression of CYP2C9 gene to confer hypersensitivity to cyclosphosphamide and/or isophosphamide and said further genetic engineering is inactivation of dCk gene conferring specific drug resistance to purine nucleoside analogues (PNAs)—such as clofarabine or fludarabine.

As exemplary embodiments, said engineered cells of the present invention can advantageously combine an expression of CYP2C9 gene to confer hypersensitivity to cyclosphosphamide and/or isophosphamide and said further genetic engineering being a gene expression of a mutated gene selected in the group consisting of dihydrofolate reductase (DHFR) inosine monophosphate dehydrogenase 2 (IMPDH2), calcineurin (PP2B) and methylguanine transferase (MGMT), conferring specific drug resistance to respectively anti-folate preferably methotrexate (MTX), to MPDH inhibitors such as mycophenolic acid (MPA) or its prodrug mycophenolate mofetil (MMF), to calcineurin inhibitor such as FK506 and/or Cs and to alkylating agents, such as nitrosoureas and temozolomide (TMZ).

The above mutated genes such as DHFR, IMPDH2, PP2B, MGMT can be obtained such as described in WO 2015075195.

As other exemplary embodiments, said engineered cells of the present invention can advantageously combine an expression of CYP2C9 gene to confer hypersensitivity to cyclosphosphamide and/or isophosphamide and said further genetic engineering being a gene expression of a wild type gene selected in the group consisting of MDR1, ble and mcrA, conferring specific drug resistance to respectively MDR1 resistance drugs such as 4-nitroquinoline-N-oxide, cerulenin, and brefeldin A, to bleomycin and to mitomycin C.

According to another embodiment, said engineered cells of the present invention can advantageously combine an expression of CYP2C9 gene to confer hypersensitivity to cyclosphosphamide and/or isophosphamide and said further genetic engineering is an expression of another gene which confers a supplemental hypersensitivity to another specific drug, preferably one gene selected in the group consisting of CYP2D6-2, CYP3A4, CYP2D6-1, CYP2C19, CYP2B6 and CYP1A2 conferring drug-specific hypersensitivity to cyclophosphamide and/or isophosphamide.

Said method can be used to produce engineered cells for treating cancer, infection or immune disease in a patient by unique or sequential administration thereof to a patient.

As a preferred embodiment, the invention provides the administration of an immune cell made hypersensitive to cyclosphosphamide and/or isophosphamide drug by expressing the CYP2C9 gene, said cell being further engineered to endow a chimeric antigen receptor (CAR) against said cancerous cell, infectious agent or dysfunctioning host immune cell.

Overexpression of CYP3A4 to Confer Hypersensitivity to Cyclosphosphamide and/or Isophosphamide

The immune cells according to the present invention, which CYP3A4 is expressed, are produced to be administered to the patient prior to their elimination by the cyclosphosphamide and/or isophosphamide drug in case of need (such as occurrence of an adverse event). Expression of CYP3A4 has been found by the inventors to confer sensitivity of cells derived from lymphoid progenitor cells, such as NK cells and T cells, to cyclosphosphamide and/or isophosphamide. Thus, to modulate or terminate the treatment, further administration of cyclosphosphamide and/or isophosphamide to which said cells have been made sensitive may be performed in order to deplete in vivo said cells.

According to one embodiment, the present invention relates to a method of producing human cell that may be depleted in-vivo as part of a cell therapy or immunotherapy treatment, said method comprising:

(a) Providing a human cell;

(b) Inducing hypersensitivity to cyclosphosphamide and/or isophosphamide into said cell by selectively expressing or overexpressing at least CYP3A4 transgene involved in the mechanism of action of said drug,

(c) Optionally assaying the hypersensitivity to said drug of said cell engineered in step b);

(d) Expanding said engineered cell obtained in step b).

In another specific embodiment, the gene encoding for the human cytochrome P450 3A4 isoform 1 (CYP3A4) which is used in the present invention to be expressed is the one of SEQ ID NO. 23 (AATCACTGCTGTGCAGGGCAGGAAAGCTCCATGCACATAGCCCAGCAAAGAGCAACACAGAGCTGAAAGGA AGACTCAGAGGAGAGAGATAAGTAAGGAAAGTAGTGATGGCTCTCATCCCAGACTTGGCCATGGAAACCTGG CTTCTCCTGGCTGTCAGCCTGGTGCTCCTCTATCTATATGGAACCCATTCACATGGACTTTTTAAGAAGCTTGGA ATTCCAGGGCCCACACCTCTGCCTTTTTTGGGAAATATTTTGTCCTACCATAAGGGCTTTTGTATGTTTGACATG GAATGTCATAAAAAGTATGGAAAAGTGTGGGGCTTTTATGATGGTCAACAGCCTGTGCTGGCTATCACAGATC CTGACATGATCAAAACAGTGCTAGTGAAAGAATGTTATTCTGTCTTCACAAACCGGAGGCCTTTTGGTCCAGTG GGATTTATGAAAAGTGCCATCTCTATAGCTGAGGATGAAGAATGGAAGAGATTACGATCATTGCTGTCTCCAA CCTTCACCAGTGGAAAACTCAAGGAGATGGTCCCTATCATTGCCCAGTATGGAGATGTGTTGGTGAGAAATCT GAGGCGGGAAGCAGAGACAGGCAAGCCTGTCACCTTGAAAGACGTCTTTGGGGCCTACAGCATGGATGTGAT CACTAGCACATCATTTGGAGTGAACATCGACTCTCTCAACAATCCACAAGACCCCTTTGTGGAAAACACCAAGA AGCTTTTAAGATTTGATTTTTTGGATCCATTCTTTCTCTCAATAACAGTCTTTCCATTCCTCATCCCAATTCTTGAA GTATTAAATATCTGTGTGTTTCCAAGAGAAGTTACAAATTTTTTAAGAAAATCTGTAAAAAGGATGAAAGAAAG TCGCCTCGAAGATACACAAAAGCACCGAGTGGATTTCCTTCAGCTGATGATTGACTCTCAGAATTCAAAAGAAA CTGAGTCCCACAAAGCTCTGTCCGATCTGGAGCTCGTGGCCCAATCAATTATCTTTATTTTTGCTGGCTATGAAA CCACGAGCAGTGTTCTCTCCTTCATTATGTATGAACTGGCCACTCACCCTGATGTCCAGCAGAAACTGCAGGAG GAAATTGATGCAGTTTTACCCAATAAGGCACCACCCACCTATGATACTGTGCTACAGATGGAGTATCTTGACAT GGTGGTGAATGAAACGCTCAGATTATTCCCAATTGCTATGAGACTTGAGAGGGTCTGCAAAAAAGATGTTGAG ATCAATGGGATGTTCATTCCCAAAGGGGTGGTGGTGATGATTCCAAGCTATGCTCTTCACCGTGACCCAAAGT ACTGGACAGAGCCTGAGAAGTTCCTCCCTGAAAGATTCAGCAAGAAGAACAAGGACAACATAGATCCTTACAT ATACACACCCTTTGGAAGTGGACCCAGAAACTGCATTGGCATGAGGTTTGCTCTCATGAACATGAAACTTGCTC TAATCAGAGTCCTTCAGAACTTCTCCTTCAAACCTTGTAAAGAAACACAGATCCCCCTGAAATTAAGCTTAGGA GGACTTCTTCAACCAGAAAAACCCGTTGTTCTAAAGGTTGAGTCAAGGGATGGCACCGTAAGTGGAGCCTGAA TTTTCCTAAGGACTTCTGCTTTGCTCTTCAAGAAATCTGTGCCTGAGAACACCAGAGACCTCAAATTACTTTGTG AATAGAACTCTGAAATGAAGATGGGCTTCATCCAATGGACTGCATAAATAACCGGGGATTCTGTACATGCATT GAGCTCTCTCATTGTCTGTGTAGAGTGTTATACTTGGGAATATAAAGGAGGTGACCAAATCAGTGTGAGGAGG TAGATTTGGCTCCTCTGCTTCTCACGGGACTATTTCCACCACCCCCAGTTAGCACCATTAACTCCTCCTGAGCTCT GATAAGAGAATCAACATTTCTCAATAATTTCCTCCACAAATTATTAATGAAAATAAGAATTATTTTGATGGCTCT AACAATGACATTTATATCACATGTTTTCTCTGGAGTATTCTATAAGTTTTATGTTAAATCAATAAAGACCACTTTA CAAAAGTATTATCAGATGCTTTCCTGCACATTAAGGAGAAATCTATAGAACTGAATGAGAACCAACAAGTAAA TATTTTTGGTCATTGTAATCACTGTTGGCGTGGGGCCTTTGTCAGAACTAGAATTTGATTATTAACATAGGTGA AAGTTAATCCACTGTGACTTTGCCCATTGTTTAGAAAGAATATTCATAGTTTAATTATGCCTTTTTTGATCAGGC ACAGTGGCTCACGCCTGTAATCCTAGCAGTTTGGGAGGCTGAGCCGGGTGGATCGCCTGAGGTCAGGAGTTC AAGACAAGCCTGGCCTACATGGTTGAAACCCCATCTCTACTAAAAATACACAAATTAGCTAGGCATGGTGGAC TCGCCTGTAATCTCACTACACAGGAGGCTGAGGCAGGAGAATCACTTGAACCTGGGAGGCGGATGTTGAAGT GAGCTGAGATTGCACCACTGCACTCCAGTCTGGGTGAGAGTGAGACTCAGTCTTAAAAAAATATGCCTTTTTG AAGCACGTACATTTTGTAACAAAGAACTGAAGCTCTTATTATATTATTAGTTTTGATTTAATGTTTTCAGCCCATC TCCTTTCATATTTCTGGGAGACAGAAAACATGTTTCCCTACACCTCTTGCATTCCATCCTCAACACCCAACTGTCT CGATGCAATGAACACTTAATAAAAAACAGTCGATTGGTCAATTGATTGAGCAATAAGCCT)RefSeq n^{o} NP_059488, or a variant thereof comprising a nucleotide sequence that has at least 60%, such as at least 80%, at least 85%, at least 90% or at least 95%, sequence identity with the nucleotide sequence set forth in SEQ ID NO: 23 over the entire length of SEQ ID NO: 23. However, it is understood that due to the degeneration of the genetic code any other suitable nucleotide sequence coding for the amino acid sequence set forth in SEQ ID NO: 23 is also encompassed by the present disclosure.

Accordingly, in certain embodiment of the present invention, the human cytochrome P450 3A4 isoform 1 to be expressed comprises a polypeptide of SEQ ID NO: 4 (MALIPDLAMETWLLLAVSLVLLYLYGTHSHGLFKKLGIPGPTPLPFLGNILSYHKGFCMFDMECHKKYGKVWGFYD GQQPVLAITDPDMIKTVLVKECYSVFTNRRPFGPVGFMKSAISIAEDEEWKRLRSLLSPTFTSGKLKEMVPIIAQYGD VLVRNLRREAETGKPVTLKDVFGAYSMDVITSTSFGVNIDSLNNPQDPFVENTKKLLRFDFLDPFFLSITVFPFLIPILEV LNICVFPREVTNFLRKSVKRMKESRLEDTQKHRVDFLQLMIDSQNSKETESHKALSDLELVAQSIIFIFAGYETTSSVLS FIMYELATHPDVQQKLQEEIDAVLPNKAPPTYDTVLQMEYLDMVVNETLRLFPIAMRLERVCKKDVEINGMFIPKG VVVMIPSYALHRDPKYWTEPEKFLPERFSKKNKDNIDPYIYTPFGSGPRNCIGMRFALMNMKLALIRVLQNFSFKPC KETQIPLKLSLGGLLQPEKPVVLKVESRDGTVSGA), corresponding to P08684 (Ref Uniprot), or a variant thereof comprising an amino acid sequence that has at least 60%, such as at least 80%, at least 85%, at least 90% or at least 95%, sequence identity with the amino acid sequence set forth in SEQ ID NO: 4 over the entire length of SEQ ID NO: 4. The variant may comprise an amino acid sequence which has one or more, such as two, three, four, five or six amino acid substitutions compared to SEQ ID NO: 4. Preferably, such amino acid substitution is a conservative substitution which means that one amino acid is replaced by another one that is similar in size and chemical properties. Such conservative amino acid substitution may thus have minor effects on the peptide structure and can thus be tolerated without compromising function. Preferably, such variant is capable of maintaining the activity of human cytochrome P450 3A4 isoform 1 and is capable of oxidizing small foreign organic molecules (xenobiotics), such as toxins or drugs.

In a particular embodiment, said engineered cells of the present invention can advantageously combine an expression of CYP3A4 gene to confer hypersensitivity to cyclosphosphamide and/or isophosphamide and said and a further genetic engineering to confer specific resistance to another drug, such additional modification may be performed by a gene inactivation or by overexpression of a wild-type or a mutant form of a gene, said gene being involved in the metabolization of said second drug.

In another particular embodiment, said further genetic engineered of cells according to the present invention, in addition to the gene expression of CYP3A4, confers resistance to said second drug selected in the group consisting of alkylating agents (other than cyclophosphamide and isophosphamide), metabolic antagonists (e.g., purine nucleoside antimetabolite such as clofarabine, fludarabine or 2′-deoxyadenosine, 5-fluorouracil or derivatives thereof), antitumor antibiotics (e.g., mitomycin, adriamycin), plant-derived antitumor agents (e.g., vincristine, vindesine, Taxol), cisplatin, carboplatin, etoposide, TRIMETHOTRIXATE™ (TMTX), TEMOZOLOMIDE™, RALTRITREXED™, S-(4-Nitrobenzyl)-6-thioinosine (NBMPR),6-benzyguanidine (6-BG), bis-chloronitrosourea (BCNU) and CAMPTOTHECIN™, immunomodulating agents such as thalidomide (Thalomid®) Lenalidomide (Revlimid®) Pomalidomide (Pomalyst®), proteasome inhibitors such as Bortezomib (Velcade®), Carfilzomib (Kyprolis®), Histone deacetylase (HDAC) inhibitors such as Panobinostat (Farydak®), or a therapeutic derivative of any thereof.

In a more particular embodiment, said engineered cells of the present invention can advantageously combine an expression of CYP3A4 gene to confer hypersensitivity to cyclosphosphamide and/or isophosphamide and said further genetic engineering being a gene inactivation of a gene selected in the group of deoxycytidine kinase (dCk), hypoxanthine guanine phosphoribosyl transferase (HPRT), glucocorticoid receptor (GR) and CD52, conferring specific drug resistance to purine nucleoside analogues (PNAs)—such as clofarabine or fludarabine—, corticosteroids, alemtuzumab respectively.

Referring to the previous embodiment, said engineered cells of the present invention can advantageously combine an expression of CYP3A4 gene to confer hypersensitivity to cyclosphosphamide and/or isophosphamide and said further genetic engineering is inactivation of dCk gene conferring specific drug resistance to purine nucleoside analogues (PNAs)—such as clofarabine or fludarabine.

As exemplary embodiments, said engineered cells of the present invention can advantageously combine an expression of CYP3A4 gene to confer hypersensitivity to cyclosphosphamide and/or isophosphamide and said further genetic engineering being a gene expression of a mutated gene selected in the group consisting of dihydrofolate reductase (DHFR) inosine monophosphate dehydrogenase 2 (IMPDH2), calcineurin (PP2B) and methylguanine transferase (MGMT), conferring specific drug resistance to respectively anti-folate preferably methotrexate (MTX), to MPDH inhibitors such as mycophenolic acid (MPA) or its prodrug mycophenolate mofetil (MMF), to calcineurin inhibitor such as FK506 and/or Cs and to alkylating agents, such as nitrosoureas and temozolomide (TMZ).

The above mutated genes such as DHFR, IMPDH2, PP2B, MGMT can be obtained such as described in WO 2015075195.

As other exemplary embodiments, said engineered cells of the present invention can advantageously combine an expression of CYP3A4 gene to confer hypersensitivity to cyclosphosphamide and/or isophosphamide and said further genetic engineering being a gene expression of a wild type gene selected in the group consisting of MDR1, ble and mcrA, conferring specific drug resistance to respectively MDR1 resistance drugs such as 4-nitroquinoline-N-oxide, cerulenin, and brefeldin A, to bleomycin and to mitomycin C.

According to another embodiment, said engineered cells of the present invention can advantageously combine an expression of CYP3A4 gene to confer hypersensitivity to cyclosphosphamide and/or isophosphamide and said further genetic engineering is an expression of another gene which confers a supplemental hypersensitivity to another specific drug, preferably one gene selected in the group consisting of CYP2D6-2, CDA, CYP2C9, CYP2D6-1, CYP2C19, CYP2B6 and CYP1A2 conferring drug-specific hypersensitivity to cyclophosphamide and/or isophosphamide.

Said method can be used to produce engineered cells for treating cancer, infection or immune disease in a patient by unique or sequential administration thereof to a patient.

As a preferred embodiment, the invention provides the administration of an immune cell made hypersensitive to cyclosphosphamide and/or isophosphamide drug by expressing the CYP3A4 gene, said cell being further engineered to endow a chimeric antigen receptor (CAR) against said cancerous cell, infectious agent or dysfunctioning host immune cell.

Overexpression of CYP2D6-1 to Confer Hypersensitivity to Cyclosphosphamide and/or Isophosphamide

The immune cells according to the present invention, which CYP2D6-1 is expressed, are produced to be administered to the patient prior to their elimination by the cyclosphosphamide and/or isophosphamide drug in case of need (such as occurrence of an adverse event). Expression of CYP2D6-1 has been found by the inventors to confer sensitivity of cells derived from lymphoid progenitor cells, such as NK cells and T cells, to cyclosphosphamide and/or isophosphamide. Thus, to modulate or terminate the treatment, further administration of cyclosphosphamide and/or isophosphamide to which said cells have been made sensitive may be performed in order to deplete in vivo said cells.

According to one embodiment, the present invention relates to a method of producing human cell that may be depleted in-vivo as part of a cell therapy or immunotherapy treatment, said method comprising:

(a) Providing a human cell;

(b) Inducing hypersensitivity to cyclosphosphamide and/or isophosphamide into said cell by selectively expressing or overexpressing at least CYP2D6-1 transgene involved in the mechanism of action of said drug,

(c) Optionally assaying the hypersensitivity to said drug of said cell engineered in step b);

(d) Expanding said engineered cell obtained in step b).

In a specific embodiment, the gene encoding for the human cytochrome P450 2D6 isoform 1 which is used in the present invention to be expressed is the one of SEQ ID NO. 21 (CAAACCATGGGAGGCTCCTCTCCTAGACCCTGCATCCTGAAAGCTGCGTACCTGAGAGCCTGCGGTCTGGCTG CAGGGACACACCCAAGGGGAGGAGCTGCAATCGTGTCTGGGGCCCCAGCCCAGGCTGGCCGGAGCTCCTGTT TCCCGCTGCTCTGCTGCCTGCCCGGGGTACCAACATGGCCCAGAAGCGTCCTGCCTGCACCCTGAAGCCTGAG TGTGTCCAGCAGCTGCTGGTTTGCTCCCAGGAGGCCAAGAAGTCAGCCTACTGCCCCTACAGTCACTTTCCTGT GGGGGCTGCCCTGCTCACCCAGGAGGGGAGAATCTTCAAAGGGTGCAACATAGAAAATGCCTGCTACCCGCT GGGCATCTGTGCTGAACGGACCGCTATCCAGAAGGCCGTCTCAGAAGGGTACAAGGATTTCAGGGCAATTGC TATCGCCAGTGACATGCAAGATGATTTTATCTCTCCATGTGGGGCCTGCAGGCAAGTCATGAGAGAGTTTGGC ACCAACTGGCCCGTGTACATGACCAAGCCGGATGGTACGTATATTGTCATGACGGTCCAGGAGCTGCTGCCCT CCTCCTTTGGGCCTGAGGACCTGCAGAAGACCCAGTGACAGCCAGAGAATGCCCACTGCCTGTAACAGCCACC TGGAGAACTTCATAAAGATGTCTCACAGCCCTGGGGACACCTGCCCAGTGGGCCCCAGCCCTACAGGGACTGG GCAAAGATGATGTTTCCAGATTACACTCCAGCCTGAGTCAGCACCCCTCCTAGCAACCTGCCTTGGGACTTAGA ACACCGCCGCCCCCTGCCCCACCTTTCCTTTCCTTCCTGTGGGCCCTCTTTCAAAGTCCAGCCTAGTCTGGACTG CTTCCCCATCAGCCTTCCCAAGGTTCTATCCTGTTCCGAGCAACTTTTCTAATTATAAACATCACAGAACATCCTG GATC)RefSeq n^{o} NM_000106.5 or a variant thereof comprising a nucleotide sequence that has at least 60%, such as at least 80%, at least 85%, at least 90% or at least 95%, sequence identity with the nucleotide sequence set forth in SEQ ID NO: 21 over the entire length of SEQ ID NO: 21. However, it is understood that due to the degeneration of the genetic code any other suitable nucleotide sequence coding for the amino acid sequence set forth in SEQ ID NO: 21 is also encompassed by the present disclosure.

In another specific embodiment, said human cytochrome P450 2D6 isoform 1 to be expressed comprises a polypeptide of SEQ ID NO: 5 (MGLEALVPLAVIVAIFLLLVDLMHRRQRWAARYPPGPLPLPGLGNLLHVDFQNTPYCFDQLRRRFGDVFS LQLAWTPVVVLNGLAAVREALVTHGEDTADRPPVPITQILGFGPRSQGVFLARYGPAWREQRRFSVSTLRNLGLGK KSLEQWVTEEAACLCAAFANHSGRPFRPNGLLDKAVSNVIASLTCGRRFEYDDPRFLRLLDLAQEGLKEESGFLREVL NAVPVLLHIPALAGKVLRFQKAFLTQLDELLTEHRMTWDPAQPPRDLTEAFLAEMEKAKGNPESSFNDENLCIVVA DLFSAGMVTTSTTLAWGLLLMILHPDVQRRVQQEIDDVIGQVRRPEMGDQAHMPYTTAVIHEVQRFGDIVPLGV THMTSRDIEVQGFRIPKGTTLITNLSSVLKDEAVWEKPFRFHPEHFLDAQGHFVKPEAFLPFSAGRRACLGEPLARM ELFLFFTSLLQHFSFSVPTGQPRPSHHGVFAFLVTPSPYELCAVPR), corresponding to P10635 (Ref Uniprot), or a variant thereof comprising an nucleotide sequence that has at least 60%, such as at least 80%, at least 85%, at least 90% or at least 95%, sequence identity with nucleotide sequence set forth in SEQ ID NO: 5 over the entire length of SEQ ID NO: 5. The variant may comprise an amino acid sequence which has one or more, such as two, three, four, five or six amino acid substitutions compared to SEQ ID NO: 5. Preferably, such amino acid substitution is a conservative substitution which means that one amino acid is replaced by another one that is similar in size and chemical properties. Such conservative amino acid substitution may thus have minor effects on the peptide structure and can thus be tolerated without compromising function. Preferably, such variant is capable of maintaining the activity of cytochrome P450 2D6 isoform 1 and is capable of metabolizing and eliminating of clinically used drugs, in a process referred to as 0-demethylation.

In a particular embodiment, said engineered cells of the present invention can advantageously combine an expression of CYP2D6-1 gene to confer hypersensitivity to cyclosphosphamide and/or isophosphamide and said and a further genetic engineering to confer specific resistance to another drug, such additional modification may be performed by a gene inactivation or by overexpression of a wild-type or a mutant form of a gene, said gene being involved in the metabolization of said second drug.

In another particular embodiment, said further genetic engineered of cells according to the present invention, in addition to the gene expression of CYP2D6-1, confers resistance to said second drug selected in the group consisting of alkylating agents (other than cyclophosphamide and isophosphamide), metabolic antagonists (e.g., purine nucleoside antimetabolite such as clofarabine, fludarabine or 2′-deoxyadenosine, 5-fluorouracil or derivatives thereof), antitumor antibiotics (e.g., mitomycin, adriamycin), plant-derived antitumor agents (e.g., vincristine, vindesine, Taxol), cisplatin, carboplatin, etoposide, TRIMETHOTRIXATE™ (TMTX), TEMOZOLOMIDE™, RALTRITREXED™, S-(4-Nitrobenzyl)-6-thioinosine (NBMPR),6-benzyguanidine (6-BG), bis-chloronitrosourea (BCNU) and CAMPTOTHECIN™, immunomodulating agents such as thalidomide (Thalomid®) Lenalidomide (Revlimid®) Pomalidomide (Pomalyst®), proteasome inhibitors such as Bortezomib (Velcade®), Carfilzomib (Kyprolis®), Histone deacetylase (HDAC) inhibitors such as Panobinostat (Farydak®), or a therapeutic derivative of any thereof.

In a more particular embodiment, said engineered cells of the present invention can advantageously combine an expression of CYP2D6-1 gene to confer hypersensitivity to cyclosphosphamide and/or isophosphamide and said further genetic engineering being a gene inactivation of a gene selected in the group of deoxycytidine kinase (dCk), hypoxanthine guanine phosphoribosyl transferase (HPRT), glucocorticoid receptor (GR) and CD52, conferring specific drug resistance to purine nucleoside analogues (PNAs)—such as clofarabine or fludarabine—, corticosteroids, alemtuzumab respectively.

Referring to the previous embodiment, said engineered cells of the present invention can advantageously combine an expression of CYP2D6-1 gene to confer hypersensitivity to cyclosphosphamide and/or isophosphamide and said further genetic engineering is inactivation of dCk gene conferring specific drug resistance to purine nucleoside analogues (PNAs)—such as clofarabine or fludarabine.

As exemplary embodiments, said engineered cells of the present invention can advantageously combine an expression of CYP2D6-1 gene to confer hypersensitivity to cyclosphosphamide and/or isophosphamide and said further genetic engineering being a gene expression of a mutated gene selected in the group consisting of dihydrofolate reductase (DHFR) inosine monophosphate dehydrogenase 2 (IMPDH2), calcineurin (PP2B) and methylguanine transferase (MGMT), conferring specific drug resistance to respectively anti-folate preferably methotrexate (MTX), to MPDH inhibitors such as mycophenolic acid (MPA) or its prodrug mycophenolate mofetil (MMF), to calcineurin inhibitor such as FK506 and/or Cs and to alkylating agents, such as nitrosoureas and temozolomide (TMZ).

The above mutated genes such as DHFR, IMPDH2, PP2B, MGMT can be obtained such as described in WO 2015075195.

As other exemplary embodiments, said engineered cells of the present invention can advantageously combine an expression of CYP2D6-1 gene to confer hypersensitivity to cyclosphosphamide and/or isophosphamide and said further genetic engineering being a gene expression of a wild type gene selected in the group consisting of MDR1, ble and mcrA, conferring specific drug resistance to respectively MDR1 resistance drugs such as 4-nitroquinoline-N-oxide, cerulenin, and brefeldin A, to bleomycin and to mitomycin C.

According to another embodiment, said engineered cells of the present invention can advantageously combine an expression of CYP2D6-1 gene to confer hypersensitivity to cyclosphosphamide and/or isophosphamide and said further genetic engineering is an expression of another gene which confers a supplemental hypersensitivity to another specific drug, preferably one gene selected in the group consisting of CYP2D6-2, CDA, CYP2C9, CYP3A4, CYP2C19, CYP2B6 and CYP1A2 conferring drug-specific hypersensitivity to cyclophosphamide and/or isophosphamide.

Said method can be used to produce engineered cells for treating cancer, infection or immune disease in a patient by unique or sequential administration thereof to a patient.

As a preferred embodiment, the invention provides the administration of an immune cell made hypersensitive to cyclosphosphamide and/or isophosphamide drug by expressing the CYP2D6-1 gene, said cell being further engineered to endow a chimeric antigen receptor (CAR) against said cancerous cell, infectious agent or dysfunctioning host immune cell.

Overexpression of CYP2C19 to Confer Hypersensitivity to Cyclosphosphamide and/or Isophosphamide

The immune cells according to the present invention, which CYP2C19 is expressed, are produced to be administered to the patient prior to their elimination by the cyclosphosphamide and/or isophosphamide drug in case of need (such as occurrence of an adverse event). Expression of CYP2C19 has been found by the inventors to confer sensitivity of cells derived from lymphoid progenitor cells, such as NK cells and T cells, to cyclosphosphamide and/or isophosphamide. Thus, to modulate or terminate the treatment, further administration of cyclosphosphamide and/or isophosphamide to which said cells have been made sensitive may be performed in order to deplete in vivo said cells.

According to one embodiment, the present invention relates to a method of producing human cell that may be depleted in-vivo as part of a cell therapy or immunotherapy treatment, said method comprising:

(a) Providing a human cell;

(b) Inducing hypersensitivity to cyclosphosphamide and/or isophosphamide into said cell by selectively expressing or overexpressing at least CYP2C19 transgene involved in the mechanism of action of said drug,

(c) Optionally assaying the hypersensitivity to said drug of said cell engineered in step b);

(d) Expanding said engineered cell obtained in step b).

In another specific embodiment, the gene encoding for the human cytochrome P450 2C19 precursor (CYP2C19) which is used in the present invention to be expressed is the one of SEQ ID NO. 25 (GTCTTAACAAGAGGAGAAGGCTTCAATGGATCCTTTTGTGGTCCTTGTGCTCTGTCTCTCATGTTTGCTTCTCCT TTCAATCTGGAGACAGAGCTCTGGGAGAGGAAAACTCCCTCCTGGCCCCACTCCTCTCCCAGTGATTGGAAAT ATCCTACAGATAGATATTAAGGATGTCAGCAAATCCTTAACCAATCTCTCAAAAATCTATGGCCCTGTGTTCACT CTGTATTTTGGCCTGGAACGCATGGTGGTGCTGCATGGATATGAAGTGGTGAAGGAAGCCCTGATTGATCTTG GAGAGGAGTTTTCTGGAAGAGGCCATTTCCCACTGGCTGAAAGAGCTAACAGAGGATTTGGAATCGTTTTCAG CAATGGAAAGAGATGGAAGGAGATCCGGCGTTTCTCCCTCATGACGCTGCGGAATTTTGGGATGGGGAAGAG GAGCATTGAGGACCGTGTTCAAGAGGAAGCCCGCTGCCTTGTGGAGGAGTTGAGAAAAACCAAGGCTTCACC CTGTGATCCCACTTTCATCCTGGGCTGTGCTCCCTGCAATGTGATCTGCTCCATTATTTTCCAGAAACGTTTCGA TTATAAAGATCAGCAATTTCTTAACTTGATGGAAAAATTGAATGAAAACATCAGGATTGTAAGCACCCCCTGGA TCCAGATATGCAATAATTTTCCCACTATCATTGATTATTTCCCGGGAACCCATAACAAATTACTTAAAAACCTTG CTTTTATGGAAAGTGATATTTTGGAGAAAGTAAAAGAACACCAAGAATCGATGGACATCAACAACCCTCGGGA CTTTATTGATTGCTTCCTGATCAAAATGGAGAAGGAAAAGCAAAACCAACAGTCTGAATTCACTATTGAAAACT TGGTAATCACTGCAGCTGACTTACTTGGAGCTGGGACAGAGACAACAAGCACAACCCTGAGATATGCTCTCCT TCTCCTGCTGAAGCACCCAGAGGTCACAGCTAAAGTCCAGGAAGAGATTGAACGTGTCATTGGCAGAAACCG GAGCCCCTGCATGCAGGACAGGGGCCACATGCCCTACACAGATGCTGTGGTGCACGAGGTCCAGAGATACAT CGACCTCATCCCCACCAGCCTGCCCCATGCAGTGACCTGTGACGTTAAATTCAGAAACTACCTCATTCCCAAGG GCACAACCATATTAACTTCCCTCACTTCTGTGCTACATGACAACAAAGAATTTCCCAACCCAGAGATGTTTGACC CTCGTCACTTTCTGGATGAAGGTGGAAATTTTAAGAAAAGTAACTACTTCATGCCTTTCTCAGCAGGAAAACGG ATTTGTGTGGGAGAGGGCCTGGCCCGCATGGAGCTGTTTTTATTCCTGACCTTCATTTTACAGAACTTTAACCT GAAATCTCTGATTGACCCAAAGGACCTTGACACAACTCCTGTTGTCAATGGATTTGCTTCTGTCCCGCCCTTCTA TCAGCTGTGCTTCATTCCTGTCTGAAGAAGCACAGATGGTCTGGCTGCTCCTGTGCTGTCCCTGCAGCTCTCTTT CCTCTGGTCCAAATTTCACTATCTGTGATGCTTCTTCTGACCCGTCATCTCACATTTTCCCTTCCCCCAAGATCTA GTGAACATTCAGCCTCCATTAAAAAAGTTTCACTGTGCAAATATATCTGCTATTCCCCATACTCTATAATAGTTA CATTGAGTGCCACATAATGCTGATACTTGTCTAATGTTGAGTTATTAACATATTATTATTAAATAGAGAAAGAT GATTTGTGTATTAT)RefSeq n^{o} NP_000760, or a variant thereof comprising an nucleotide sequence that has at least 60%, such as at least 80%, at least 85%, at least 90% or at least 95%, sequence identity with the nucleotide sequence set forth in SEQ ID NO: 25 over the entire length of SEQ ID NO: 25. However, it is understood that due to the degeneration of the genetic code any other suitable nucleotide sequence coding for the amino acid sequence set forth in SEQ ID NO: 25 is also encompassed by the present disclosure.

Accordingly, in certain embodiment of the present invention, the human cytochrome P450 2C19 precursor to be expressed comprises a polypeptide of SEQ ID NO: 6 (MDPFVVLVLCLSCLLLLSIWRQSSGRGKLPPGPTPLPVIGNILQIDIKDVSKSLTNLSKIYGPVFTLYFGLERMVVLHGY EVVKEALIDLGEEFSGRGHFPLAERANRGFGIVFSNGKRWKEIRRFSLMTLRNFGMGKRSIEDRVQEEARCLVEELR KTKASPCDPTFILGCAPCNVICSIIFQKRFDYKDQQFLNLMEKLNENIRIVSTPWIQICNNFPTIIDYFPGTHNKLLKNL AFMESDILEKVKEHQESMDINNPRDFIDCFLIKMEKEKQNQQSEFTIENLVITAADLLGAGTETTSTTLRYALLLLLKH PEVTAKVQEEIERVIGRNRSPCMQDRGHMPYTDAVVHEVQRYIDLIPTSLPHAVTCDVKFRNYLIPKGTTILTSLTSVL HDNKEFPNPEMFDPRHFLDEGGNFKKSNYFMPFSAGKRICVGEGLARMELFLFLTFILQNFNLKSLIDPKDLDTTPV VNGFASVPPFYQLCFIPV), corresponding to P33261 (Ref Uniprot), or a variant thereof comprising an nucleotide sequence that has at least 60%, such as at least 80%, at least 85%, at least 90% or at least 95%, sequence identity with the nucleotide sequence set forth in SEQ ID NO: 2 over the entire length of SEQ ID NO: 2. The variant may comprise an amino acid sequence which has one or more, such as two, three, four, five or six amino acid substitutions compared to SEQ ID NO: 2.

In a particular embodiment, said engineered cells of the present invention can advantageously combine an expression of CYP2C19 gene to confer hypersensitivity to cyclosphosphamide and/or isophosphamide and said and a further genetic engineering to confer specific resistance to another drug, such additional modification may be performed by a gene inactivation or by overexpression of a wild-type or a mutant form of a gene, said gene being involved in the metabolization of said second drug.

In another particular embodiment, said further genetic engineered of cells according to the present invention, in addition to the gene expression of CYP2C19, confers resistance to said second drug selected in the group consisting of alkylating agents (other than cyclophosphamide and isophosphamide), metabolic antagonists (e.g., purine nucleoside antimetabolite such as clofarabine, fludarabine or 2′-deoxyadenosine, 5-fluorouracil or derivatives thereof), antitumor antibiotics (e.g., mitomycin, adriamycin), plant-derived antitumor agents (e.g., vincristine, vindesine, Taxol), cisplatin, carboplatin, etoposide, TRIMETHOTRIXATE™ (TMTX), TEMOZOLOMIDE™, RALTRITREXED™, S-(4-Nitrobenzyl)-6-thioinosine (NBMPR),6-benzyguanidine (6-BG), bis-chloronitrosourea (BCNU) and CAMPTOTHECIN™, immunomodulating agents such as thalidomide (Thalomid®) Lenalidomide (Revlimid®) Pomalidomide (Pomalyst®), proteasome inhibitors such as Bortezomib (Velcade®), Carfilzomib (Kyprolis®), Histone deacetylase (HDAC) inhibitors such as Panobinostat (Farydak®), or a therapeutic derivative of any thereof.

In a more particular embodiment, said engineered cells of the present invention can advantageously combine an expression of CYP2C19 gene to confer hypersensitivity to cyclosphosphamide and/or isophosphamide and said further genetic engineering being a gene inactivation of a gene selected in the group of deoxycytidine kinase (dCk), hypoxanthine guanine phosphoribosyl transferase (HPRT), glucocorticoid receptor (GR) and CD52, conferring specific drug resistance to purine nucleoside analogues (PNAs)—such as clofarabine or fludarabine—, corticosteroids, alemtuzumab respectively.

Referring to the previous embodiment, said engineered cells of the present invention can advantageously combine an expression of CYP2C19 gene to confer hypersensitivity to cyclosphosphamide and/or isophosphamide and said further genetic engineering is inactivation of dCk gene conferring specific drug resistance to purine nucleoside analogues (PNAs)—such as clofarabine or fludarabine.

As exemplary embodiments, said engineered cells of the present invention can advantageously combine an expression of CYP2C19 gene to confer hypersensitivity to cyclosphosphamide and/or isophosphamide and said further genetic engineering being a gene expression of a mutated gene selected in the group consisting of dihydrofolate reductase (DHFR) inosine monophosphate dehydrogenase 2 (IMPDH2), calcineurin (PP2B) and methylguanine transferase (MGMT), conferring specific drug resistance to respectively anti-folate preferably methotrexate (MTX), to MPDH inhibitors such as mycophenolic acid (MPA) or its prodrug mycophenolate mofetil (MMF), to calcineurin inhibitor such as FK506 and/or Cs and to alkylating agents, such as nitrosoureas and temozolomide (TMZ).

The above mutated genes such as DHFR, IMPDH2, PP2B, MGMT can be obtained such as described in WO 2015075195.

As other exemplary embodiments, said engineered cells of the present invention can advantageously combine an expression of CYP2C19 gene to confer hypersensitivity to cyclosphosphamide and/or isophosphamide and said further genetic engineering being a gene expression of a wild type gene selected in the group consisting of MDR1, ble and mcrA, conferring specific drug resistance to respectively MDR1 resistance drugs such as 4-nitroquinoline-N-oxide, cerulenin, and brefeldin A, to bleomycin and to mitomycin C.

According to another embodiment, said engineered cells of the present invention can advantageously combine an expression of CYP2C19 gene to confer hypersensitivity to cyclosphosphamide and/or isophosphamide and said further genetic engineering is an expression of another gene which confers a supplemental hypersensitivity to another specific drug, preferably one gene selected in the group consisting of CYP2D6-2, CDA, CYP2C9, CYP3A4, CYP2D6-1, CYP2B6 and CYP1A2 conferring drug-specific hypersensitivity to cyclophosphamide and/or isophosphamide.

Said method can be used to produce engineered cells for treating cancer, infection or immune disease in a patient by unique or sequential administration thereof to a patient.

As a preferred embodiment, the invention provides the administration of an immune cell made hypersensitive to cyclosphosphamide and/or isophosphamide drug by expressing the CYP2C19 gene, said cell being further engineered to endow a chimeric antigen receptor (CAR) against said cancerous cell, infectious agent or dysfunctioning host immune cell.

Overexpression of CYP2B6 to Confer Hypersensitivity to Cyclosphosphamide and/or Isophosphamide

The immune cells according to the present invention, which CYP2B6 is expressed, are produced to be administered to the patient prior to their elimination by the cyclosphosphamide and/or isophosphamide drug in case of need (such as occurrence of an adverse event). Expression of CYP2B6 has been found by the inventors to confer sensitivity of cells derived from lymphoid progenitor cells, such as NK cells and T cells, to cyclosphosphamide and/or isophosphamide. Thus, to modulate or terminate the treatment, further administration of cyclosphosphamide and/or isophosphamide to which said cells have been made sensitive may be performed in order to deplete in vivo said cells.

According to one embodiment, the present invention relates to a method of producing human cell that may be depleted in-vivo as part of a cell therapy or immunotherapy treatment, said method comprising:

(a) Providing a human cell;

(b) Inducing hypersensitivity to cyclosphosphamide and/or isophosphamide into said cell by selectively expressing or overexpressing at least CYP2B6 transgene involved in the mechanism of action of said drug,

(c) Optionally assaying the hypersensitivity to said drug of said cell engineered in step b);

(d) Expanding said engineered cell obtained in step b).

In another specific embodiment, the gene encoding for the human cytochrome P450 2B6 (CYP2B6) which is used in the present invention to be expressed is the one of SEQ ID NO. 27 (GCGGAGCGCGCACGCGGGAACCCGCGCTGGAGGCGGGCGAGGGCCGAGGGGCAGCTAGGGAGCGCGGCT TGAGGAGGGCGGGGCCGCCCCGCAGGCCCGCCAGTGTCCTCAGCTGCCTCCGCGCGCCAAAGTCAAACCCCG ACACCCGCCGGCGGGCCGGTGAGCTCACTAGCTGACCCGGCAGGTCAGGATCTGGCTTAGCGGCGCCGCGAG CTCCAGTGCGCGCACCCGTGGCCGCCTCCCAGCCCTCTTTGCCGGACGAGCTCTGGGCCGCCACAAGACTAAG GAATGGCCACCCCGCCCAAGAGAAGCTGCCCGTCTTTCTCAGCCAGCTCTGAGGGGACCCGCATCAAGAAAAT CTCCATCGAAGGGAACATCGCTGCAGGGAAGTCAACATTTGTGAATATCCTTAAACAATTGTGTGAAGATTGG GAAGTGGTTCCTGAACCTGTTGCCAGATGGTGCAATGTTCAAAGTACTCAAGATGAATTTGAGGAACTTACAA TGTCTCAGAAAAATGGTGGGAATGTTCTTCAGATGATGTATGAGAAACCTGAACGATGGTCTTTTACCTTCCAA ACATATGCCTGTCTCAGTCGAATAAGAGCTCAGCTTGCCTCTCTGAATGGCAAGCTCAAAGATGCAGAGAAAC CTGTATTATTTTTTGAACGATCTGTGTATAGTGACAGGTATATTTTTGCATCTAATTTGTATGAATCTGAATGCA TGAATGAGACAGAGTGGACAATTTATCAAGACTGGCATGACTGGATGAATAACCAATTTGGCCAAAGCCTTGA ATTGGATGGAATCATTTATCTTCAAGCCACTCCAGAGACATGCTTACATAGAATATATTTACGGGGAAGAAATG AAGAGCAAGGCATTCCTCTTGAATATTTAGAGAAGCTTCATTATAAACATGAAAGCTGGCTCCTGCATAGGAC ACTGAAAACCAACTTCGATTATCTTCAAGAGGTGCCTATCTTAACACTGGATGTTAATGAAGACTTTAAAGACA AATATGAAAGTCTGGTTGAAAAGGTCAAAGAGTTTTTGAGTACTTTGTGATCTTGCTGAAGACTACAGGCAGC CAAATGGTTCCAGATACTTCAGCTTTGTGTATCTTCGTAACTTCATATTAATATAAGTTTCTTTAGAAAACCCAA GTTTTTAATCGTTTTTGTTTTAAGGAAAAAAGATTTTTAAAATGAATCTTATGCAAAACTTTTTGACCAGTTTCTT TTCTTTTGTTTTTTTTTTAAAAAAGACATTTAAAGACAAAGACATTATTTCTCATAGCAGGAAATGTAGAGGTAG ATGGTTCCAGTATCAGCATAGTGACTAAACTACATTATAAAAGATCCAGCTTCCTTCTGTCATTCCCCTCTTTTGT CTTCCTCAGCAGGTTGGCTTTTTTCCCTGGTGCCTCTCACTTCGTTGGTGACCAGTTTCTTAAACTGAAAGCTTTA ATGTTACATAGTAAATGGTAGTGTGTCCTGTGTAAATTAGTGTACCTATTAAAAGTTGCAAAGTGGAATTAAAG GAATCCCTAGAATAAGGATTCTGAAGTTTTATTTTAAATTATTATCTTCTTAACAGTTTAGTCCCACCTCTTACTT CCTGCCTCAGTCTGCTTTCTCTACTGTCTGGATTAATTAGGCAGCCTGCTATAAAGTTAAAGTCACACATTTCTA TTTTGCAAACACTGTGATTACTCTTTGCTTTGTAGTTTGCTTTGCTTTGTAGGGTTCTGCTTTTAAGTTTTTCTCTT TTTCAGACAAATTACTGATAAAAATGATATTGCTCTATATGTAATATATCCTGAAAGCATTATTTTTTGTTGAAT AGGAAATAAAATTAATGAAGACAGAGGCTAGAAAGCATCCATTAATTAATGAGACACACTTAACTACTTATCTC TAAACCATCTATGTGAATATTTGTAAAAATAATGAATGGACTCATCTTAGTTCTGTATATAAATATATTTTCTTTC TAGTTTGTTTAGTTAAGGTGTGCAGTGTTTTTCCTGTGTATTAAACCTTTCCATTTTACGTTTTAGAAAATTTTAT GTATTTTAAAATAAGGGGAAGAGTCATTTTCACTTTTAAACTACTATTTTTCTTTCCAAGTCATTTTTGTTTTTGG TTTCTTATTCAAAGATGATAATTTAGTGGATTAACCAGTCCAGACGCACTGATCTTTGCAAAGGAGACTTAATTT CAAATCTGTAATTACCATACATAAACTGTCTCATTATACGTATGCATTTTTTTAGTTTGTTTTTGTTTGGTATAAA TTAATTTGTTAATTAAATATTTCTTAAGTATAAACCTTATGAACTACAGTGGAGCTACACTCATTGAAATGTAAT TTCAGTTCTAAAAAGATGTAATAATCATTTTAGAATTAAAATTTATTCTACTTTTAAATAAATTATGAATATTAAA GGTGAAAATTGTATAAATTACTTTGATTCCATTTTAAGTGGAGACATATTTCAGTGATTTTTAGTAACCTTTAAA AATGTATAATGACTTTTAAAATTTGTAGAATTGAAAAGACGCTAATAAAAATTTATTATTTATTTGTCATGACTC) RefSeq n^{o} NP_000779, or a variant thereof comprising a nucleotide sequence that has at least 60%, such as at least 80%, at least 85%, at least 90% or at least 95%, sequence identity with the nucleotide sequence set forth in SEQ ID NO: 27 over the entire length of SEQ ID NO: 27. However, it is understood that due to the degeneration of the genetic code any other suitable nucleotide sequence coding for the amino acid sequence set forth in SEQ ID NO: 27 is also encompassed by the present disclosure.

Accordingly, in certain embodiment of the present invention, the human cytochrome P450 CYP2B6 to be expressed comprises a polypeptide of SEQ ID NO: 8 (MELSVLLFLALLTGLLLLLVQRHPNTHDRLPPGPRPLPLLGNLLQMDRRGLLKSFLRFREKYGDVFTVHLGPRPVVM LCGVEAIREALVDKAEAFSGRGKIAMVDPFFRGYGVIFANGNRWKVLRRFSVTTMRDFGMGKRSVEERIQEEAQC LIEELRKSKGALMDPTFLFQSITANIICSIVFGKRFHYQDQEFLKMLNLFYQTFSLISSVFGQLFELFSGFLKYFPGAHRQ VYKNLQEINAYIGHSVEKHRETLDPSAPKDLIDTYLLHMEKEKSNAHSEFSHQNLNLNTLSLFFAGTETTSTTLRYGFLL MLKYPHVAERVYREIEQVIGPHRPPELHDRAKMPYTEAVIYEIQRFSDLLPMGVPHIVTQHTSFRGYIIPKDTEVFLILS TALHDPHYFEKPDAFNPDHFLDANGALKKTEAFIPFSLGKRICLGEGIARAELFLFFTTILQNFSMASPVAPEDIDLTP QECGVGKIPPTYQIRFLPR), corresponding to P20813 (Ref Uniprot), or a variant thereof comprising an amino acid sequence that has at least 60%, such as at least 80%, at least 85%, at least 90% or at least 95%, sequence identity with the amino acid sequence set forth in SEQ ID NO: 8 over the entire length of SEQ ID NO: 8. The variant may comprise an amino acid sequence which has one or more, such as two, three, four, five or six amino acid substitutions compared to SEQ ID NO: 8.

Preferably, in all above embodiments, such amino acid substitution is a conservative substitution which means that one amino acid is replaced by another one that is similar in size and chemical properties. Such conservative amino acid substitution may thus have minor effects on the peptide structure and can thus be tolerated without compromising function. Preferably, such variant is capable of maintaining the activity of said above cited human cytochrome P450 and is capable of oxidizing small foreign organic molecules (xenobiotics), such as toxins or drugs

In a particular embodiment, said engineered cells of the present invention can advantageously combine an expression of CYP2B6 gene to confer hypersensitivity to cyclosphosphamide and/or isophosphamide and said and a further genetic engineering to confer specific resistance to another drug, such additional modification may be performed by a gene inactivation or by overexpression of a wild-type or a mutant form of a gene, said gene being involved in the metabolization of said second drug.

In another particular embodiment, said further genetic engineered of cells according to the present invention, in addition to the gene expression of CYP2B6, confers resistance to said second drug selected in the group consisting of alkylating agents (other than cyclophosphamide and isophosphamide), metabolic antagonists (e.g., purine nucleoside antimetabolite such as clofarabine, fludarabine or 2′-deoxyadenosine, 5-fluorouracil or derivatives thereof), antitumor antibiotics (e.g., mitomycin, adriamycin), plant-derived antitumor agents (e.g., vincristine, vindesine, Taxol), cisplatin, carboplatin, etoposide, TRIMETHOTRIXATE™ (TMTX), TEMOZOLOMIDE™, RALTRITREXED™, S-(4-Nitrobenzyl)-6-thioinosine (NBMPR),6-benzyguanidine (6-BG), bis-chloronitrosourea (BCNU) and CAMPTOTHECIN™, immunomodulating agents such as thalidomide (Thalomid®) Lenalidomide (Revlimid®) Pomalidomide (Pomalyst®), proteasome inhibitors such as Bortezomib (Velcade®), Carfilzomib (Kyprolis®), Histone deacetylase (HDAC) inhibitors such as Panobinostat (Farydak®), or a therapeutic derivative of any thereof.

In a more particular embodiment, said engineered cells of the present invention can advantageously combine an expression of CYP2B6 gene to confer hypersensitivity to cyclosphosphamide and/or isophosphamide and said further genetic engineering being a gene inactivation of a gene selected in the group of deoxycytidine kinase (dCk), hypoxanthine guanine phosphoribosyl transferase (HPRT), glucocorticoid receptor (GR) and CD52, conferring specific drug resistance to purine nucleoside analogues (PNAs)—such as clofarabine or fludarabine—, corticosteroids, alemtuzumab respectively.

Referring to the previous embodiment, said engineered cells of the present invention can advantageously combine an expression of CYP2B6 gene to confer hypersensitivity to cyclosphosphamide and/or isophosphamide and said further genetic engineering is inactivation of dCk gene conferring specific drug resistance to purine nucleoside analogues (PNAs)—such as clofarabine or fludarabine.

As exemplary embodiments, said engineered cells of the present invention can advantageously combine an expression of CYP2B6 gene to confer hypersensitivity to cyclosphosphamide and/or isophosphamide and said further genetic engineering being a gene expression of a mutated gene selected in the group consisting of dihydrofolate reductase (DHFR) inosine monophosphate dehydrogenase 2 (IMPDH2), calcineurin (PP2B) and methylguanine transferase (MGMT), conferring specific drug resistance to respectively anti-folate preferably methotrexate (MTX), to MPDH inhibitors such as mycophenolic acid (MPA) or its prodrug mycophenolate mofetil (MMF), to calcineurin inhibitor such as FK506 and/or Cs and to alkylating agents, such as nitrosoureas and temozolomide (TMZ).

The above mutated genes such as DHFR, IMPDH2, PP2B, MGMT can be obtained such as described in WO 2015075195.

As other exemplary embodiments, said engineered cells of the present invention can advantageously combine an expression of CYP2B6 gene to confer hypersensitivity to cyclosphosphamide and/or isophosphamide and said further genetic engineering being a gene expression of a wild type gene selected in the group consisting of MDR1, ble and mcrA, conferring specific drug resistance to respectively MDR1 resistance drugs such as 4-nitroquinoline-N-oxide, cerulenin, and brefeldin A, to bleomycin and to mitomycin C.

According to another embodiment, said engineered cells of the present invention can advantageously combine an expression of CYP2B6 gene to confer hypersensitivity to cyclosphosphamide and/or isophosphamide and said further genetic engineering is an expression of another gene which confers a supplemental hypersensitivity to another specific drug, preferably one gene selected in the group consisting of CYP2D6-2, CDA, CYP2C9, CYP3A4, CYP2D6-1, CYP2C19 and CYP1A2 conferring drug-specific hypersensitivity to cyclophosphamide and/or isophosphamide.

Said method can be used to produce engineered cells for treating cancer, infection or immune disease in a patient by unique or sequential administration thereof to a patient.

As a preferred embodiment, the invention provides the administration of an immune cell made hypersensitive to cyclosphosphamide and/or isophosphamide drug by expressing the CYP2B6 gene, said cell being further engineered to endow a chimeric antigen receptor (CAR) against said cancerous cell, infectious agent or dysfunctioning host immune cell.

Overexpression of CYP1A2 to Confer Hypersensitivity to Cyclosphosphamide and/or Isophosphamide

The immune cells according to the present invention, which CYP1A2 is expressed, are produced to be administered to the patient prior to their elimination by the cyclosphosphamide and/or isophosphamide drug in case of need (such as occurrence of an adverse event). Expression of CYP1A2 has been found by the inventors to confer sensitivity of cells derived from lymphoid progenitor cells, such as NK cells and T cells, to cyclosphosphamide and/or isophosphamide. Thus, to modulate or terminate the treatment, further administration of cyclosphosphamide and/or isophosphamide to which said cells have been made sensitive may be performed in order to deplete in vivo said cells.

According to one embodiment, the present invention relates to a method of producing human cell that may be depleted in-vivo as part of a cell therapy or immunotherapy treatment, said method comprising:

(a) Providing a human cell;

(b) Inducing hypersensitivity to cyclosphosphamide and/or isophosphamide into said cell by selectively expressing or overexpressing at least CYP1A2 transgene involved in the mechanism of action of said drug,

(c) Optionally assaying the hypersensitivity to said drug of said cell engineered in step b);

(d) Expanding said engineered cell obtained in step b).

In another specific embodiment, the gene encoding for the human cytochrome P450 1A2 (CYP1A2) which is used in the present invention to be expressed is the one of SEQ ID NO. 26 (GAAGCTCCACACCAGCCATTACAACCCTGCCAATCTCAAGCACCTGCCTCTACAGTTGGTACAGATGGCATTGT CCCAGTCTGTTCCCTTCTCGGCCACAGAGCTTCTCCTGGCCTCTGCCATCTTCTGCCTGGTATTCTGGGTGCTCA AGGGTTTGAGGCCTCGGGTCCCCAAAGGCCTGAAAAGTCCACCAGAGCCATGGGGCTGGCCCTTGCTCGGGC ATGTGCTGACCCTGGGGAAGAACCCGCACCTGGCACTGTCAAGGATGAGCCAGCGCTACGGGGACGTCCTGC AGATCCGCATTGGCTCCACGCCCGTGCTGGTGCTGAGCCGCCTGGACACCATCCGGCAGGCCCTGGTGCGGCA GGGCGACGATTTCAAGGGCCGGCCTGACCTCTACACCTCCACCCTCATCACTGATGGCCAGAGCTTGACCTTCA GCACAGACTCTGGACCGGTGTGGGCTGCCCGCCGGCGCCTGGCCCAGAATGCCCTCAACACCTTCTCCATCGC CTCTGACCCAGCTTCCTCATCCTCCTGCTACCTGGAGGAGCATGTGAGCAAGGAGGCTAAGGCCCTGATCAGC AGGTTGCAGGAGCTGATGGCAGGGCCTGGGCACTTCGACCCTTACAATCAGGTGGTGGTGTCAGTGGCCAAC GTCATTGGTGCCATGTGCTTCGGACAGCACTTCCCTGAGAGTAGCGATGAGATGCTCAGCCTCGTGAAGAACA CTCATGAGTTCGTGGAGACTGCCTCCTCCGGGAACCCCCTGGACTTCTTCCCCATCCTTCGCTACCTGCCTAACC CTGCCCTGCAGAGGTTCAAGGCCTTCAACCAGAGGTTCCTGTGGTTCCTGCAGAAAACAGTCCAGGAGCACTA TCAGGACTTTGACAAGAACAGTGTCCGGGACATCACGGGTGCCCTGTTCAAGCACAGCAAGAAGGGGCCTAG AGCCAGCGGCAACCTCATCCCACAGGAGAAGATTGTCAACCTTGTCAATGACATCTTTGGAGCAGGATTTGAC ACAGTCACCACAGCCATCTCCTGGAGCCTCATGTACCTTGTGACCAAGCCTGAGATACAGAGGAAGATCCAGA AGGAGCTGGACACTGTGATTGGCAGGGAGCGGCGGCCCCGGCTCTCTGACAGACCCCAGCTGCCCTACTTGG AGGCCTTCATCCTGGAGACCTTCCGACACTCCTCCTTCTTGCCCTTCACCATCCCCCACAGCACAACAAGGGACA CAACGCTGAATGGCTTCTACATCCCCAAGAAATGCTGTGTCTTCGTAAACCAGTGGCAGGTCAACCATGACCCA GAGCTGTGGGAGGACCCCTCTGAGTTCCGGCCTGAGCGGTTCCTCACCGCCGATGGCACTGCCATTAACAAGC CCTTGAGTGAGAAGATGATGCTGTTTGGCATGGGCAAGCGCCGGTGTATCGGGGAAGTCCTGGCCAAGTGGG AGATCTTCCTCTTCCTGGCCATCCTGCTACAGCAACTGGAGTTCAGCGTGCCGCCGGGCGTGAAAGTCGACCTG ACCCCCATCTACGGGCTGACCATGAAGCACGCCCGCTGTGAACATGTCCAGGCGCGGCTGCGCTTCTCCATCA ATTGAAGAAGACACCACCATTCTGAGGCCAGGGAGCGAGTGGGGGCCAGCCACGGGGACTCAGCCCTTGTTT CTCTTCCTTTCTTTTTTTAAAAAATAGCAGCTTTAGCCAAGTGCAGGGCCTGTAATCCCAGCATTTTAGGAGGCC AAGGTTGGAGGATCATTTGAGCCCAGGAATTGGAAAGCAGCCTGGCCAACATAGTGGGACCCTGTCTCTACA AAAAAAAAATTTGCCAAGAGCCTGAGTGACAGAGCAAGACCCCATCTCAAAAAAAAAAACAAACAAACAAAA AAAAAACCATATATATACATATATATATAGCAGCTTTATGGAGATATAATTCTTATGCCATATAATTCACCTTCTT TTTTTTTTTTTGTCTGAGACAGAATCTCAGTCTGTCACCCAGGTTGGAGTGCAGTGGCGTGATCTCAGCTCACTG CAACCTCCACCTCGCAGGTTCAAGCAATCCTCCCACTTCAGCCTCCCAAGCACCTGGGATTACAAGCATGAGTC ACTACGCCTGGCTGATTTTTGTAGTTTTAGTGGAGATGGGGTTTCACCATGTTGGCCAGGCTTGTCTCGAACTC CTGACCCCAAGTTATCCACCTGCCTTGGCTTCCCAAAGTCCTGGGATTACAGGTGTGAGCCACCACATCCAGCC TAACTTACATTCTTAAAGTGTCGAATGACTTCTAGTGTAGAATTGTGCAACCATCACCAGAATTAATTTTATTAT TCTTATTATTTTTGAGACAGAGTCTTACTCTGTTGCCAGGCTGGAGTGCAGTGGCGCGATCTCAGCTCACTACA ACCTCCGCCTCCCATGTTCAAGCGATTCTCCTGCCTCAGCCTCCCGAGTAGCTGGGACTATAGGCATGCGCCAC CATGGCCAGCTAATTTTTGTATTTTTAGTAGAGACGAGGTTTCACTGTGTTGGCCAGGATGGTCTCCATCTCTTG ACCTCGTGATCCACCCGCCTCAGCCTCCCAAAGTGCTGGGATTAACAGGTATGAACCACCGCGCCCAGCCTTTT TGTTTTTTTTTTTTTTGAGACAGAGTCTTCCTCTGTCTCCTAAGCTGGAGTGCAGTGGCATCATCTCAGCTCACTG CAACCTCTGCCTCCCAGGTTCAAGTGCTTCTCCAGCCTCAGCCTCCCAAGTAGCTGAGACTACAGGCACACACC ACCACGCCTGGCTAATTTTTGTATTTTTAGTAGAGACGGGTTTCACCATGTTGGCTAGACTAGTCTCAAACTCCT GACCTCAAGTGATCTGCCCGCCTCGACCTCTCTCAAAGTGCTGGCATTACAGGTGTGAGCCACGGTGCCCGGC CCACAATTAATTTTAGAACATTTTCATCACCCCTAAAAGAAACCCTGCACCCATTAGCAGTCCCTCCACATTTCCC CCTAGCCTGCCTCCCCTGCCTCACCAGCCCTGGCAACTGCTAATCTACTTTCTGTGTCTATGGATTTGCCTTCTCT AAACATTTCATATAAATGGAATTACACAATG) RefSeq n^{o} NP_000752, or a variant thereof comprising a nucleotide sequence that has at least 60%, such as at least 80%, at least 85%, at least 90% or at least 95%, sequence identity with the amino acid sequence set forth in SEQ ID NO: 26 over the entire length of SEQ ID NO: 26. However, it is understood that due to the degeneration of the genetic code any other suitable nucleotide sequence coding for the amino acid sequence set forth in SEQ ID NO: 26 is also encompassed by the present disclosure.

Accordingly, in certain embodiment of the present invention, the human cytochrome P450 1A2 to be expressed comprises a polypeptide of SEQ ID NO: 7 (MALSQSVPFSATELLLASAIFCLVFWVLKGLRPRVPKGLKSPPEPWGWPLLGHVLTLGKNPHLALSRMSQRYGDVL QIRIGSTPVLVLSRLDTIRQALVRQGDDFKGRPDLYTSTLITDGQSLTFSTDSGPVWAARRRLAQNALNTFSIASDPAS SSSCYLEEHVSKEAKALISRLQELMAGPGHFDPYNQVVVSVANVIGAMCFGQHFPESSDEMLSLVKNTHEFVETAS SGNPLDFFPILRYLPNPALQRFKAFNQRFLWFLQKTVQEHYQDFDKNSVRDITGALFKHSKKGPRASGNLIPQEKIV NLVNDIFGAGFDTVTTAISWSLMYLVTKPEIQRKIQKELDTVIGRERRPRLSDRPQLPYLEAFILETFRHSSFLPFTIPHS TTRDTTLNGFYIPKKCCVFVNQWQVNHDPELWEDPSEFRPERFLTADGTAINKPLSEKMMLFGMGKRRCIGEVLA KWEIFLFLAILLQQLEFSVPPGVKVDLTPIYGLTMKHARCEHVQARLRFSIN), corresponding to P05177 (Ref Uniprot), or a variant thereof comprising an amino acid sequence that has at least 60%, such as at least 80%, at least 85%, at least 90% or at least 95%, sequence identity with the amino acid sequence set forth in SEQ ID NO: 7 over the entire length of SEQ ID NO: 7. The variant may comprise an amino acid sequence which has one or more, such as two, three, four, five or six amino acid substitutions compared to SEQ ID NO: 7.

Preferably, such amino acid substitution is a conservative substitution which means that one amino acid is replaced by another one that is similar in size and chemical properties. Such conservative amino acid substitution may thus have minor effects on the peptide structure and can thus be tolerated without compromising function. Preferably, such variant is capable of maintaining the activity of human cytochrome P450 1A2 and is capable of oxidizing organic molecules such as drugs.

In a particular embodiment, said engineered cells of the present invention can advantageously combine an expression of CYP1A2 gene to confer hypersensitivity to cyclosphosphamide and/or isophosphamide and said and a further genetic engineering to confer specific resistance to another drug, such additional modification may be performed by a gene inactivation or by overexpression of a wild-type or a mutant form of a gene, said gene being involved in the metabolization of said second drug.

In another particular embodiment, said further genetic engineered of cells according to the present invention, in addition to the gene expression of CYP1A2, confers resistance to said second drug selected in the group consisting of alkylating agents (other than cyclophosphamide and isophosphamide), metabolic antagonists (e.g., purine nucleoside antimetabolite such as clofarabine, fludarabine or 2′-deoxyadenosine, 5-fluorouracil or derivatives thereof), antitumor antibiotics (e.g., mitomycin, adriamycin), plant-derived antitumor agents (e.g., vincristine, vindesine, Taxol), cisplatin, carboplatin, etoposide, TRIMETHOTRIXATE™ (TMTX), TEMOZOLOMIDE™, RALTRITREXED™, S-(4-Nitrobenzyl)-6-thioinosine (NBMPR),6-benzyguanidine (6-BG), bis-chloronitrosourea (BCNU) and CAMPTOTHECIN™, immunomodulating agents such as thalidomide (Thalomid®) Lenalidomide (Revlimid®) Pomalidomide (Pomalyst®), proteasome inhibitors such as Bortezomib (Velcade®), Carfilzomib (Kyprolis®), Histone deacetylase (HDAC) inhibitors such as Panobinostat (Farydak®), or a therapeutic derivative of any thereof.

In a more particular embodiment, said engineered cells of the present invention can advantageously combine an expression of CYP1A2 gene to confer hypersensitivity to cyclosphosphamide and/or isophosphamide and said further genetic engineering being a gene inactivation of a gene selected in the group of deoxycytidine kinase (dCk), hypoxanthine guanine phosphoribosyl transferase (HPRT), glucocorticoid receptor (GR) and CD52, conferring specific drug resistance to purine nucleoside analogues (PNAs)—such as clofarabine or fludarabine—, corticosteroids, alemtuzumab respectively.

Referring to the previous embodiment, said engineered cells of the present invention can advantageously combine an expression of CYP1A2 gene to confer hypersensitivity to cyclosphosphamide and/or isophosphamide and said further genetic engineering is inactivation of dCk gene conferring specific drug resistance to purine nucleoside analogues (PNAs)—such as clofarabine or fludarabine.

As exemplary embodiments, said engineered cells of the present invention can advantageously combine an expression of CYP1A2 gene to confer hypersensitivity to cyclosphosphamide and/or isophosphamide and said further genetic engineering being a gene expression of a mutated gene selected in the group consisting of dihydrofolate reductase (DHFR) inosine monophosphate dehydrogenase 2 (IMPDH2), calcineurin (PP2B) and methylguanine transferase (MGMT), conferring specific drug resistance to respectively anti-folate preferably methotrexate (MTX), to MPDH inhibitors such as mycophenolic acid (MPA) or its prodrug mycophenolate mofetil (MMF), to calcineurin inhibitor such as FK506 and/or Cs and to alkylating agents, such as nitrosoureas and temozolomide (TMZ).

The above mutated genes such as DHFR, IMPDH2, PP2B, MGMT can be obtained such as described in WO 2015075195.

As other exemplary embodiments, said engineered cells of the present invention can advantageously combine an expression of CYP1A2 gene to confer hypersensitivity to cyclosphosphamide and/or isophosphamide and said further genetic engineering being a gene expression of a wild type gene selected in the group consisting of MDR1, ble and mcrA, conferring specific drug resistance to respectively MDR1 resistance drugs such as 4-nitroquinoline-N-oxide, cerulenin, and brefeldin A, to bleomycin and to mitomycin C.

According to another embodiment, said engineered cells of the present invention can advantageously combine an expression of CYP1A2 gene to confer hypersensitivity to cyclosphosphamide and/or isophosphamide and said further genetic engineering is an expression of another gene which confers a supplemental hypersensitivity to another specific drug, preferably one gene selected in the group consisting of CYP2D6-2, CDA, CYP2C9, CYP3A4, CYP2D6-1, CYP2C19 and CYP2B6 conferring drug-specific hypersensitivity to cyclophosphamide and/or isophosphamide.

Said method can be used to produce engineered cells for treating cancer, infection or immune disease in a patient by unique or sequential administration thereof to a patient.

As a preferred embodiment, the invention provides the administration of an immune cell made hypersensitive to cyclosphosphamide and/or isophosphamide drug by expressing the CYP1A2 gene, said cell being further engineered to endow a chimeric antigen receptor (CAR) against said cancerous cell, infectious agent or dysfunctioning host immune cell.

Delivery Method

The different methods described below involve expressing a protein of interest such as prodrug hypersensitivity related gene, prodrug resistance related gene, rare-cutting endonuclease, Chimeric Antigen Receptor (CAR), immune checkpoint or suicide gene into a cell.

In accordance with the present invention, the nucleic acid molecules detailed herein may be introduced in the human cell, preferably immune cell (i.e T-cell) by any suitable methods known in the art. Suitable, non-limiting methods for introducing a nucleic acid molecule into a human cell, preferably immune cell according include stable transformation methods, wherein the nucleic acid molecule is integrated into the genome of the cell, transient transformation methods wherein the nucleic acid molecule is not integrated into the genome of the cell and virus mediated methods. Said nucleic acid molecule may be introduced into a cell by, for example, a recombinant viral vector (e.g., retroviruses, adenoviruses), liposome and the like. Transient transformation methods include, for example, microinjection, electroporation or particle bombardment. In certain embodiments, the nucleic acid molecule is a vector, such as a viral vector or plasmid. Suitably, said vector is an expression vector enabling the expression of the respective polypeptide(s) or protein(s) detailed herein by the immune cell.

A nucleic acid molecule introduced into the human cell, preferably immune cell may be DNA or RNA. In certain embodiments, a nucleic acid molecule introduced into the human cell, preferably immune cell is DNA. In certain embodiments, a nucleic acid molecule introduced into said cell is RNA, and in particular an mRNA encoding a polypeptide or protein detailed herein, which mRNA is introduced directly into the immune cell, for example by electroporation. A suitable electroporation technique is described, for example, in International Publication WO2013/176915 (in particular the section titled “Electroporation” bridging pages 29 to 30).

In one embodiment, said transgene conferring specific drug hypersensitivity such as disclosed in the method of the present invention is transfected into an human cell, preferably immune cell by a delivery vector.

By “delivery vector” is intended any delivery vector which can be used in the present invention to put into cell contact (i.e “contacting”) or deliver inside cells or subcellular compartments (i.e “introducing”) agents/chemicals and molecules (proteins or nucleic acids) needed in the present invention. It includes, but is not limited to liposomal delivery vectors, viral delivery vectors, prodrug delivery vectors, chemical carriers, polymeric carriers, lipoplexes, polyplexes, dendrimers, microbubbles (ultrasound contrast agents), nanoparticles, emulsions or other appropriate transfer vectors.

In a preferred embodiment, the delivery vector for expressing transgene into a human cell, preferably immune cell is a viral vector and more preferably a lentivirus vector.

The terms “vector” refer to a nucleic acid molecule capable of transporting another nucleic acid to which it has been linked. A “vector” in the present invention includes, but is not limited to, a viral vector, a plasmid, a RNA vector or a linear or circular DNA or RNA molecule which may consist of a chromosomal, non chromosomal, semi-synthetic or synthetic nucleic acids. Preferred vectors are those capable of expression of nucleic acids to which they are linked (expression vectors). Large numbers of suitable vectors are known to those of skill in the art and commercially available

Said polynucleotides may be included in vectors, more particularly plasmids or virus, in view of being expressed in cells. Said plasmid vector can comprise a selection marker which provides for identification and/or selection of cells which received said vector. Different transgenes can be included in one vector. Said vector can comprise a nucleic acid sequence encoding ribosomal skip sequence such as a sequence encoding a 2A peptide. 2A peptides, which were identified in the Aphthovirus subgroup of picornaviruses, causes a ribosomal “skip” from one codon to the next without the formation of a peptide bond between the two amino acids encoded by the codons (see Donnelly et al., J. of General Virology 82: 1013-1025 (2001); Donnelly et al., J. of Gen. Virology 78: 13-21 (1997); Doronina et al., Mol. And. Cell. Biology 28(13): 4227-4239 (2008); Atkins et al., RNA 13: 803-810 (2007)). By “codon” is meant three nucleotides on an mRNA (or on the sense strand of a DNA molecule) that are translated by a ribosome into one amino acid residue. Thus, two polypeptides can be synthesized from a single, contiguous open reading frame within an mRNA when the polypeptides are separated by a 2A oligopeptide sequence that is in frame. Such ribosomal skip mechanisms are well known in the art and are known to be used by several vectors for the expression of several proteins encoded by a single messenger RNA.

Viral vectors include retrovirus, adenovirus, parvovirus (e. g. adenoassociated viruses), coronavirus, negative strand RNA viruses such as orthomyxovirus (e. g., influenza virus), rhabdovirus (e. g., rabies and vesicular stomatitis virus), paramyxovirus (e. g. measles and Sendai), positive strand RNA viruses such as picornavirus and alphavirus, and double-stranded DNA viruses including adenovirus, herpesvirus (e. g., Herpes Simplex virus types 1 and 2, Epstein-Barr virus, cytomega-lovirus), and poxvirus (e. g. vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).

By “lentiviral vector” is meant HIV-Based lentiviral vectors that are very promising for gene delivery because of their relatively large packaging capacity, reduced immunogenicity and their ability to stably transduce with high efficiency a large range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration in the DNA of infected cells. By “integrative lentiviral vectors (or LV)”, is meant such vectors as non limiting example, that are able to integrate the genome of a target cell. At the opposite by “non-integrative lentiviral vectors (or NILV)” is meant efficient gene delivery vectors that do not integrate the genome of a target cell through the action of the virus integrase. Preferably, the lentiviral vectors are integrative ones and those which allow a high frequency of integration outside the coding regions of the genome in order to minimize the occurrence of potential side effects. These LT vectors comprise preferably a promotor which expression is constitutive, such as a SFFV promotor.

As another embodiment of the invention, polynucleotides encoding polypeptides according to the present invention can be mRNA which is introduced directly into the cells, for example by electroporation. The inventors determined the optimal condition for mRNA electroporation in T-cell. The inventor used the cytoPulse technology which allows, by the use of pulsed electric fields, to transiently permeabilize living cells for delivery of material into the cells. The technology, based on the use of PulseAgile (BTX Havard Apparatus, 84 October Hill Road, Holliston, Mass. 01746, USA) electroporation waveforms grants the precise control of pulse duration, intensity as well as the interval between pulses (U.S. Pat. No. 6,010,613 and International PCT application WO2004083379). All these parameters can be modified in order to reach the best conditions for high transfection efficiency with minimal mortality. Basically, the first high electric field pulses allow pore formation, while subsequent lower electric field pulses allow exporting the polynucleotide into the cell.

Gene Inhibition/Gene Inactivation by Rare Cutting Endonuclease

By “inhibiting the expression of at least one gene”, it is meant that the gene of interest is not expressed in a functional protein form or insufficiently to have a physiologic effect. This inhibition can be obtained by gene silencing (ex, RNAi, siRNA) or by gene edition, in preferably by knock-out mechanism, using in particular rare cutting and site-specific endonuclease such as meganuclease, TALE-nuclease or CRISPR-Cas9. This preference is based on the nature of the response by siRNA which is transient; the transduction of siRNA into cells leading to only a transient knockdown of the gene of interest. Moreover, gene expression is dependent upon siRNA concentration.

Moreover, “By inactivating a gene”, it is intended that the gene of interest is not expressed in a functional protein form. In particular embodiment, the genetic modification of the method relies on the expression, in provided cells to engineer, of one rare-cutting endonuclease such that said rare-cutting endonuclease specifically catalyzes cleavage in one targeted gene thereby inactivating said targeted gene.

In a particular embodiment, said rare-cutting endonuclease is used to inactivate at least one gene selected from those which confer an additional drug-specific hypersensitivity, drug-specific resistance, TCR gene and like which confer allogeneicity, immune checkpoint, suicide gene as presented before.

In a more particular embodiment, said drug resistance can be conferred to the T-cell by the inactivation of a drug sensitizing gene, therefore conferring resistance to its specific corresponding drug.

In a preferred embodiment, the metabolism drug related gene is inactivated by the use of a rare cutting specific endonuclease selected in a group consisting of TALE-nucleases, Zing Finger nucleases, Cas9, Cpf1, Argonaute, homing endonucleases, or meganucleases.

In a preferred embodiment, said rare-cutting endonuclease is a TALE-nuclease or Cas 9/CRISPR. By TALE-nuclease is intended a fusion protein consisting of a DNA-binding domain derived from a Transcription Activator Like Effector (TALE) and one nuclease catalytic domain to cleave a nucleic acid target sequence (Boch, Scholze et al. 2009; Moscou and Bogdanove 2009; Christian, Cermak et al. 2010; Cermak, Doyle et al. 2011; Geissler, Scholze et al. 2011; Huang, Xiao et al. 2011; Li, Huang et al. 2011; Mahfouz, Li et al. 2011; Miller, Tan et al. 2011; Morbitzer, Romer et al. 2011; Mussolino, Morbitzer et al. 2011; Sander, Cade et al. 2011; Tesson, Usal et al. 2011; Weber, Gruetzner et al. 2011; Zhang, Cong et al. 2011; Deng, Yan et al. 2012; Li, Piatek et al. 2012; Mahfouz, Li et al. 2012; Mak, Bradley et al. 2012). In the present invention new TALE-nucleases have been designed for precisely targeting relevant genes for adoptive immunotherapy strategies.

In particular embodiments, the genetic modification of the method relies on the expression, in provided cells to engineer, of one rare-cutting endonuclease such that said rare-cutting endonuclease specifically catalyzes cleavage in one targeted gene thereby inactivating said targeted gene. The nucleic acid strand breaks caused by the rare-cutting endonuclease are commonly repaired through the distinct mechanisms of homologous recombination or non-homologous end joining (NHEJ). However, NHEJ is an imperfect repair process that often results in changes to the DNA sequence at the site of the cleavage. Mechanisms involve rejoining of what remains of the two DNA ends through direct re-ligation (Critchlow and Jackson 1998) or via the so-called microhomology-mediated end joining (Betts, Brenchley et al. 2003; Ma, Kim et al. 2003). Repair via non-homologous end joining (NHEJ) often results in small insertions or deletions and can be used for the creation of specific gene knockouts. Said modification may be a substitution, deletion, or addition of at least one nucleotide. Cells in which a cleavage-induced mutagenesis event, i.e. a mutagenesis event consecutive to an NHEJ event, has occurred can be identified and/or selected by well-known method in the art.

The gene inactivation by KO using TALE nuclease may be performed such as described in the protocols provided in the section “general methods” within the present application.

In another embodiment, additional catalytic domain can be further introduced into the cell with said rare-cutting endonuclease to increase mutagenesis in order to enhance their capacity to inactivate targeted genes. In particular, said additional catalytic domain is a DNA end processing enzyme. Non limiting examples of DNA end-processing enzymes include 5-3′ exonucleases, 3-5′ exonucleases, 5-3′ alkaline exonucleases, 5′ flap endonucleases, helicases, phosphatase, hydrolases and template-independent DNA polymerases. Non limiting examples of such catalytic domain comprise of a protein domain or catalytically active derivate of the protein domain selected from the group consisting of hExol (EXO1_HUMAN), Yeast Exol (EXO1_YEAST), E. coli Exol, Human TREX2, Mouse TREX1, Human TREX1, Bovine TREX1, Rat TREX1, TdT (terminal deoxynucleotidyl transferase) Human DNA2, Yeast DNA2 (DNA2_YEAST). In a preferred embodiment, said additional catalytic domain has a 3′-5′-exonuclease activity, and in a more preferred embodiment, said additional catalytic domain is TREX, more preferably TREX2 catalytic domain (WO2012/058458). In another preferred embodiment, said catalytic domain is encoded by a single chain TREX2 polypeptide. Said additional catalytic domain may be fused to a nuclease fusion protein or chimeric protein according to the invention optionally by a peptide linker.

Endonucleolytic breaks are known to stimulate the rate of homologous recombination. Thus, in another embodiment, the genetic modification step of the method further comprises a step of introduction into cells of an exogenous nucleic acid comprising at least a sequence homologous to a portion of the target nucleic acid sequence, such that homologous recombination occurs between the target nucleic acid sequence and the exogenous nucleic acid.

According to one embodiment, inhibition of the expression of such gene implicated in the drug metabolization conferring resistance to said drug, is obtained by introducing into said cell at least one rare-cutting endonucleases targeting said gene. Said rare-cutting endonuclease may introduce a mutation inactivating or reducing the expression of said gene.

In a particular embodiment, the step of inactivating at least a gene encoding an enzyme implicated in the drug metabolization conferring resistance to said drug comprises introducing into the cell a rare-cutting endonuclease able to specifically disrupt at least one gene encoding said enzyme.

In particular embodiment, the genetic modification of the method relies on the expression, in provided cells to engineer, of one rare-cutting endonuclease such that said rare-cutting endonuclease specifically catalyzes cleavage in one targeted gene thereby inactivating said targeted gene implicated in the drug metabolization conferring resistance to said drug.

In particular embodiments, said exogenous nucleic acid comprises first and second portions which are homologous to region 5′ and 3′ of the target nucleic acid sequence, respectively. Said exogenous nucleic acid in these embodiments also comprises a third portion positioned between the first and the second portion which comprises no homology with the regions 5′ and 3′ of the target nucleic acid sequence. Following cleavage of the target nucleic acid sequence, a homologous recombination event is stimulated between the target nucleic acid sequence and the exogenous nucleic acid. Preferably, homologous sequences of at least 50 bp, preferably more than 100 bp and more preferably more than 200 bp are used within said donor matrix. In a particular embodiment, the homologous sequence can be from 200 bp to 6000 bp, more preferably from 1000 bp to 2000 bp. Indeed, shared nucleic acid homologies are located in regions flanking upstream and downstream the site of the break and the nucleic acid sequence to be introduced should be located between the two arms.

Chemotherapeutic agent used as drug in case of further engineered immune cells to make them resistant to a specific drug refers herein to a compound or a derivative thereof that can interact with a cancer cell, thereby reducing the proliferative status of the cell and/or killing the cancer cell, while preserving the engineered immune cells. Examples of chemotherapeutic agents include, but are not limited to, alkylating agents (excepted cyclophosphamide and cyclosphosphamide when at least one of these are used as depleting drug), metabolic antagonists (e.g., methotrexate (MTX), 5-fluorouracil or derivatives thereof), antitumor antibiotics (e.g., mitomycin, adriamycin), plant-derived antitumor agents (e.g., vincristine, vindesine, Taxol), cisplatin, carboplatin, etoposide, and the like. Such agents may further include, but are not limited to, the anti-cancer agents TRIMETHOTRIXATE™ (TMTX), TEMOZOLOMIDE™, RALTRITREXED™, S-(4-Nitrobenzyl)-6-thioinosine (NBMPR),6-benzyguanidine (6-BG), bis-chloronitrosourea (BCNU) and CAMPTOTHECIN™, or a therapeutic derivative of any thereof.

It is understood that this additional step of engineering can be performed after the b) step i.e. after the overexpression of human cells, preferably immune cells to make them hypersensitive to a specific drug, but it can be performed before said overexpression step.

Consequently, according to one embodiment, the method of producing human cell, preferably immune cell that may be depleted in-vivo as part of an immunotherapy treatment, said method comprising the following sequential steps of:

(a) Providing an immune cell;

(b) Inducing prodrug hypersensitivity into said cell by selectively expressing or overexpressing at least one transgene involved in the specific conversion of prodrug to drug which is toxic to said immune cell,

(c) Inducing drug resistance into said cell by selectively inactivating at least one gene involved in the specific metabolization, elimination of detoxification of its other specific drug(s); said drug(s) being different to that of (b);

(d) Optionally assaying the hypersensitivity to said prodrug and/or resistance to said specific drug(s) of the cell engineered in step (c);

(e) Expanding the engineered immune cells obtained in step b).

According to an alternative embodiment, the method of producing human cell, preferably immune cell that may be depleted in-vivo as part of an immunotherapy treatment, said method comprising the following sequential steps of:

(a) Providing an immune cell;

(b) Inducing drug resistance into said cell by selectively inactivating at least one gene involved in the specific metabolization, elimination of detoxification of its specific drug(s);

(c) Inducing prodrug hypersensitivity into said cell by selectively expressing or overexpressing at least one transgene involved in the specific conversion of prodrug to a specific drug which is toxic to said immune cell, said drug being different to that in (b);

(d) Optionally assaying the hypersensitivity to said prodrug and/or resistance to said other specific drug(s) of the cell engineered in step (c);

(e) Expanding the engineered immune cells obtained in step b).

In a particular embodiment, the dCK inactivation in T cells is combined with an inactivation of TRAC genes rendering these double knock out (KO) T cells both resistant to drug such as clofarabine and allogeneic. This double features is particularly useful for a therapeutic goal, allowing “off-the-shelf” allogeneic cells for immunotherapy in conjunction with chemotherapy to treat patients with cancer. Such aspect is disclosed in WO2013176915.

Expression of Chimeric Antigen Receptor (CAR)

According to one preferred embodiment, said drug specific hypersensitive engineered immune cells obtained according to the method of the present invention, are further engineered to express a Chimeric Antigen Receptor (CAR).

By “chimeric antigen receptor (CAR)”, it is meant a chimeric receptor which comprises an extracellular ligand-binding domain, a transmembrane domain and a signaling transducing domain. Chimeric Antigen Receptors (CAR) are able to redirect immune cell specificity and reactivity toward a selected target exploiting the ligand-binding domain properties. Said Chimeric Antigen Receptor combines a binding domain against a component present on the target cell, for example an antibody-based specificity for a desired antigen (e.g., tumor antigen) with a T-cell receptor-activating intracellular domain to generate a chimeric protein that exhibits a specific anti-target cellular immune activity. Generally, CAR consists of an extracellular single chain antibody (scFv) fused to the intracellular signaling domain of the T-cell antigen receptor complex zeta chain (scFv:ζ) and have the ability, when expressed in T-cells, to redirect antigen recognition based on the monoclonal antibody's specificity.

Thus, in another particular embodiment, the method further comprises a step of introducing into said lymphocytes a Chimeric Antigen Receptor.

The introduction of chimeric antigen receptor (CAR) into immune cells, such as T cells, and the characterization said CAR-expressing cells may be performed according to the protocols provided in the section “general methods” within the present application.

Specific chimeric antigen receptors according to the invention can have different architectures, as they can be expressed, for instance, under a single-chain chimeric protein (scCAR) or under the form of several polypeptides (multi-chain CAR or mcCAR) including at least one such chimeric protein.

According to one embodiment, said chimeric antigen receptor which is expressed by immune cell is a CD123+, CD19+, CS1+, CD38+, ROR1+, CLL1+, hsp70+, CD22+, EGFRvIII+, BCMA+, CD33+, FLT3+, CD70+, WT1+, MUC16+, PRAME+, TSPAN10+, ROR1+, GD3+, CT83+, mesothelin+.

The term “extracellular ligand-binding domain” as used herein is defined as an oligo- or polypeptide that is capable of binding a ligand. Preferably, the domain will be capable of interacting with a cell surface molecule. For example, the extracellular ligand-binding domain may be chosen to recognize a ligand that acts as a cell surface marker on target cells associated with a particular disease state.

In a preferred embodiment, said extracellular ligand-binding domain comprises a single chain antibody fragment (scFv) comprising the light (V_{L}) and the heavy (V_{H}) variable fragment of a target antigen specific monoclonal antibody joined by a flexible linker.

The signal transducing domain or intracellular signaling domain of the CAR according to the present invention is responsible for intracellular signaling following the binding of extracellular ligand binding domain to the target resulting in the activation of the immune cell and immune response. Preferred examples of signal transducing domain for use in a CAR can be the cytoplasmic sequences of the T-cell receptor and co-receptors that act in concert to initiate signal transduction following antigen receptor engagement. Signal transduction domain comprises two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs of ITAMs. In particular embodiment the signal transduction domain of the CAR of the present invention comprises a co-stimulatory signal molecule. A co-stimulatory molecule is a cell surface molecule other than an antigen receptor or their ligands that is required for an efficient immune response. Co-stimulatory molecules include, but are not limited to an MHC class I molecule, BTLA and Toll ligand receptor. Examples of costimulatory molecules include CD27, CD28, CD8, 4-1BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83 and the like.

The CAR according to the present invention is expressed on the surface membrane of the cell. Thus, the CAR can comprise a transmembrane domain. The distinguishing features of appropriate transmembrane domains comprise the ability to be expressed at the surface of a cell, preferably in the present invention an immune cell, in particular lymphocyte cells or Natural killer (NK) cells, and to interact together for directing cellular response of immune cell against a predefined target cell. The transmembrane domain can further comprise a stalk region_between said extracellular ligand-binding domain and said transmembrane domain. The term “stalk region” used herein generally means any oligo- or polypeptide that functions to link the transmembrane domain to the extracellular ligand-binding domain. In particular, stalk region are used to provide more flexibility and accessibility for the extracellular ligand-binding domain. A stalk region may comprise up to 300 amino acids, preferably 10 to 100 amino acids and most preferably 25 to 50 amino acids. Stalk region may be derived from all or part of naturally occurring molecules, such as from all or part of the extracellular region of CD8, CD4 or CD28, or from all or part of an antibody constant region. Alternatively the stalk region may be a synthetic sequence that corresponds to a naturally occurring stalk sequence, or may be an entirely synthetic stalk sequence.

Downregulation or mutation of target antigens is commonly observed in cancer cells, creating antigen-loss escape variants. Thus, to offset tumor escape and render immune cells more specific to target, the CD19 specific CAR can comprise another extracellular ligand-binding domains, to simultaneously bind different elements in target thereby augmenting immune cell activation and function. Examples of CD19 specific CAR are ScFv FMC63 (Kochenderfer J N, Wilson W H, Janik J E, et al. Eradication of B-lineage cells and regression of lymphoma in a patient treated with autologous T cells genetically engineered to recognize CD19. Blood 2010; 116(20):4099-410) or ScFv 4G7 CAR (described in the application filed under the number PCT/EP2014/059662). In one embodiment, the extracellular ligand-binding domains can be placed in tandem on the same transmembrane polypeptide, and optionally can be separated by a linker. In another embodiment, said different extracellular ligand-binding domains can be placed on different transmembrane polypeptides composing the CAR. In another embodiment, the present invention relates to a population of CARs comprising each one different extracellular ligand binding domains. In a particular, the present invention relates to a method of engineering immune cells comprising providing an immune cell and expressing at the surface of said cell a population of CAR each one comprising different extracellular ligand binding domains. In another particular embodiment, the present invention relates to a method of engineering an immune cell comprising providing an immune cell and introducing into said cell polynucleotides encoding polypeptides composing a population of CAR each one comprising different extracellular ligand binding domains. By population of CARs, it is meant at least two, three, four, five, six or more CARs each one comprising different extracellular ligand binding domains. The different extracellular ligand binding domains according to the present invention can preferably simultaneously bind different elements in target thereby augmenting immune cell activation and function. The present invention also relates to an isolated immune cell which comprises a population of CARs each one comprising different extracellular ligand binding domains.

In a preferred embodiment, said CAR which are expressed in the drug specific hypersensitive engineered immune cell such as described earlier is chosen in the group consisting of anti-CD123 CAR, anti-CS1 CAR, anti-CD38 CAR, anti-CLL1 CAR, anti-Hsp70 CAR, anti-CD22, anti-EGFRvIII, anti-BCMA CAR, anti-CD33 CAR, anti-FLT3 CAR, anti-CD70 CAR, anti-WT1 CAR, anti-MUC16 CAR, anti-PRAME CAR, anti-TSPAN10 CAR, anti-ROR1 CAR, anti-GD3 CAR, anti-CT83 CAR and anti-mesothelin CAR.

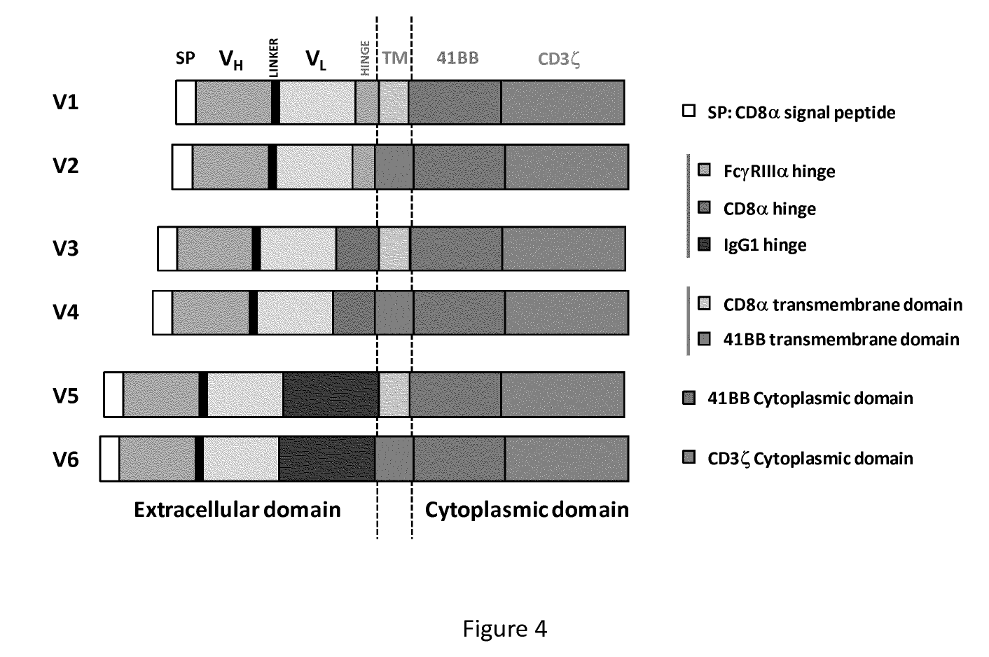

In a preferred embodiment, said above CAR is single-chain CAR chosen in the group consisting of anti-CD123 single-chain CAR, anti-CS1 single-chain CAR, anti-CD38 single-chain CAR, anti-CLL1 single-chain CAR, anti-Hsp70 single-chain CAR, anti-single-chain CD22, anti-EGFRvIII single-chain CAR, anti-BCMA single-chain CAR, anti-CD33 single-chain CAR, anti-FLT3 single-chain CAR, anti-CD70 single-chain CAR, anti-WT1 single-chain CAR, anti-MUC16 single-chain CAR, anti-PRAME single-chain CAR, anti-TSPAN10 single-chain CAR, anti-ROR1 single-chain CAR, anti-GD3 single-chain CAR, anti-CT83 single-chain CAR and mesothelin single-chain CAR;
said CAR being expressed in an immune cell initially engineered to be made hypersensitive to a specific prodrug has one of the polypeptide structure selected from V1, V3 or V5, as illustrated in FIG. 4;
said structure comprising:
   an extra cellular ligand binding-domain comprising VH and VL from a monoclonal antibody selected in the group consisting of anti-CD123 mAb, anti-CS1 mAb, anti-CD38 mAb, anti-CLL1 mAb, anti-Hsp70 mAb, anti-EGFRvIII mAb, anti-BCMA mAb, anti-CD33 mAb, anti-FLT3 mAb, anti-CD70 mAb, anti-WT1 mAb, anti-MUC16 mAb, anti-PRAME mAb, anti-TSPAN10 mAb, anti-ROR1 mAb, anti-GD3 mAb, anti-CT83 mAb and anti-mesothelin mAb respectively;
   a hinge chosen in the group consisting of CD8alpha, FcERIIIgamma and IgG1;
   a CD8α transmembrane domain;
   a cytoplasmic domain including a CD3 zeta signaling domain and;
   a 4-1BB co-stimulatory domain.

As examples, VH and VL may be those described in the applications WO2015140268 for anti-CD123, WO2015121454 for anti-CS1 and anti-CD38.

All the other components chosen in the architecture of the CAR including transmembrane domain (i.e CD8αTM), co-stimulatory domain (ie. 4-1BB), hinge (CD8alpha, FcERIIIgamma, IgG1), cytoplasmic signaling domain (ITAM CD3zeta) may be those already described in the above WO2015140268 and WO2015121454 applications.

In an embodiment, said above CAR is multi-chain CAR chosen in the group consisting of anti-CD123 multi-chain CAR, anti-CS1 multi-chain CAR, anti-CD38 multi-chain CAR, anti-CLL1 multi-chain CAR, anti-Hsp70 multi-chain CAR, anti-anti-EGFRvIII multi-chain CAR, anti-BCMA multi-chain CAR, anti-CD33 multi-chain CAR, anti-FLT3 multi-chain CAR, anti-CD70 multi-chain CAR, anti-WT1 multi-chain CAR, anti-MUC16 multi-chain CAR, anti-PRAME multi-chain CAR, anti-TSPAN10 multi-chain CAR, anti-ROR1 multi-chain CAR, anti-GD3 multi-chain CAR, anti-CT83 multi-chain CAR and mesothelin multi-chain CAR.

In a preferred embodiment, said multi-chain CAR (mcCAR) which is expressed in an immune cell initially engineered to be made hypersensitive to a specific prodrug are anti-CD123 mcCAR, or anti-CS1 mcCAR, anti-CD38 mcCAR, anti-CLL1 mcCAR or anti-Hsp70 mc CAR.

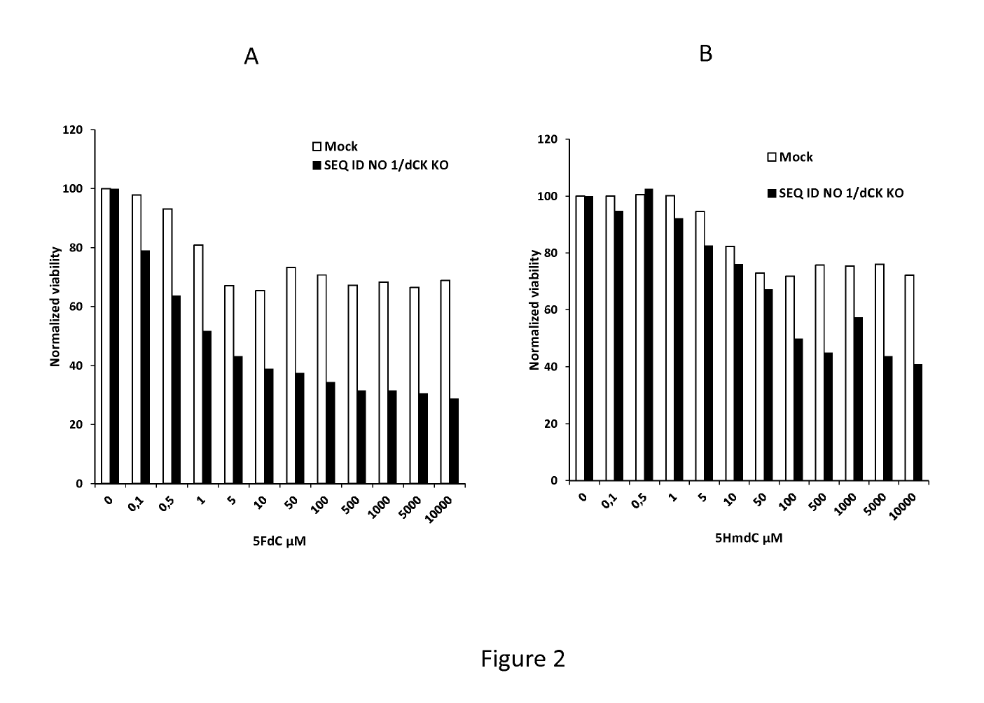

Such multi-chain CAR architectures are disclosed in WO2014/039523, especially in FIGS. 2 to 4, and from page 14 to 21, which are herein incorporated by reference.

CAR of the present invention can also be “multi-chain CARs” as previously mentioned, which means that the extracellular binding domain and the signaling domains are preferably located on different polypeptide chains, whereas co-stimulatory domains may be located on the same or a third polypeptide. Such multi-chain CARs can be derived from FcERI (Ravetch et al, 1989), by replacing the high affinity IgE binding domain of FcERI alpha chain by an extracellular ligand-binding domain such as scFv, whereas the N and/or C-termini tails of FcERI beta and/or gamma chains are fused to signal transducing domains and co-stimulatory domains respectively. The extracellular ligand binding domain has the role of redirecting T-cell specificity towards cell targets, while the signal transducing domains activate or reduce the immune cell response. The fact that the different polypeptides derive from the alpha, beta and gamma polypeptides from FcERI are transmembrane polypeptides sitting in juxtamembrane position provides a more flexible architecture to CARs, improving specificity towards the targeted molecule and reducing background activation of immune cells as described in WO2014/039523.

Allogeneic Immune Cells and Process to Make them Allogeneic

According to a particular embodiment, said specific-prodrug hypersensitive immune cells are further inactivated in their genes encoding TCRalpha or TCRbeta, to make them allogeneic.

The present invention relates also to allogeneic immunotherapy. Engraftment of allogeneic T-cells is possible by inactivating at least one gene encoding a TCR component. TCR is rendered not functional in the cells by inactivating TCR alpha gene and/or TCR beta gene(s). TCR inactivation in allogeneic T-cells avoids GvHD. Such TCR inactivation can be performed according to WO2013176915, WO201575195, WO2015136001 or WO201575195.

Immune-Checkpoint Genes

According to another particular embodiment, the present invention relates to the method for producing engineered prodrug hypersensitive immune cell, said cell being engineered further to inactivate an immune-checkpoint gene.

Thus, another particular embodiment is focused on an immune cell obtained by said above method by which the prodrug-hypersensitive immune cell is further engineered to inactivate an immune checkpoint gene. T-cell-mediated immunity includes multiple sequential steps involving the clonal selection of antigen specific cells, their activation and proliferation in secondary lymphoid tissue, their trafficking to sites of antigen and inflammation, the execution of direct effector function and the provision of help (through cytokines and membrane ligands) for a multitude of effector immune cells. Each of these steps is regulated by counterbalancing stimulatory and inhibitory signal that fine-tune the response. It will be understood by those of ordinary skill in the art, that the term “immune checkpoints” means a group of molecules expressed by T-cells. These molecules effectively serve as “brakes” to down-modulate or inhibit an immune response. Immune checkpoint molecules include, but are not limited to Programmed Death 1 (PD-1, also known as PDCD1 or CD279, accession number: NM_005018), Cytotoxic T-Lymphocyte Antigen 4 (CTLA-4, also known as CD152, GenBank accession number AF414120.1), LAG3 (also known as CD223, accession number: NM_002286.5), Tim3 (also known as HAVCR2, Gen Bank accession number: JX049979.1), BTLA (also known as CD272, accession number: NM_181780.3), BY55 (also known as CD160, GenBank accession number: CR541888.1), TIGIT (also known as VSTM3, accession number: NM_173799), LAIR1 (also known as CD305, GenBank accession number: CR542051.1, (Meyaard, Adema et al. 1997)), SIGLEC10 (GeneBank accession number: AY358337.1), 2B4 (also known as CD244, accession number: NM_001166664.1), PPP2CA, PPP2CB, PTPN6, PTPN22, CD96, CRTAM, SIGLEC7 (Nicoll, Ni et al. 1999), SIGLEC9 (Zhang, Nicoll et al. 2000; Ikehara, Ikehara et al. 2004), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF (Quigley, Pereyra et al. 2010), GUCY1A2, GUCY1A3, GUCY1B2, GUCY1B3 which directly inhibit immune cells. For example, CTLA-4 is a cell-surface protein expressed on certain CD4 and CD8 T-cells; when engaged by its ligands (B7-1 and B7-2) on antigen presenting cells, T-cell activation and effector function are inhibited. Thus the present invention relates to a method of engineering allogeneic T-cell resistant to prodrug, further comprising modifying T-cells by inactivating at least one protein involved in the immune check-point, in particular PD1 and/or CTLA-4. In a preferred embodiment, the step of inactivating at least one protein involved in the immune checkpoint is realized by expressing a rare-cutting endonuclease able to specifically cleave a target sequence within the immune checkpoint gene. In a preferred embodiment, said rare-cutting endonuclease is a TALE-nuclease. Such inactivation of immune checkpoint can be performed according to WO2014/184741.

Immunosuppressive Resistant T Cells

Allogeneic cells are rapidly rejected by the host immune system. It has been demonstrated that, allogeneic leukocytes present in non-irradiated blood products will persist for no more than 5 to 6 days (Boni, Muranski et al. 2008). Thus, to prevent rejection of allogeneic cells, the host's immune system has to be usually suppressed to some extent. However, in the case of adoptive immunotherapy the use of immunosuppressive prodrugs also have a detrimental effect on the introduced therapeutic T cells. Therefore, to effectively use an adoptive immunotherapy approach in these conditions, the introduced cells would need to be also resistant to the immunosuppressive treatment. Thus, in particular embodiment, the method according to the present invention further comprises a step of modifying T-cells to make them resistant immunosuppressive agent, preferably by inactivating at least one gene encoding a target for an immunosuppressive agent. An immunosuppressive agent is an agent that suppresses immune function by one of several mechanisms of action. In other words, an immunosuppressive agent is a role played by a compound which is exhibited by a capability to diminish the extent of an immune response. The method according to the invention allows conferring immunosuppressive resistance to T cells for immunotherapy by inactivating the target of the immunosuppressive agent in T cells. As non limiting examples, targets for immunosuppressive agent can be a receptor for an immunosuppressive agent such as: CD52, glucocorticoid receptor (GR), a FKBP family gene member and a cyclophilin family gene member. In particular embodiment, the genetic modification of the method relies on the expression, in provided cells to engineer, of one rare-cutting endonuclease such that said rare-cutting endonuclease specifically catalyzes cleavage in one targeted gene thereby inactivating said targeted gene. Said rare-cutting endonuclease can be a meganuclease, a Zinc finger nuclease or a TALE-nuclease. Such inactivation of a target of the immunosuppressive agent (ex: CD52) can be performed according to WO2013/176915.

Implementation of (Other) Suicide Genes

It may be desirable to further engineered immune cells, since engineered T-cells can expand and persist for years after administration, to include another safety mechanism—in addition to the one based on the prodrug-hypersensitivity—to allow selective deletion of administrated T-cells. Thus, in some embodiments, the method of the invention can comprises the transformation of said T-cells with a recombinant suicide gene. Said recombinant suicide gene is used to reduce the risk of direct toxicity and/or uncontrolled proliferation of said T-cells once administrated in a subject (Quintarelli C, Vera F, blood 2007; Tey S K, Dotti G., Rooney C M, boil blood marrow transplant 2007). Suicide genes enable selective deletion of transformed cells in vivo. In particular, the suicide gene has the ability to convert a non-toxic pro-prodrug into cytotoxic prodrug or to express the toxic gene expression product. In other words, “Suicide gene” is a nucleic acid coding for a product, wherein the product causes cell death by itself or in the presence of other compounds. A representative example of such a suicide gene is one which codes for thymidine kinase of herpes simplex virus. Suicide genes also include as non limiting examples caspase-9 or caspase-8. Caspase-9 can be activated using a specific chemical inducer of dimerization (CID). Suicide genes can also be polypeptides that are expressed at the surface of the cell and can make the cells sensitive to therapeutic monoclonal antibodies. As used herein “prodrug” means any compound useful in the methods of the present invention that can be converted to a toxic product. The prodrug is converted to a toxic product by the gene product of the suicide gene in the method of the present invention. A representative example of such a prodrug is ganciclovir which is converted in vivo to a toxic compound by HSV-thymidine kinase. The ganciclovir derivative subsequently is toxic to tumor cells. Other representative examples of prodrugs include acyclovir, FIAU [1-(2-deoxy-2-fluoro-β-D-arabinofuranosyl)-5-iodouracil] or 6-methoxypurine arabinoside for VZV-T K.

Activation and Expansion of T-Cells

In one embodiment, said engineered prodrug-hypersensitive immune cells in step d) of the above method of production are expanded in-vivo.

In one preferred embodiment, said engineered cells in step d) of the above method of production are expanded ex vivo or in vitro.

Whether prior to or after genetic modification of the T-cells, the T-cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. T-cells can be expanded in vitro or in vivo. Generally, the T cells of the invention are expanded by contact with an agent that stimulates a CD3 TCR complex and a co-stimulatory molecule on the surface of the T-cells to create an activation signal for the T-cell. For example, chemicals such as calcium ionophore A23187, phorbol 12-myristate 13-acetate (PMA), or mitogenic lectins like phytohemagglutinin (PHA) can be used to create an activation signal for the T-cell. As non limiting examples, T-cell populations may be stimulated in vitro such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T-cells, a ligand that binds the accessory molecule is used. For example, a population of T-cells can be contacted with an anti-CD3 antibody and an anti-CD28 antibody, under conditions appropriate for stimulating proliferation of the T-cells. To stimulate proliferation of either CD4+ T-cells or CD8+ T-cells, an anti-CD3 antibody and an anti-CD28 antibody. For example, the agents providing each signal may be in solution or coupled to a surface. As those of ordinary skill in the art can readily appreciate, the ratio of particles to cells may depend on particle size relative to the target cell.

Conditions appropriate for T-cell culture include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, 1L-4, 1L-7, GM-CSF, -10, -2, 1L-15, TGFp, IL-21 and TNF- or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, A1M-V, DMEM, MEM, a-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T-cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% C02). T cells that have been exposed to varied stimulation times may exhibit different characteristics.

Isolated Human Cell to be Engineered Using the Method of the Present Invention

The present invention relates to human cell, preferably immune cell which is engineered to have at least one gene inactivated, which is directly or indirectly involved in the metabolization, elimination or detoxification of a specific drug to make said cell hypersensitive to said specific drug.

By cell or cells is intended any eukaryotic living cells, primary cells and cell lines derived from these organisms for in vitro cultures.

The human cells which are encompassed in the scope of the present invention are those being used or having a potential for cell therapy: Embryonic stem cells (ESC), Neural stem cells (NSCs), Mesenchymal stem cells (MSC) or hematopoietic stem cells (HSCs) or induced pluripotent stem cells (iPS).

According to a preferred embodiment, said human cells to be engineered to become specific drug hypersensitive are human hematopoietic stem cells (HSCs). Human cell according to the present invention refers particularly to a cell of hematopoietic origin functionally involved in the initiation and/or execution of innate and/or adaptive immune response. This is advantageous because HSCs possess the ability to self-renew and differentiate into all types of blood cells, especially those involved in the human immune system. Thus, they can be used to treat blood and immune disorders.

According to a more preferred embodiment, said human cells particularly suitable using the method of the invention, are human primary cells.

By “primary cell” or “primary cells” are intended cells taken directly from living tissue (i.e. biopsy material) and established for growth in vitro, that have undergone very few population doublings and are therefore more representative of the main functional components and characteristics of tissues from which they are derived from, in comparison to continuous tumorigenic or artificially immortalized cell lines. As non limiting examples cell lines can be selected from the group consisting of CHO-K1 cells; HEK293 cells; Caco2 cells; U2-OS cells; NIH 3T3 cells; NSO cells; SP2 cells; CHO-S cells; DG44 cells; K-562 cells, U-937 cells; MRCS cells; IMR90 cells; Jurkat cells; HepG2 cells; HeLa cells; HT-1080 cells; HCT-116 cells; Hu-h7 cells; Huvec cells; Molt 4 cells. Primary cells are preferred since, in comparison to classical tumor cells, mimic more the physiological conditions. Moreover, it is usually advantageous to use primary cells as non-dividing cells or cells with limited doubling capacity, since genetic engineering such as transgene/shRNA expression has adverse effects on cell growth and/or viability.

According to a more preferred embodiment, said human cells particularly suitable using the method of the invention, are human immune cells, such as T-cell obtained from a donor. Said T cell according to the present invention can be derived from a stem cell. The stem cells can be adult stem cells, embryonic stem cells, more particularly non-human stem cells, cord blood stem cells, progenitor cells, bone marrow stem cells, totipotent stem cells or hematopoietic stem cells. Representative human stem cells are CD34+ cells. Said isolated cell can also be a dendritic cell, killer dendritic cell, a mast cell, a NK-cell, a B-cell or a T-cell selected from the group consisting of inflammatory T-lymphocytes, cytotoxic T-lymphocytes, regulatory T-lymphocytes or helper T-lymphocytes. In another embodiment, said cell can be derived from the group consisting of CD4+T-lymphocytes and CD8+T-lymphocytes.

Prior to expansion and genetic modification of the cells of the invention, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T-cell lines available and known to those skilled in the art, may be used. In another embodiment, said cell is preferably derived from a healthy donor. In another embodiment, said cell is part of a mixed population of cells which present different phenotypic characteristics.

Also, the present invention concerns an isolated human cell made hypersensitive to a drug obtainable by the method of production such as disclosed above.

Particularly, the engineered human cell of the invention is made hypersensitive to a specific drug by expressing or overexpressing at least one gene implicated in the drug metabolic pathway, preferably one gene encoding for an enzyme enabling the prodrug to drug conversion to confer toxicity when said cell is in presence of said prodrug.

A particular embodiment refers to an isolated human cell, preferably immune cell in which at least one of the P450 cytochrome selected in the group consisting in CYP2D6-2, CYP2C9, CYP3A4, CYP2D6-1, CYP2C19 and CYP1A2 is expressed to induce a hypersensitivity to isophosphamide and/or cyclophosphamide prodrugs.

In a more particular embodiment, an isolated human cell, preferably immune cell is engineered to express a transgene selected in the group consisting of CYP2D6-2, CYP2C9, CYP3A4, CYP2D6-1, CYP2C19, CYP2B6 and CYP1A2, said transgene sharing at least 80%, preferably 90% and more preferably 95% of identity with SEQ ID NO:2, SEQ ID NO:3, SEQ ID NO:4, SEQ ID NO:5, SEQ ID NO:6, SEQ ID NO: 8 and SEQ ID NO:7 respectively.

Another particular embodiment refers to an isolated human cell, preferably immune cell, in which at least the cytidine deaminase (CDA) is overexpressed to induce a hypersensitivity to 5fdC and/or 5hmdC prodrugs.

In a more particular embodiment, an isolated human cell, preferably immune cell is engineered to express a transgene encoding for the cytidine deaminase (CDA), said transgene sharing at least 80%, preferably 90% and more preferably 95% of identity with SEQ ID NO:1.

Another embodiment refers to an engineered human cell which is made hypersensitive to a specific drug by expressing or overexpressing at least one gene implicated in the drug metabolic pathway, preferably one gene encoding for an enzyme enabling the prodrug to drug conversion to confer toxicity when said cell is in presence of said prodrug, said cell being further genetically engineered to confer an additional drug specific hypersensitivity, the latter drug being different of that for the first hypersensitivity. Said additional hypersensitivity may be conferred by expression or overexpression of another gene implicated in a drug metabolic pathway.

An alternative to the previous embodiment is to perform said further genetically engineering human cell, preferably human immune cell, to confer drug-specific resistance to said cell, by modifying the level of expression of at least one gene, said gene being directly or indirectly involved in the metabolization, elimination or detoxification of its specific corresponding drug(s), said drug being different of the one for conferring hypersensitivity.

In a more specific embodiment, an isolated human cell, preferably immune cell is engineered to express a transgene encoding for the cytidine deaminase (CDA), said transgene sharing at least 80%, preferably 90% and more preferably 95% of identity with SEQ ID NO:1, thereby conferring hypersensitivity to 5FdC and/or 5HmdC, and said cell is further engineered to inhibit the expression of dCK gene by using a rare-cutting endonuclease targets a sequence of SEQ ID NO:17 or a sequence having at least 95% identity with the SEQ ID NO:17, thereby conferring drug resistance to purine nucleoside analog(s).

According to another embodiment, an isolated prodrug-specific hypersensitive human cell, preferably immune cell as described earlier is used as a medicament.

Therapeutic Applications

In another embodiment, said isolated (pro)drug-specific hypersensitive human cell, preferably immune cell such as T-cells obtained as previously described can be used in adoptive cell immunotherapy. In particular, said human cells, preferably immune cells, according to the present invention can be used in cell therapy or immunotherapy for treating pathologies such as cancer, infections or auto-immune disease in a patient in need thereof.

Accordingly, the present invention provides methods for treating patients in need thereof, said method comprising, for instance, one of the following steps:

(a) providing at least an isolated human cell, preferably human immune cell, which has been made hypersensitive to a specific (pro)drug, said cell being obtainable by any one of the methods previously described;

(b) Administrating said cells to said patient.

On one embodiment, said human cell, preferably human immune cell, of the invention can undergo robust in vivo expansion and can persist for an extended amount of time.

Said treatment can be ameliorating, curative or prophylactic. The invention is particularly suited for allogeneic immunotherapy, insofar as it enables the transformation of immune cells, in particular T-cells typically obtained from donors, into non-alloreactive cells by means of inactivating T-cell receptors. This may be done under standard protocols, as described in WO2013176915, incorporated herein by reference, and reproduced as many times as needed. The resulting modified T-cells are administrated to one or several patients, being made available as an “off the shelf” therapeutic product.

Cells that can be used with the disclosed methods are described in the previous sections. They may be used to treat patients diagnosed with cancer, viral infection, autoimmune disorders. Cancers that may be treated include tumors that are not vascularized, or not yet substantially vascularized, as well as vascularized tumors. The cancers may comprise nonsolid tumors (such as hematological tumors, for example, leukemias and lymphomas) or may comprise solid tumors. Types of cancers to be treated with the allogeneic human cell, preferably human immune cell hypersensitive to prodrugs of the invention include, but are not limited to carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included. In an embodiment of the present invention, childhood acute lymphoblastic leukemia (ALL) and amyotrophic myeloma leukemia (AML) diseases are typically treated by allogeneic prodrug hypersensitive cells according to the invention.

One aspect of the present invention is related to a method for transplanting human cells for the treatment of a pathology by sequential administration to a patient of:
at least one human cell which is made hypersensitive to a specific drug by selectively expressing or overexpressing at least one transgene involved in the mechanism of action of said drug and of
at least one drug to which said cells is sensitive to deplete in vivo said cells in case of occurrence of an adverse event.

In one embodiment, the invention relates to a method for treating cancer, infection or immune disease in a patient by sequential administration to a patient of:
at least one human cell which is a hematopoietic stem cell (HSC) and made hypersensitive to a specific drug by selectively expressing or overexpressing at least one transgene involved in the mechanism of action of said drug and of
at least one drug to which said cells are sensitive to deplete in vivo said cells in case of occurrence of an adverse event and/or sought modulation of the effect.

In a preferred embodiment, the method for treating cancer, infections or autoimmune diseases in a patient by sequential administration to a patient of:
at least one human immune cell, preferably T cell, which is made hypersensitive to a specific drug by selectively expressing or overexpressing at least one transgene involved in the mechanism of action of said drug, said cell being further engineered to endow a chimeric antigen receptor (CAR) specific to a cell surface antigen of said cancerous cell, infectious agent or aberrantly functioning host immune cell, and of;
at least one drug to which said cells are sensitive to deplete in vivo said cells in case of occurrence of an adverse event and/or sought modulation of the effect.

In a more preferred embodiment, said previous method comprises the administration of a CAR which is directed against a cell surface antigen specific to a cancerous cell which is a lymphoma, leukemia or solid tumor cell.

In a specific embodiment, the method for cell therapy in a patient by sequential administration to a patient of:
at least one human cell which is an immune cell made hypersensitive to cyclosphosphamide and/or isophosphamide drug by selectively expressing or overexpressing one transgene selected in the group consisting of CYP2D6-2, CYP2C9, CYP3A4, CYP2D6-1, CYP2C19 and CYP1A2,
at least cyclosphosphamide and/or isophosphamide drug to which said immune cells is sensitive to deplete in vivo said cells in case of occurrence of an adverse event and/or sought modulation of the effect.

In another specific embodiment, the method for cell therapy in a patient by sequential administration to a patient of:
at least one human cell which is an immune cell made hypersensitive to cytidine and/or deoxycytidine or analog(s) thereof by selectively expressing or overexpressing cytidine deaminase (CDA) and of;
cytidine and/or deoxycytidine or analog(s) thereof to which said cells is sensitive to deplete in vivo said cells in case of occurrence of an adverse event and/or sought modulation of the effect.

In a more specific embodiment, the method for cell therapy in a patient by sequential administration to a patient of:
at least one human cell which is an immune cell made hypersensitive to 5FdC and/or 5HmdC drug by selectively expressing or overexpressing cytidine deaminase (CDA) and of;
at least 5FdC and/or 5HmdC drug to which said immune cells is sensitive to deplete in vivo said cells in case of occurrence of an adverse event and/or sought modulation of the effect.

In a specific embodiment, a method for treating cancer sensitive to a purine nucleoside analog in a patient by sequential administration to a patient of:
at least one human cell which is a immune cell and made hypersensitive to 5FdC and/or 5HmdC drug, or to cyclosphosphamide and/or isophosphamide, by selectively expressing or overexpressing cytidine deaminase (CDA);
optionally, a purine nucleoside analog drug to which said engineered cell is resistant by inactivating dCK gene; said drug being used to treat cancerous cells; and of
5FdC and/or 5HmdC drug, or cyclosphosphamide and/or isophosphamide, to which said cells are sensitive to deplete in vivo said cells in case of occurrence of an adverse event and/or sought modulation of the effect,

and wherein the administrations of said engineered immune cell and the purine nucleoside analog are concomitant or successive regardless of the order.

Said previous purine nucleoside analog drug is preferably clofarabine, fludarabine and/or cladribine.

It can be a treatment in combination with one or more therapies against cancer selected from the group of antibodies therapy, chemotherapy, cytokines therapy, dendritic cell therapy, gene therapy, hormone therapy, laser light therapy and radiation therapy.

According to a preferred embodiment of the invention, said treatment is administrated into patients undergoing an immunosuppressive treatment. The present invention preferably relies on cells or population of cells, which have been made hypersensitive to at least one prodrug agent according to the present invention due to the inactivation of a prodrug sensitizing gene. In this aspect, the prodrug treatment should help the selection and expansion of the T-cells according to the invention within the patient.

The administration of the cells or population of cells according to the present invention may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, intracranially, by intravenous or intralymphatic injection, or intraperitoneally. In one embodiment, the cell compositions of the present invention are preferably administered by intravenous injection.

The administration of the cells or population of cells, particularly of immune cells, can consist of the administration of 10³-10¹⁰ cells per kg body weight, preferably 10⁵ to 10⁶ cells/kg body weight including all integer values of cell numbers within those ranges. The cells or population of cells can be administrated in one or more doses. In another embodiment, said effective amount of cells are administrated as a single dose. In another embodiment, said effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, said effective amount of cells or pharmaceutical composition comprising those cells are administrated parenterally. Said administration can be an intravenous administration. Said administration can be directly done by injection within a tumor.

In certain embodiments of the present invention, cells are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or nataliziimab treatment for MS patients or efaliztimab treatment for psoriasis patients or other treatments for PML patients. In further embodiments, the T-cells of the invention may be used in combination with chemotherapy, radiation, immunosuppressive agents, such as cyclosporin, azathioprine, mycophenolate, and FK506, antibodies, or other immunoablative agents such as CAMPATH, anti-CD3 antibodies or other antibody therapies, cytoxin, fludaribine, cyclosporin, FK506, rapamycin, mycophenolic acid, steroids, FR901228, cytokines, and irradiation. These prodrugs inhibit either the calcium dependent phosphatase calcineurin (cyclosporine and FK506) or inhibit the p7056 kinase that is important for growth factor induced signaling (rapamycin) (Liu et al., Cell 66:807-815, 1 1; Henderson et al., Immun. 73:316-321, 1991; Bierer et al., Citrr. Opin. mm n. 5:763-773, 93). In a further embodiment, the cell compositions of the present invention are administered to a patient in conjunction with (e.g., before, simultaneously or following) bone marrow transplantation, T-cell ablative therapy using either chemotherapy agents such as, fludarabine, external-beam radiation therapy (XRT), cyclophosphamide, or antibodies such as OKT3 or CAMPATH, In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

Pharmaceutical Composition

The isolated drug specific hypersensitive human cells, preferably immune cells (ie T-cells), of the present invention may be administered either alone, or as a pharmaceutical composition in combination with diluents and/or with other components such as IL-2 or other cytokines or cell populations. Briefly, pharmaceutical compositions of the present invention may comprise T-cells as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g. aluminum hydroxide); and preservatives. Compositions of the present invention are preferably formulated for intravenous administration. Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

### Definitions

In the description above, a number of terms are used extensively. The following definitions are provided to facilitate understanding of the present embodiments.
Amino acid residues in a polypeptide sequence are designated herein according to the one-letter code, in which, for example, Q means Gln or Glutamine residue, R means Arg or Arginine residue and D means Asp or Aspartic acid residue.
Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
As used herein, “nucleic acid” or “nucleic acid molecule” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Nucleic acids can be either single stranded or double stranded.
By “genome” it is meant the entire genetic material contained in a cell such as nuclear genome, chloroplastic genome, mitochondrial genome.
By “mutation” is intended the substitution, deletion, insertion of one or more nucleotides/amino acids in a polynucleotide (cDNA, gene) or a polypeptide sequence. Said mutation can affect the coding sequence of a gene or its regulatory sequence. It may also affect the structure of the genomic sequence or the structure/stability of the encoded mRNA.
The term “rare-cutting endonuclease” refers to a wild type or variant enzyme capable of catalyzing the hydrolysis (cleavage) of bonds between nucleic acids within a DNA or RNA molecule, preferably a DNA molecule. Particularly, said nuclease can be an endonuclease, more preferably a rare-cutting endonuclease which is highly specific, recognizing nucleic acid target sites ranging from 10 to 45 base pairs (bp) in length, usually ranging from 10 to 35 base pairs in length. The endonuclease according to the present invention recognizes and cleaves nucleic acid at specific polynucleotide sequences, further referred to as “target sequence”. The rare-cutting endonuclease can recognize and generate a single- or double-strand break at specific polynucleotides sequences.

“TALE-nuclease” or “MBBBD-nuclease” refers to engineered proteins resulting from the fusion of a DNA binding domain typically derived from Transcription Activator like Effector proteins (TALE) or MBBBD binding domain, with an endonuclease catalytic domain. Such catalytic domain is preferably a nuclease domain and more preferably a domain having endonuclease activity, like for instance I-Tevl, ColE7, NucA and Fok-I. In a particular embodiment, said nuclease is a monomeric TALE-Nuclease or MBBBD-nuclease. A monomeric Nuclease is a nuclease that does not require dimerization for specific recognition and cleavage, such as the fusions of engineered DNA binding domain with the catalytic domain of I-Tevl described in WO2012138927. In another particular embodiment, said rare-cutting endonuclease is a dimeric TALE-nuclease or MBBBD-nuclease, preferably comprising a DNA binding domain fused to FokI. TALE-nuclease have been already described and used to stimulate gene targeting and gene modifications (Boch, Scholze et al. 2009; Moscou and Bogdanove 2009; Christian, Cermak et al. 2010). Such engineered TALE-nucleases are commercially available under the trade name TALEN™ (Cellectis, 8 rue de la Croix Jarry, 75013 Paris, France).
The term “cleavage” refers to the breakage of the covalent backbone of a polynucleotide. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. Double stranded DNA, RNA, or DNA/RNA hybrid cleavage can result in the production of either blunt ends or staggered ends.
Because some variability may arise from the genomic data from which these polypeptides derive, and also to take into account the possibility to substitute some of the amino acids present in these polypeptides without significant loss of activity (functional variants), the invention encompasses polypeptides variants of the above polypeptides that share at least 70%, preferably at least 80%, more preferably at least 90% and even more preferably at least 95% identity with the sequences provided in this patent application.
“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated;
«knockout» means that the gene is mutated to that extend it cannot be expressed anymore;
“TRAC” refers to “T cell receptor alpha constant» and corresponds to TCRα subunit constant gene.

In addition to the preceding features, the invention comprises further features which will emerge from the following examples illustrating the method of engineering prodrug hypersensitive T-cells for immunotherapy, as well as to the appended drawings.

General Methods

Primary T-Cell Cultures

T cells were purified from Buffy coat samples provided by EFS (Etablissement Français du Sang, Paris, France) using Ficoll gradient density medium. The PBMC layer was recovered and T cells were purified using a commercially available T-cell enrichment kit. Purified T cells were activated in X-Vivo™-15 medium (Lonza) supplemented with 20 ng/mL Human IL-2, 5% Human, and Dynabeads Human T activator CD3/CD28 at a bead:cell ratio 1:1 (Life Technologies).

CAR mRNA Transfection

Transfections are typically done at Day 4 or Day 11 after T-cell purification and activation. 5 millions of cells were transfected with 15 μg of mRNA encoding the different CAR constructs. CAR mRNAs are usually produced using T7 mRNA polymerase and transfections done using Cytopulse technology, for instance by applying two 0.1 mS pulses at 3000V/cm followed by four 0.2 mS pulses at 325V/cm in 0.4 cm gap cuvettes in a final volume of 200 μl of “Cytoporation buffer T” (BTX Harvard Apparatus). Cells were immediately diluted in X-Vivo™-15 media and incubated at 37° C. with 5% CO₂. IL-2 was added 2 h after electroporation at 20 ng/mL.

T-Cell Transduction

Transduction of T-cells with recombinant lentiviral vectors expression the CAR is typically carried out three days after T-cell purification/activation. Transductions were carried out at a multiplicity of infection of 5, using 10⁶ cells per transduction. CAR detection at the surface of T-cells was done using a recombinant protein consisting on the fusion of the extracellular domain of the human protein such as CD123 or CD19 together with a murine IgG1 Fc fragment (produced by LakePharma). Binding of this protein to the CAR molecule was detected with a PE-conjugated secondary antibody (Jackson Immunoresearch) targeting the mouse Fc portion of the protein, and analyzed by flow cytometry.

Degranulation Assay (CD107a Mobilization)

T-cells were incubated in 96-well plates (40,000 cells/well), together with an equal amount of cells expressing various levels of the CD123 protein. Co-cultures were maintained in a final volume of 100 μl of X-Vivo™-15 medium (Lonza) for 6 hours at 37° C. with 5% CO₂. CD107a staining was done during cell stimulation, by the addition of a fluorescent anti-CD107a antibody at the beginning of the co-culture, together with 1 μg/ml of anti-CD49d, 1 μg/ml of anti-CD28, and 1× Monensin solution. After the 6 h incubation period, cells were stained with a fixable viability dye and fluorochrome-conjugated anti-CD8 and analyzed by flow cytometry. The degranulation activity was determined as the % of CD8+/CD107a+ cells, and by determining the mean fluorescence intensity signal (MFI) for CD107a staining among CD8+ cells. Degranulation assays were carried out 24 h after mRNA transfection.

IFN Gamma Release Assay

T-cells were incubated in 96-well plates (40,000 cells/well), together with cell lines expressing various levels of the CD123 protein. Co-cultures were maintained in a final volume of 100 μl of X-Vivo™-15 medium (Lonza) for 24 hours at 37° C. with 5% CO₂. After this incubation period the plates were centrifuged at 1500 rpm for 5 minutes and the supernatants were recovered in a new plate. IFN gamma detection in the cell culture supernatants was done by ELISA assay. The IFN gamma release assays were carried by starting the cell co-cultures 24 h after mRNA transfection.

Cytotoxicity Assay

T-cells were incubated in 96-well plates (100,000 cells/well), together with 10,000 target cells (expressing the CAR-T cell target protein) and 10,000 control (not expressing the CAR-T cell target protein) cells in the same well. Target and control cells were labelled with fluorescent intracellular dyes (CFSE or Cell Trace Violet) before co-culturing them with CAR+ T-cells. The co-cultures were incubated for 4 hours at 37° C. with 5% CO₂. After this incubation period, cells were labelled with a fixable viability dye and analyzed by flow cytometry. Viability of each cellular population (target cells or control cells which do not express the targeted antigen surface protein) was determined and the % of specific cell lysis was calculated. Cytotoxicity assays were carried out 48 h after mRNA transfection.

TALE-Nuclease-Mediated Gene Inactivation

To inactivate a gene such as one described here (such as drug resistance gene, ie dCk, or TCR, or immune checkpoint by instance), two pairs of TALE-nucleases were designed for each gene, assembled and validated by sequencing. Once validated, mRNAs encoding the two TALE-nucleases were produced, polyadenylated and used to electroporate T cells using pulse agile technology (5 or 10 μg of TALE-nuclease mRNA left and right were used) such as described in the WO 2013/176915. A cold temperature shock are usually performed by incubating T cells at 30° C. immediately after electroporation and for 24 hours. A reactivation (12.5 μl beads/10⁶ cells) was performed at D8 (8 days after the electroporation). The resulting T cells were allowed to grow and eventually characterized genotypically (by Endo T7 assay and deep sequencing at the gene loci to target) as well as phenotypically. Their phenotypical characterization consisted of (i), checking their ability to grow in the presence or absence of drug (ii), determining the IC₅₀ of corresponding drugs (such as PNAs, clofarabine and fludarabine for dCK gene), toward T cells and (iii), determining the extent of TRAC inactivation by FACS analysis when double KO is performed.

Genotypic Characterization of T Cells Having Undergone a KO in a Drug Metabolization-Related Gene

To assess the efficiency of drug metaboliztion-related gene inactivation, cells transfected with either 5 or 10 μg of TALE-nuclease mRNA were grown for 4 days (D4, 4 days after electroporation) and collected to perform T7 assays at the locus of interest. The T7 assay protocol is described in Reyon, D., Tsai, S. Q., Khayter, C., Foden, J. A., Sander, J. D., and Joung, J. K. (2012) FLASH assembly of TALE-nucleases for high-throughput genome editing. Nat Biotechnologies.

Determination of Growth Rate of T Cells with a KO in the Gene of Interest (GOI)

T cells with a GOI-KO are tested for their growth rate and for their reactivation with respect to WT cells.

Selection of GOI-KO T Cell in the Presence of the Drug

GOI KO or WT T cells are typically allowed to grow from D8 to D13 and then incubated with or without corresponding drug to which KO T cells are made resistant until D18. Cells were collected at D8 (before drug addition) and at D18 (after drug incubation) and were used to perform an endo T7 assay.

Determination of IC50 for the Drug on GOI KO T Cells Versus WT T Cells

To further investigate the ability of T cells to resist to the drug, IC50 for this drug was determined on GOI KO and WT T cells. The cells were collected 3 days after transfection were incubated for 2 days in media having different concentrations of said drug. At the end of drug incubation, viability of T cells was determined by FACS analysis.

### Example 1: CDA Overexpression and dCK Inactivation in T Cell to Confer Respectively Hypersensitivity to Cytidine Analogs and Resistance to Clofarabine

The inventors have sought to engineer 5-hydroxymethyl-2′-deoxycytidine (5hmdC) or 5-formyl-2′ deoxycytidine (5fdC) sensitivity by combining the genetic inactivation of dCK with transgenic expression of CDA.

Experimental Protocols

CDA Expression

To test the ability of CDA expression to endow primary T cell with hypersensitivity to hypomethylated agents 5hmdC and 5FdC, primary T cells were transfected with 40 μg of mRNA encoding a chimeric construction consisting of CDA fused to a BFP reported via a 2A self-cleaving peptide (SEQ ID NO. 9). One day post transfection, cells were recovered and analyzed by flow cytometry for BFP expression.

TALE-Nuclease-Mediated Inactivation of dCK

To inactivate dCK, two pairs of dCK TALE-nucleases were designed, assembled and validated by sequencing; subsequent work was performed only with the pair named TALE-nuclease dCK2 and having SEQ ID NO:18 and SEQ ID NO:19. The details regarding the dCK gene overall architecture (exons and introns) and the sequences of TALE-nuclease target sites located in the exon 2 are indicated in application WO201575195.

The dCK target sequence for the TALE-nuclease dCK2 pair corresponds to SEQ ID N^{o} 17.

Once validated, mRNAs encoding the two TALE-nucleases were produced, polyadenylated and used to electroporate T cells using pulse agile technology (5 or 10 μg of TALE-nuclease mRNA left and right were used) such as described in the WO 2013/176915. A cold temperature shock was performed by incubating T cells at 30° C. immediately after electroporation and for 24 hours. A reactivation (12.5 μl beads/10⁶ cells) was performed at D8 (8 days after the electroporation).

The resulting T cells were allowed to grow and eventually characterized genotypically (by Endo T7 assay and deep sequencing at dCK) as well as phenotypically. Their phenotypical characterization consisted of checking their ability to grow in the presence or absence of prodrug and determining the IC₅₀ toward T cells.

Genotypic Characterization of dCK KO T Cells

To assess the efficiency of dCK gene inactivation, cells transfected with either 5 or 10 μg of TALE-nuclease mRNA were grown for 4 days (D4, 4 days after electroporation) and collected to perform T7 assays at the dCK locus. The sequences for the primers used in these T7 assays correspond to the ones used in WO201575195. The T7 assay protocol is described in Reyon, D., Tsai, S. Q., Khayter, C., Foden, J. A., Sander, J. D., and Joung, J. K. (2012) FLASH assembly of TALE-nucleases for high-throughput genome editing. Nat Biotechnologies.

Determination of Growth Rate of KO T Cells dCK KO cells display similar growth rate with respect to WT cells. In addition, they could be reactivated at D8 with the same efficiency than WT T cells.

Determination of IC50 for 5fdC on dCK KO T Cells Versus WT T Cells

To further investigate the ability of T cells to be sensitive to 5fdC, IC50 for this prodrug was determined on dCK KO and WT T cells. The cells were collected 3 days after transfection were incubated for 2 days in the presence of increasing concentration of 5fdC (0 to 10 mM). At the end of 5fdC incubation, viability of T cells was determined by FACS analysis.

Results

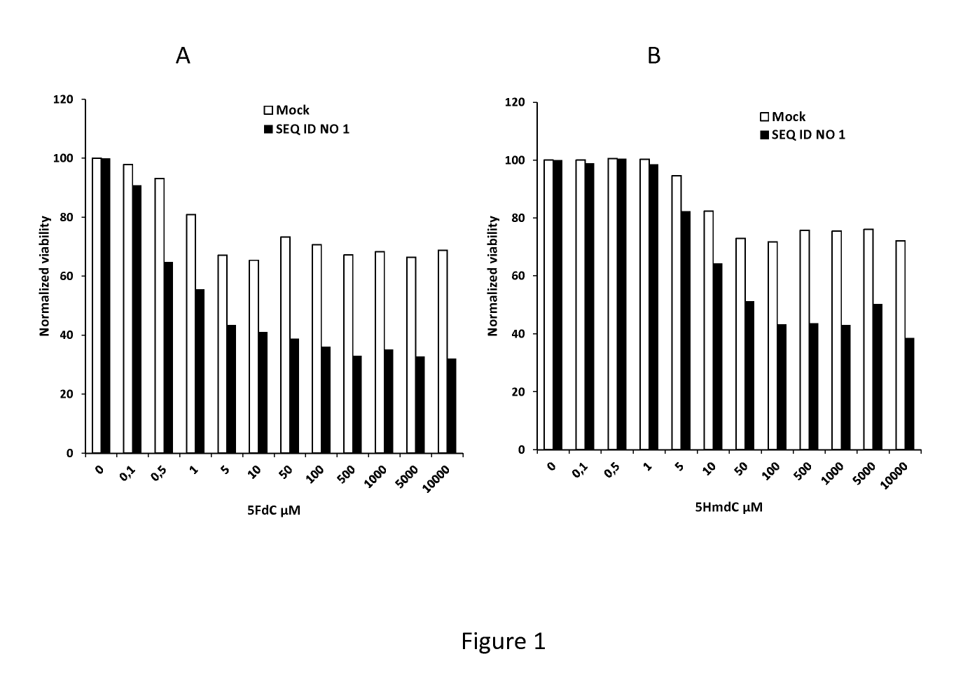

The results showed that 68% of cells expressed BFP indicating that transfection successfully enabled expression of CDA-BFP construction. Transfected cells were incubated in the presence of increasing concentration of 5hmdC or 5FdC for 48H. At the end of incubation, cell viability was determined by flow cytometry. Our results showed an increase of sensitivity transfected T cells with respect to wild type cells toward both components. FIG. 1 reports the results obtained from the expression of CDA, it is shown that transfected T cells are enabled to metabolize 5hmDC and SFDC into toxic components thus out-competing the opposite activity of dCK.

FIG. 2 presents the results to show whether primary T cells having undergone dCK KO can still endow resistance to purine nucleotide analogues as well as with hypersensitivity toward 5hmDC and SFDC. One day post transfection, dCK KO T cells were recovered and analyzed by flow cytometry for BFP expression. The results shown in FIG. 2 that about 56% of cells expressed BFP indicating once again that transfection successfully enabled expression of CDA-BFP construction. As described earlier, transfected cells were incubated in the presence of increasing concentration of 5hmdC or 5FdC for 48H and at the end of incubation, cell viability was determined by flow cytometry. In FIG. 2 is clearly apparent an increase of specific prodrug hypersensitivity transfected T cells with respect to wild type cells toward both components.

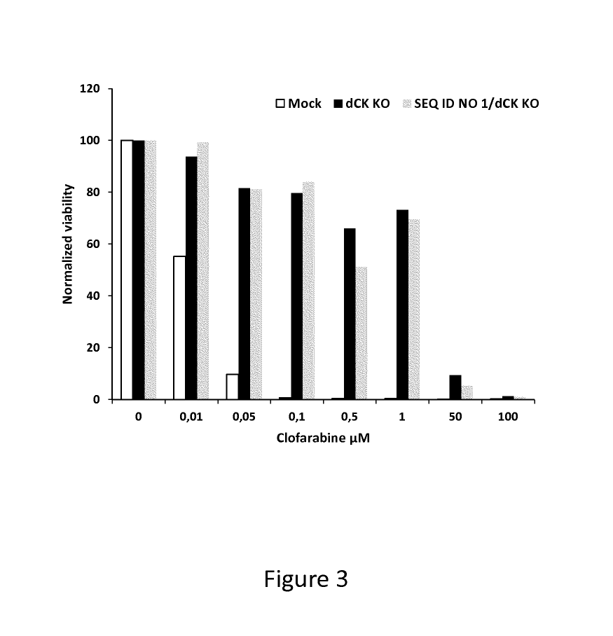

In addition the same cells were incubated for 48H before determining their viability. Our results showed that T cells KO dCK expressing CDA-BFP and T cells KO dCK showed similar resistance properties with respect to clofarabine (FIG. 3). Taken together, T cells dCK KO expressing CDA are able to resist to clofarabine while being hypersensitive to the epigenetically modified cytosine compounds named 5hmdC and 5FdC.

### Example 2: Overexpression of CYP2D6-2 in T Cell to Confer Hypersensitivity to Isophosphamide and/or Cyclophosphamide

To test the ability of CYP2D6-2 expression to endow primary T cell with hypersensitivity to isophosphamide and/or cyclophosphamide, primary T cells were transfected with 40 μg of mRNA encoding a chimeric construction consisting of CYP2D6 isoform 2 fused to a BFP reported via a 2A self-cleaving peptide (SEQ ID NO. 10). One day post transfection, cells were recovered and analyzed by flow cytometry for BFP expression.

### Example 3: Overexpression of CYP2C9 in T Cell to Confer Hypersensitivity to Isophosphamide and/or Cyclophosphamide

To test the ability of CYP2C9 expression to endow primary T cell with hypersensitivity to isophosphamide and/or cyclophosphamide, primary T cells were transfected with 40 μg of mRNA encoding a chimeric construction consisting of CYP2C9 isoform 2 fused to a BFP reported via a 2A self-cleaving peptide (SEQ ID NO. 11). One day post transfection, cells were recovered and analyzed by flow cytometry for BFP expression.

### Example 4: Overexpression of CYP3A4 in T Cell to Confer Hypersensitivity to Isophosphamide and/or Cyclophosphamide

To test the ability of CYP3A4 expression to endow primary T cell with hypersensitivity to isophosphamide and/or cyclophosphamide, primary T cells were transfected with 40 μg of mRNA encoding a chimeric construction consisting of CYP3A4 fused to a BFP reported via a 2A self-cleaving peptide (SEQ ID NO. 12). One day post transfection, cells were recovered and analyzed by flow cytometry for BFP expression.

### Example 5: Overexpression of CYP2D6-1 in T Cell to Confer Hypersensitivity to Isophosphamide and/or Cyclophosphamide

To test the ability of CYP2D6 isoform 1 expression to endow primary T cell with hypersensitivity to isophosphamide and/or cyclophosphamide, primary T cells were transfected with 40 μg of mRNA encoding a chimeric construction consisting of CYP2D6 isoform 1 fused to a BFP reported via a 2A self-cleaving peptide (SEQ ID NO. 13). One day post transfection, cells were recovered and analyzed by flow cytometry for BFP expression.

### Example 6: Overexpression of CYP2C19 in T Cell to Confer Hypersensitivity to Isophosphamide and/or Cyclophosphamide

To test the ability of CYP2C19 expression to endow primary T cell with hypersensitivity to isophosphamide and/or cyclophosphamide, primary T cells were transfected with 40 μg of mRNA encoding a chimeric construction consisting of CYP2C19 fused to a BFP reported via a 2A self-cleaving peptide (SEQ ID NO. 14). One day post transfection, cells were recovered and analyzed by flow cytometry for BFP expression.

### Example 7: Overexpression of CYP1A2 in T Cell to Confer Hypersensitivity to Isophosphamide and/or Cyclophosphamide

To test the ability of CYP1A2 expression to endow primary T cell with hypersensitivity to isophosphamide and/or cyclophosphamide, primary T cells were transfected with 40 μg of mRNA encoding a chimeric construction consisting of CYP1A2 fused to a BFP reported via a 2A self-cleaving peptide (SEQ ID NO. 15). One day post transfection, cells were recovered and analyzed by flow cytometry for BFP expression.

### Example 8: Overexpression of CYP2B6 in T Cell to Confer Hypersensitivity to Isophosphamide and/or Cyclophosphamide

To test the ability of CYP1A2 expression to endow primary T cell with hypersensitivity to isophosphamide and/or cyclophosphamide, primary T cells were transfected with 40 μg of mRNA encoding a chimeric construction consisting of CYP2B6 fused to a BFP reported via a 2A self-cleaving peptide (SEQ ID NO. 16). One day post transfection, cells were recovered and analyzed by flow cytometry for BFP expression.

### REFERENCES

Betts, M. R., J. M. Brenchley, et al. (2003). “Sensitive and viable identification of antigen-specific CD8+ T cells by a flow cytometric assay for degranulation.” J Immunol Methods 281(1-2): 65-78.
Boch, J., H. Scholze, et al. (2009). “Breaking the code of DNA binding specificity of TAL-type III effectors.” Science 326(5959): 1509-12.
Cermak, T., E. L. Doyle, et al. (2011). “Efficient design and assembly of custom TALEN and other TAL effector-based constructs for DNA targeting.” Nucleic Acids Res 39(12): e82.
Chang T K, Yu L, Goldstein J A, Waxman D J. 1997 “Identification of the polymorphically expressed CYP2C19 and the wild-type CYP2C9-ILE359 allele as low-Km catalysts of cyclophosphamide and ifosfamide activation” Pharmacogenetics. 7(3):211-21.
Chang T K, Weber G F, Crespi C L, Waxman D J. 1993 “Differential activation of cyclophosphamide and ifosphamide by cytochromes P-450 2B and 3A in human liver microsomes”, Cancer Res. 53(23):5629-37.
Christian, M., T. Cermak, et al. (2010). “Targeting DNA double-strand breaks with TAL effector nucleases.” Genetics 186(2): 757-61.
Cong, L., F. A. Ran, et al. (2013). “Multiplex genome engineering using CRISPR/Cas systems.” Science 339(6121): 819-23.
Critchlow, S. E. and S. P. Jackson (1998). “DNA end-joining: from yeast to man.” Trends Biochem Sci 23(10): 394-8.
Deltcheva, E., K. Chylinski, et al. (2011). “CRISPR RNA maturation by trans-encoded small RNA and host factor RNase III.” Nature 471(7340): 602-7.
Deng, D., C. Yan, et al. (2012). “Structural basis for sequence-specific recognition of DNA by TAL effectors.” Science 335(6069): 720-3.
Garneau, J. E., M. E. Dupuis, et al. (2010). “The CRISPR/Cas bacterial immune system cleaves bacteriophage and plasmid DNA.” Nature 468(7320): 67-71.
Gasiunas, G., R. Barrangou, et al. (2012). “Cas9-crRNA ribonucleoprotein complex mediates specific DNA cleavage for adaptive immunity in bacteria.” Proc Natl Acad Sci USA 109(39): E2579-86.
Geissler, R., H. Scholze, et al. (2011). “Transcriptional activators of human genes with programmable DNA-specificity.” PLoS One 6(5): e19509.
Huang, P., A. Xiao, et al. (2011). “Heritable gene targeting in zebrafish using customized TALENs.” Nat Biotechnol 29(8): 699-700.
Jena, B., G. Dotti, et al. (2010). “Redirecting T-cell specificity by introducing a tumor-specific chimeric antigen receptor.” Blood 116(7): 1035-44.
Jinek, M., K. Chylinski, et al. (2012). “A programmable dual-RNA-guided DNA endonuclease in adaptive bacterial immunity.” Science 337(6096): 816-21.
Li, L., M. J. Piatek, et al. (2012). “Rapid and highly efficient construction of TALE-based transcriptional regulators and nucleases for genome modification.” Plant Mol Biol 78(4-5): 407-16.
Li, T., S. Huang, et al. (2011). “Modularly assembled designer TAL effector nucleases for targeted gene knockout and gene replacement in eukaryotes.” Nucleic Acids Res 39(14): 6315-25.
Mahfouz, M. M., L. Li, et al. (2012). “Targeted transcriptional repression using a chimeric TALE-SRDX repressor protein.” Plant Mol Biol 78(3): 311-21.
Mahfouz, M. M., L. Li, et al. (2011). “De novo-engineered transcription activator-like effector (TALE) hybrid nuclease with novel DNA binding specificity creates double-strand breaks.” Proc Natl Acad Sci USA 108(6): 2623-8.
Mali, P., L. Yang, et al. (2013). “RNA-guided human genome engineering via Cas9.” Science 339(6121): 823-6.
Meyaard, L., G. J. Adema, et al. (1997). “LAIR-1, a novel inhibitory receptor expressed on human mononuclear leukocytes.” Immunity 7(2): 283-90.
Miller, J. C., S. Tan, et al. (2011). “A TALE nuclease architecture for efficient genome editing.” Nat Biotechnol 29(2): 143-8.
Morbitzer, R., P. Romer, et al. (2011). “Regulation of selected genome loci using de novo-engineered transcription activator-like effector (TALE)-type transcription factors.” Proc Natl Acad Sci USA 107(50): 21617-22.
Moscou, M. J. and A. J. Bogdanove (2009). “A simple cipher governs DNA recognition by TAL effectors.” Science 326(5959): 1501.
Mussolino, C., R. Morbitzer, et al. (2011). “A novel TALE nuclease scaffold enables high genome editing activity in combination with low toxicity.” Nucleic Acids Res 39(21): 9283-93.
Park, T. S., S. A. Rosenberg, et al. (2011). “Treating cancer with genetically engineered T cells.” Trends Biotechnol 29(11): 550-7.
Pavlos R, Mallal S Ostrov D, Buus S, Metushi M, Peters B, and Phillips E, 2015, “T Cell-Mediated Hypersensitivity Reactions to Prodrugs”, Annu Rev Med.; 66: 439-454
Quigley, M., F. Pereyra, et al. (2010). “Transcriptional analysis of HIV-specific CD8+ T cells shows that PD-1 inhibits T cell function by upregulating BATF.” Nat Med 16(10): 1147-51.
Sander, J. D., L. Cade, et al. (2011). “Targeted gene disruption in somatic zebrafish cells using engineered TALENs.” Nat Biotechnol 29(8): 697-8.
Sorek, R., C. M. Lawrence, et al. (2013). “CRISPR-mediated Adaptive Immune Systems in Bacteria and Archaea.” Annu Rev Biochem.
Stoddard, B. L. (2005). “Homing endonuclease structure and function.” Q Rev Biophys 38(1): 49-95.
Sugimoto, Y., S. Tsukahara, et al. (2003). “Prodrug-selected co-expression of P-glycoprotein and gp91 in vivo from an MDR1-bicistronic retrovirus vector Ha-MDR-IRES-gp91.” J Gene Med 5(5): 366-76.
Takebe, N., S. C. Zhao, et al. (2001). “Generation of dual resistance to 4-hydroperoxycyclophosphamide and methotrexate by retroviral transfer of the human aldehyde dehydrogenase class 1 gene and a mutated dihydrofolate reductase gene.” Mol Ther 3(1): 88-96.
Tesson, L., C. Usal, et al. (2011). “Knockout rats generated by embryo microinjection of TALENs.” Nat Biotechnol 29(8): 695-6.
Weber, E., R. Gruetzner, et al. (2011). “Assembly of designer TAL effectors by Golden Gate cloning.” PLoS One 6(5): e19722.
Yam, P., M. Jensen, et al. (2006). “Ex vivo selection and expansion of cells based on expression of a mutated inosine monophosphate dehydrogenase 2 after HIV vector transduction: effects on lymphocytes, monocytes, and CD34+ stem cells.” Mol Ther 14(2): 236-44.
Zhang, F., L. Cong, et al. (2011). “Efficient construction of sequence-specific TAL effectors for modulating mammalian transcription.” Nat Biotechnol 29(2): 149-53.
Zhang, J. Q., G. Nicoll, et al. (2000). “Siglec-9, a novel sialic acid binding member of the immunoglobulin superfamily expressed broadly on human blood leukocytes.” J Biol Chem 275(29): 22121-6.

1. A method of producing human cell that may be depleted in-vivo as part of a cell therapy or immunotherapy treatment, said method comprising:
(a) providing a human cell;
(b) inducing drug hypersensitivity into said cell by selectively overexpressing an endogenous gene or a transgene involved in the toxicity of a prodrug to such cell,
wherein said endogenous gene or transgene is CDA encoding cytosine deaminase or selected from the P450 cytochromes family consisting of CYP2D6-2, CYP2C9, CYP3A4, CYP2D6-1, CYP2C19 and CYP1A2; and
(c) expanding said engineered cell obtained in step b).

2. The method according to claim 1, wherein a population of cells is produced, which comprises at least 95% of human cells that have become hypersensitive to the prodrug.

3. The method of claim 1, wherein the expression of said endogenous gene or transgene in step (b) mediates the chemical conversion of said prodrug to an active drug which is toxic to said cell.

4. The method of claim 1, wherein said human cell is a human immune cell.

5. The method of claim 1, wherein said human cell is a human primary cell.

6. The method of claim 1, wherein said immune cell is a T cell.

7. The method of claim 1, wherein said endogenous gene or transgene is CDA, making the cell hypersensitive to deoxycytidine analog(s).

8. The method according to claim 7, wherein said analogs are 5fdC and 5hmdC prodrugs.

9. (canceled)

10. The method according to claim 1, wherein said endogenous gene or transgene is selected from the group consisting of the P450 cytochromes family consisting of CYP2D6-2, CYP2C9, CYP3A4, CYP2D6-1, CYP2C19 and CYP1A2 making the cell hypersensitive to cyclophosphamide or isophosphamide.

11-18. (canceled)

19. The method of claim 1, wherein said transgene is introduced into the cell using a viral vector delivery vector.

20. The method of claim 19, wherein said viral vector is a lentiviral vector.

21-26. (canceled)

27. The method of claim 1, wherein said method further comprises the step of expressing a Chimeric Antigen Receptor (CAR) in said cell.

28. The method of claim 27, wherein said chimeric antigen receptor is directed against CD123+, CD19+, CS1+, CD38+, ROR11+, CLL1+, hsp70+, CD22+, EGFRvIII+, BCMA+, CD33+, FLT3+, CD70+, WT1+, MUC16+, PRAME+, TSPAN10+, ROR1+, GD3+, CT83+, or mesothelin+ antigens.

29. (canceled)

30. The method of claim 1, wherein said cells are further inactivated in their genes encoding TCRalpha or TCRbeta.

31. (canceled)

32. An isolated human cell or population of cells made hypersensitive to a drug, obtainable by the method claim 1.

33. (canceled)

34. A pharmaceutical composition comprising at least one isolated cell or population of cells according to claim 32.

35-44. (canceled)


SEQUENCE LISTING (27 sequences)

SEQ ID NO: 1 - protein, 146 aa, Homo sapiens [sequence omitted]

SEQ ID NO: 2 - protein, 497 aa, Homo sapiens
  P450 isozyme CYP2D6_2
MGLEALVPLAVIVAIFLLLVDLMHRRQRWAARYPPGPLPLPGLGNLLHVDFQNTPYCFDQLRRRFGDVFSLQLAWTPVVVLNGLAAVREALVTHGEDTADRPPVPITQILGFGPRSQGVFLARYGPAWREQRRFSVSTLRNLGLGKKSLEQWVTEEAACLCAAFANHSGRPFRPNGLLDKAVSNVIASLTCGRRFEYDDPRFLRLLDLAQEGLKEESGFLREVLNAVPVLLHIPALAGKVLRFQKAFLTQLDELLTEHRMTWDPAQPPRDLTEAFLAEMEKAKGNPESSFNDENLCIVVADLFSAGMVTTSTTLAWGLLLMILHPDVQRRVQQEIDDVIGQVRRPEMGDQAHMPYTTAVIHEVQRFGDIVPLGVTHMTSRDIEVQGFRIPKGTTLITNLSSVLKDEAVWEKPFRFHPEHFLDAQGHFVKPEAFLPFSAGRRACLGEPLARMELFLFFTSLLQHFSFSVPTGQPRPSHHGVFAFLVTPSPYELCAVPR

SEQ ID NO: 3 - protein, 490 aa, Homo sapiens [sequence omitted]

SEQ ID NO: 4 - protein, 503 aa, Homo sapiens
  P450 isozyme CYP3A4
MALIPDLAMETWLLLAVSLVLLYLYGTHSHGLFKKLGIPGPTPLPFLGNILSYHKGFCMFDMECHKKYGKVWGFYDGQQPVLAITDPDMIKTVLVKECYSVFTNRRPFGPVGFMKSAISIAEDEEWKRLRSLLSPTFTSGKLKEMVPIIAQYGDVLVRNLRREAETGKPVTLKDVFGAYSMDVITSTSFGVNIDSLNNPQDPFVENTKKLLRFDFLDPFFLSITVFPFLIPILEVLNICVFPREVTNFLRKSVKRMKESRLEDTQKHRVDFLQLMIDSQNSKETESHKALSDLELVAQSIIFIFAGYETTSSVLSFIMYELATHPDVQQKLQEEIDAVLPNKAPPTYDTVLQMEYLDMVVNETLRLFPIAMRLERVCKKDVEINGMFIPKGVVVMIPSYALHRDPKYWTEPEKFLPERFSKKNKDNIDPYIYTPFGSGPRNCIGMRFALMNMKLALIRVLQNFSFKPCKETQIPLKLSLGGLLQPEKPVVLKVESRDGTVSGA

SEQ ID NO: 5 - protein, 446 aa, Homo sapiens
  P450 isozyme CYP2D6_1
MGLEALVPLAVIVAIFLLLVDLMHRRQRWAARYPPGPLPLPGLGNLLHVDFQNTPYCFDQLRRRFGDVFSLQLAWTPVVVLNGLAAVREALVTHGEDTADRPPVPITQILGFGPRSQGRPFRPNGLLDKAVSNVIASLTCGRRFEYDDPRFLRLLDLAQEGLKEESGFLREVLNAVPVLLHIPALAGKVLRFQKAFLTQLDELLTEHRMTWDPAQPPRDLTEAFLAEMEKAKGNPESSFNDENLCIVVADLFSAGMVTTSTTLAWGLLLMILHPDVQRRVQQEIDDVIGQVRRPEMGDQAHMPYTTAVIHEVQRFGDIVPLGVTHMTSRDIEVQGFRIPKGTTLITNLSSVLKDEAVWEKPFRFHPEHFLDAQGHFVKPEAFLPFSAGRRACLGEPLARMELFLFFTSLLQHFSFSVPTGQPRPSHHGVFAFLVTPSPYELCAVPR

SEQ ID NO: 6 - protein, 490 aa, Homo sapiens
  P450 isozyme CYP2C19
MDPFVVLVLCLSCLLLLSIWRQSSGRGKLPPGPTPLPVIGNILQIDIKDVSKSLTNLSKIYGPVFTLYFGLERMVVLHGYEVVKEALIDLGEEFSGRGHFPLAERANRGFGIVFSNGKRWKEIRRFSLMTLRNFGMGKRSIEDRVQEEARCLVEELRKTKASPCDPTFILGCAPCNVICSIIFQKRFDYKDQQFLNLMEKLNENIRIVSTPWIQICNNFPTIIDYFPGTHNKLLKNLAFMESDILEKVKEHQESMDINNPRDFIDCFLIKMEKEKQNQQSEFTIENLVITAADLLGAGTETTSTTLRYALLLLLKHPEVTAKVQEEIERVIGRNRSPCMQDRGHMPYTDAVVHEVQRYIDLIPTSLPHAVTCDVKFRNYLIPKGTTILTSLTSVLHDNKEFPNPEMFDPRHFLDEGGNFKKSNYFMPFSAGKRICVGEGLARMELFLFLTFILQNFNLKSLIDPKDLDTTPVVNGFASVPPFYQLCFIPV

SEQ ID NO: 7 - protein, 516 aa, Homo sapiens
  P450 isozyme CYP1A2
MALSQSVPFSATELLLASAIFCLVFWVLKGLRPRVPKGLKSPPEPWGWPLLGHVLTLGKNPHLALSRMSQRYGDVLQIRIGSTPVLVLSRLDTIRQALVRQGDDFKGRPDLYTSTLITDGQSLTFSTDSGPVWAARRRLAQNALNTFSIASDPASSSSCYLEEHVSKEAKALISRLQELMAGPGHFDPYNQVVVSVANVIGAMCFGQHFPESSDEMLSLVKNTHEFVETASSGNPLDFFPILRYLPNPALQRFKAFNQRFLWFLQKTVQEHYQDFDKNSVRDITGALFKHSKKGPRASGNLIPQEKIVNLVNDIFGAGFDTVTTAISWSLMYLVTKPEIQRKIQKELDTVIGRERRPRLSDRPQLPYLEAFILETFRHSSFLPFTIPHSTTRDTTLNGFYIPKKCCVFVNQWQVNHDPELWEDPSEFRPERFLTADGTAINKPLSEKMMLFGMGKRRCIGEVLAKWEIFLFLAILLQQLEFSVPPGVKVDLTPIYGLTMKHARCEHVQARLRFSIN

SEQ ID NO: 8 - protein, 491 aa, Homo sapiens
  P450 isozyme CYP2B6
MELSVLLFLALLTGLLLLLVQRHPNTHDRLPPGPRPLPLLGNLLQMDRRGLLKSFLRFREKYGDVFTVHLGPRPVVMLCGVEAIREALVDKAEAFSGRGKIAMVDPFFRGYGVIFANGNRWKVLRRFSVTTMRDFGMGKRSVEERIQEEAQCLIEELRKSKGALMDPTFLFQSITANIICSIVFGKRFHYQDQEFLKMLNLFYQTFSLISSVFGQLFELFSGFLKYFPGAHRQVYKNLQEINAYIGHSVEKHRETLDPSAPKDLIDTYLLHMEKEKSNAHSEFSHQNLNLNTLSLFFAGTETTSTTLRYGFLLMLKYPHVAERVYREIEQVIGPHRPPELHDRAKMPYTEAVIYEIQRFSDLLPMGVPHIVTQHTSFRGYIIPKDTEVFLILSTALHDPHYFEKPDAFNPDHFLDANGALKKTEAFIPFSLGKRICLGEGIARAELFLFFTTILQNFSMASPVAPEDIDLTPQECGVGKIPPTYQIRFLPR

SEQ ID NO: 9 - protein, 401 aa, artificial sequence
  cytidine deaminase (CDA)-BFP ORF
MAQKRPACTLKPECVQQLLVCSQEAKKSAYCPYSHFPVGAALLTQEGRIFKGCNIENACYPLGICAERTAIQKAVSEGYKDFRAIAIASDMQDDFISPCGACRQVMREFGTNWPVYMTKPDGTYIVMTVQELLPSSFGPEDLQKTQGSEGRGSLLTCGDVEENPGPSGSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNE

SEQ ID NO: 10 - protein, 752 aa, artificial sequence
  CYP2D6_2-BFP ORF
MGLEALVPLAVIVAIFLLLVDLMHRRQRWAARYPPGPLPLPGLGNLLHVDFQNTPYCFDQLRRRFGDVFSLQLAWTPVVVLNGLAAVREALVTHGEDTADRPPVPITQILGFGPRSQGVFLARYGPAWREQRRFSVSTLRNLGLGKKSLEQWVTEEAACLCAAFANHSGRPFRPNGLLDKAVSNVIASLTCGRRFEYDDPRFLRLLDLAQEGLKEESGFLREVLNAVPVLLHIPALAGKVLRFQKAFLTQLDELLTEHRMTWDPAQPPRDLTEAFLAEMEKAKGNPESSFNDENLCIVVADLFSAGMVTTSTTLAWGLLLMILHPDVQRRVQQEIDDVIGQVRRPEMGDQAHMPYTTAVIHEVQRFGDIVPLGVTHMTSRDIEVQGFRIPKGTTLITNLSSVLKDEAVWEKPFRFHPEHFLDAQGHFVKPEAFLPFSAGRRACLGEPLARMELFLFFTSLLQHFSFSVPTGQPRPSHHGVFAFLVTPSPYELCAVPRGSEGRGSLLTCGDVEENPGPSGSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNE

SEQ ID NO: 11 - protein, 745 aa, artificial sequence
  CYP2C9-BFP ORF
MDSLVVLVLCLSCLLLLSLWRQSSGRGKLPPGPTPLPVIGNILQIGIKDISKSLTNLSKVYGPVFTLYFGLKPIVVLHGYEAVKEALIDLGEEFSGRGIFPLAERANRGFGIVFSNGKKWKEIRRFSLMTLRNFGMGKRSIEDRVQEEARCLVEELRKTKASPCDPTFILGCAPCNVICSIIFHKRFDYKDQQFLNLMEKLNENIKILSSPWIQICNNFSPIIDYFPGTHNKLLKNVAFMKSYILEKVKEHQESMDMNNPQDFIDCFLMKMEKEKHNQPSEFTIESLENTAVDLFGAGTETTSTTLRYALLLLLKHPEVTAKVQEEIERVIGRNRSPCMQDRSHMPYTDAVVHEVQRYIDLLPTSLPHAVTCDIKFRNYLIPKGTTILISLTSVLHDNKEFPNPEMFDPHHFLDEGGNFKKSKYFMPFSAGKRICVGEALAGMELFLFLTSILQNFNLKSLVDPKNLDTTPVVNGFASVPPFYQLCFIPVGSEGRGSLLTCGDVEENPGPSGSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNE

SEQ ID NO: 12 - protein, 758 aa, artificial sequence
  CYP3A4-BFP ORF
MALIPDLAMETWLLLAVSLVLLYLYGTHSHGLFKKLGIPGPTPLPFLGNILSYHKGFCMFDMECHKKYGKVWGFYDGQQPVLAITDPDMIKTVLVKECYSVFTNRRPFGPVGFMKSAISIAEDEEWKRLRSLLSPTFTSGKLKEMVPIIAQYGDVLVRNLRREAETGKPVTLKDVFGAYSMDVITSTSFGVNIDSLNNPQDPFVENTKKLLRFDFLDPFFLSITVFPFLIPILEVLNICVFPREVTNFLRKSVKRMKESRLEDTQKHRVDFLQLMIDSQNSKETESHKALSDLELVAQSIIFIFAGYETTSSVLSFIMYELATHPDVQQKLQEEIDAVLPNKAPPTYDTVLQMEYLDMVVNETLRLFPIAMRLERVCKKDVEINGMFIPKGVVVMIPSYALHRDPKYWTEPEKFLPERFSKKNKDNIDPYIYTPFGSGPRNCIGMRFALMNMKLALIRVLQNFSFKPCKETQIPLKLSLGGLLQPEKPVVLKVESRDGTVSGAGSEGRGSLLTCGDVEENPGPSGSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNE

SEQ ID NO: 13 - protein, 701 aa, artificial sequence
  CYP2D6_1-BFP ORF
MGLEALVPLAVIVAIFLLLVDLMHRRQRWAARYPPGPLPLPGLGNLLHVDFQNTPYCFDQLRRRFGDVFSLQLAWTPVVVLNGLAAVREALVTHGEDTADRPPVPITQILGFGPRSQGRPFRPNGLLDKAVSNVIASLTCGRRFEYDDPRFLRLLDLAQEGLKEESGFLREVLNAVPVLLHIPALAGKVLRFQKAFLTQLDELLTEHRMTWDPAQPPRDLTEAFLAEMEKAKGNPESSFNDENLCIVVADLFSAGMVTTSTTLAWGLLLMILHPDVQRRVQQEIDDVIGQVRRPEMGDQAHMPYTTAVIHEVQRFGDIVPLGVTHMTSRDIEVQGFRIPKGTTLITNLSSVLKDEAVWEKPFRFHPEHFLDAQGHFVKPEAFLPFSAGRRACLGEPLARMELFLFFTSLLQHFSFSVPTGQPRPSHHGVFAFLVTPSPYELCAVPRGSEGRGSLLTCGDVEENPGPSGSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNE

SEQ ID NO: 14 - protein, 745 aa, artificial sequence
  CYP2C19-BFP ORF
MDPFVVLVLCLSCLLLLSIWRQSSGRGKLPPGPTPLPVIGNILQIDIKDVSKSLTNLSKIYGPVFTLYFGLERMVVLHGYEVVKEALIDLGEEFSGRGHFPLAERANRGFGIVFSNGKRWKEIRRFSLMTLRNFGMGKRSIEDRVQEEARCLVEELRKTKASPCDPTFILGCAPCNVICSIIFQKRFDYKDQQFLNLMEKLNENIRIVSTPWIQICNNFPTIIDYFPGTHNKLLKNLAFMESDILEKVKEHQESMDINNPRDFIDCFLIKMEKEKQNQQSEFTIENLVITAADLLGAGTETTSTTLRYALLLLLKHPEVTAKVQEEIERVIGRNRSPCMQDRGHMPYTDAVVHEVQRYIDLIPTSLPHAVTCDVKFRNYLIPKGTTILTSLTSVLHDNKEFPNPEMFDPRHFLDEGGNFKKSNYFMPFSAGKRICVGEGLARMELFLFLTFILQNFNLKSLIDPKDLDTTPVVNGFASVPPFYQLCFIPVGSEGRGSLLTCGDVEENPGPSGSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNE

SEQ ID NO: 15 - protein, 771 aa, artificial sequence
  CYP1A2-BFP ORF
MALSQSVPFSATELLLASAIFCLVFWVLKGLRPRVPKGLKSPPEPWGWPLLGHVLTLGKNPHLALSRMSQRYGDVLQIRIGSTPVLVLSRLDTIRQALVRQGDDFKGRPDLYTSTLITDGQSLTFSTDSGPVWAARRRLAQNALNTFSIASDPASSSSCYLEEHVSKEAKALISRLQELMAGPGHFDPYNQVVVSVANVIGAMCFGQHFPESSDEMLSLVKNTHEFVETASSGNPLDFFPILRYLPNPALQRFKAFNQRFLWFLQKTVQEHYQDFDKNSVRDITGALFKHSKKGPRASGNLIPQEKIVNLVNDIFGAGFDTVTTAISWSLMYLVTKPEIQRKIQKELDTVIGRERRPRLSDRPQLPYLEAFILETFRHSSFLPFTIPHSTTRDTTLNGFYIPKKCCVFVNQWQVNHDPELWEDPSEFRPERFLTADGTAINKPLSEKMMLFGMGKRRCIGEVLAKWEIFLFLAILLQQLEFSVPPGVKVDLTPIYGLTMKHARCEHVQARLRFSINGSEGRGSLLTCGDVEENPGPSGSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNE

SEQ ID NO: 16 - protein, 746 aa, artificial sequence
  CYP2B6-BFP ORF
MELSVLLFLALLTGLLLLLVQRHPNTHDRLPPGPRPLPLLGNLLQMDRRGLLKSFLRFREKYGDVFTVHLGPRPVVMLCGVEAIREALVDKAEAFSGRGKIAMVDPFFRGYGVIFANGNRWKVLRRFSVTTMRDFGMGKRSVEERIQEEAQCLIEELRKSKGALMDPTFLFQSITANIICSIVFGKRFHYQDQEFLKMLNLFYQTFSLISSVFGQLFELFSGFLKYFPGAHRQVYKNLQEINAYIGHSVEKHRETLDPSAPKDLIDTYLLHMEKEKSNAHSEFSHQNLNLNTLSLFFAGTETTSTTLRYGFLLMLKYPHVAERVYREIEQVIGPHRPPELHDRAKMPYTEAVIYEIQRFSDLLPMGVPHIVTQHTSFRGYIIPKDTEVFLILSTALHDPHYFEKPDAFNPDHFLDANGALKKTEAFIPFSLGKRICLGEGIARAELFLFFTTILQNFSMASPVAPEDIDLTPQECGVGKIPPTYQIRFLPRGSEGRGSLLTCGDVEENPGPSGSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNE

SEQ ID NO: 17 - DNA, 49 bp, Homo sapiens
  Target dCK2
tggttcctgaacctgttgccagatggtgcaatgttcaaagtactcaaga

SEQ ID NO: 18 - DNA, 2814 bp, artificial sequence
  TALEN dCK2 LEFT
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 19 - DNA, 2814 bp, artificial sequence
  TALEN dCK2 RIGHT
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 20 - DNA, 957 bp, Homo sapiens
  cytidine deaminase (CDA)gene
caaaccatgggaggctcctctcctagaccctgcatcctgaaagctgcgtacctgagagcctgcggtctggctgcagggacacacccaaggggaggagctgcaatcgtgtctggggccccagcccaggctggccggagctcctgtttcccgctgctctgctgcctgcccggggtaccaacatggcccagaagcgtcctgcctgcaccctgaagcctgagtgtgtccagcagctgctggtttgctcccaggaggccaagaagtcagcctactgcccctacagtcactttcctgtgggggctgccctgctcacccaggaggggagaatcttcaaagggtgcaacatagaaaatgcctgctacccgctgggcatctgtgctgaacggaccgctatccagaaggccgtctcagaagggtacaaggatttcagggcaattgctatcgccagtgacatgcaagatgattttatctctccatgtggggcctgcaggcaagtcatgagagagtttggcaccaactggcccgtgtacatgaccaagccggatggtacgtatattgtcatgacggtccaggagctgctgccctcctcctttgggcctgaggacctgcagaagacccagtgacagccagagaatgcccactgcctgtaacagccacctggagaacttcataaagatgtctcacagccctggggacacctgcccagtgggccccagccctacagggactgggcaaagatgatgtttccagattacactccagcctgagtcagcacccctcctagcaacctgccttgggacttagaacaccgccgccccctgccccacctttcctttccttcctgtgggccctctttcaaagtccagcctagtctggactgcttccccatcagccttcccaaggttctatcctgttccgagcaacttttctaattataaacatcacagaacatcctggatc

SEQ ID NO: 21 - DNA, 957 bp, Homo sapiens; identical to SEQ ID NO: 20
  CYP2D6 isoform 1 gene

SEQ ID NO: 22 - DNA, 1861 bp, Homo sapiens
  CYP2C9 gene
gtcttaacaagaagagaaggcttcaatggattctcttgtggtccttgtgctctgtctctcatgtttgcttctcctttcactctggagacagagctctgggagaggaaaactccctcctggccccactcctctcccagtgattggaaatatcctacagataggtattaaggacatcagcaaatccttaaccaatctctcaaaggtctatggccctgtgttcactctgtattttggcctgaaacccatagtggtgctgcatggatatgaagcagtgaaggaagccctgattgatcttggagaggagttttctggaagaggcattttcccactggctgaaagagctaacagaggatttggaattgttttcagcaatggaaagaaatggaaggagatccggcgtttctccctcatgacgctgcggaattttgggatggggaagaggagcattgaggaccgtgttcaagaggaagcccgctgccttgtggaggagttgagaaaaaccaaggcctcaccctgtgatcccactttcatcctgggctgtgctccctgcaatgtgatctgctccattattttccataaacgttttgattataaagatcagcaatttcttaacttaatggaaaagttgaatgaaaacatcaagattttgagcagcccctggatccagatctgcaataatttttctcctatcattgattacttcccgggaactcacaacaaattacttaaaaacgttgcttttatgaaaagttatattttggaaaaagtaaaagaacaccaagaatcaatggacatgaacaaccctcaggactttattgattgcttcctgatgaaaatggagaaggaaaagcacaaccaaccatctgaatttactattgaaagcttggaaaacactgcagttgacttgtttggagctgggacagagacgacaagcacaaccctgagatatgctctccttctcctgctgaagcacccagaggtcacagctaaagtccaggaagagattgaacgtgtgattggcagaaaccggagcccctgcatgcaagacaggagccacatgccctacacagatgctgtggtgcacgaggtccagagatacattgaccttctccccaccagcctgccccatgcagtgacctgtgacattaaattcagaaactatctcattcccaagggcacaaccatattaatttccctgacttctgtgctacatgacaacaaagaatttcccaacccagagatgtttgaccctcatcactttctggatgaaggtggcaattttaagaaaagtaaatacttcatgcctttctcagcaggaaaacggatttgtgtgggagaagccctggccggcatggagctgtttttattcctgacctccattttacagaactttaacctgaaatctctggttgacccaaagaaccttgacaccactccagttgtcaatggatttgcctctgtgccgcccttctaccagctgtgcttcattcctgtctgaagaagagcagatggcctggctgctgctgtgcagtccctgcagctctctttcctctggggcattatccatctttcactatctgtaatgccttttctcacctgtcatctcacattttcccttccctgaagatctagtgaacattcgacctccattacggagagtttcctatgtttcactgtgcaaatatatctgctattctccatactctgtaacagttgcattgactgtcacataatgctcatacttatctaatgttgagttattaatatgttattattaaatagagaaatatgatttgtgtattataattcaaaggcatttcttttctgcatgttctaaataaaaagcattattatttgctga

SEQ ID NO: 23 - DNA, 2792 bp, Homo sapiens
  CYP3A4 gene
aatcactgctgtgcagggcaggaaagctccatgcacatagcccagcaaagagcaacacagagctgaaaggaagactcagaggagagagataagtaaggaaagtagtgatggctctcatcccagacttggccatggaaacctggcttctcctggctgtcagcctggtgctcctctatctatatggaacccattcacatggactttttaagaagcttggaattccagggcccacacctctgccttttttgggaaatattttgtcctaccataagggcttttgtatgtttgacatggaatgtcataaaaagtatggaaaagtgtggggcttttatgatggtcaacagcctgtgctggctatcacagatcctgacatgatcaaaacagtgctagtgaaagaatgttattctgtcttcacaaaccggaggccttttggtccagtgggatttatgaaaagtgccatctctatagctgaggatgaagaatggaagagattacgatcattgctgtctccaaccttcaccagtggaaaactcaaggagatggtccctatcattgcccagtatggagatgtgttggtgagaaatctgaggcgggaagcagagacaggcaagcctgtcaccttgaaagacgtctttggggcctacagcatggatgtgatcactagcacatcatttggagtgaacatcgactctctcaacaatccacaagacccctttgtggaaaacaccaagaagcttttaagatttgattttttggatccattctttctctcaataacagtctttccattcctcatcccaattcttgaagtattaaatatctgtgtgtttccaagagaagttacaaattttttaagaaaatctgtaaaaaggatgaaagaaagtcgcctcgaagatacacaaaagcaccgagtggatttccttcagctgatgattgactctcagaattcaaaagaaactgagtcccacaaagctctgtccgatctggagctcgtggcccaatcaattatctttatttttgctggctatgaaaccacgagcagtgttctctccttcattatgtatgaactggccactcaccctgatgtccagcagaaactgcaggaggaaattgatgcagttttacccaataaggcaccacccacctatgatactgtgctacagatggagtatcttgacatggtggtgaatgaaacgctcagattattcccaattgctatgagacttgagagggtctgcaaaaaagatgttgagatcaatgggatgttcattcccaaaggggtggtggtgatgattccaagctatgctcttcaccgtgacccaaagtactggacagagcctgagaagttcctccctgaaagattcagcaagaagaacaaggacaacatagatccttacatatacacaccctttggaagtggacccagaaactgcattggcatgaggtttgctctcatgaacatgaaacttgctctaatcagagtccttcagaacttctccttcaaaccttgtaaagaaacacagatccccctgaaattaagcttaggaggacttcttcaaccagaaaaacccgttgttctaaaggttgagtcaagggatggcaccgtaagtggagcctgaattttcctaaggacttctgctttgctcttcaagaaatctgtgcctgagaacaccagagacctcaaattactttgtgaatagaactctgaaatgaagatgggcttcatccaatggactgcataaataaccggggattctgtacatgcattgagctctctcattgtctgtgtagagtgttatacttgggaatataaaggaggtgaccaaatcagtgtgaggaggtagatttggctcctctgcttctcacgggactatttccaccacccccagttagcaccattaactcctcctgagctctgataagagaatcaacatttctcaataatttcctccacaaattattaatgaaaataagaattattttgatggctctaacaatgacatttatatcacatgttttctctggagtattctataagttttatgttaaatcaataaagaccactttacaaaagtattatcagatgctttcctgcacattaaggagaaatctatagaactgaatgagaaccaacaagtaaatatttttggtcattgtaatcactgttggcgtggggcctttgtcagaactagaatttgattattaacataggtgaaagttaatccactgtgactttgcccattgtttagaaagaatattcatagtttaattatgccttttttgatcaggcacagtggctcacgcctgtaatcctagcagtttgggaggctgagccgggtggatcgcctgaggtcaggagttcaagacaagcctggcctacatggttgaaaccccatctctactaaaaatacacaaattagctaggcatggtggactcgcctgtaatctcactacacaggaggctgaggcaggagaatcacttgaacctgggaggcggatgttgaagtgagctgagattgcaccactgcactccagtctgggtgagagtgagactcagtcttaaaaaaatatgcctttttgaagcacgtacattttgtaacaaagaactgaagctcttattatattattagttttgatttaatgttttcagcccatctcctttcatatttctgggagacagaaaacatgtttccctacacctcttgcattccatcctcaacacccaactgtctcgatgcaatgaacacttaataaaaaacagtcgattggtcaattgattgagcaataagcct

SEQ ID NO: 24 - DNA, 1658 bp, Homo sapiens
  CYP2D6 isoform 2 gene
gtgctgagagtgtcctgcctggtcctctgtgcctggtggggtgggggtgccaggtgtgtccagaggagcccatttggtagtgaggcaggtatggggctagaagcactggtgcccctggccgtgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggggtgttcctggcgcgctatgggcccgcgtggcgcgagcagaggcgcttctccgtgtccaccttgcgcaacttgggcctgggcaagaagtcgctggagcagtgggtgaccgaggaggccgcctgcctttgtgccgccttcgccaaccactccggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagcgccgtgtccaacaggagatcgacgacgtgatagggcaggtgcggcgaccagagatgggtgaccaggctcacatgccctacaccactgccgtgattcatgaggtgcagcgctttggggacatcgtccccctgggtgtgacccatatgacatcccgtgacatcgaagtacagggcttccgcatccctaagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctccctagccagaggctctaatgtacaataaagcaatgtggtagttcca

SEQ ID NO: 25 - DNA, 1787 bp, Homo sapiens
  CYP2C19 gene
gtcttaacaagaggagaaggcttcaatggatccttttgtggtccttgtgctctgtctctcatgtttgcttctcctttcaatctggagacagagctctgggagaggaaaactccctcctggccccactcctctcccagtgattggaaatatcctacagatagatattaaggatgtcagcaaatccttaaccaatctctcaaaaatctatggccctgtgttcactctgtattttggcctggaacgcatggtggtgctgcatggatatgaagtggtgaaggaagccctgattgatcttggagaggagttttctggaagaggccatttcccactggctgaaagagctaacagaggatttggaatcgttttcagcaatggaaagagatggaaggagatccggcgtttctccctcatgacgctgcggaattttgggatggggaagaggagcattgaggaccgtgttcaagaggaagcccgctgccttgtggaggagttgagaaaaaccaaggcttcaccctgtgatcccactttcatcctgggctgtgctccctgcaatgtgatctgctccattattttccagaaacgtttcgattataaagatcagcaatttcttaacttgatggaaaaattgaatgaaaacatcaggattgtaagcaccccctggatccagatatgcaataattttcccactatcattgattatttcccgggaacccataacaaattacttaaaaaccttgcttttatggaaagtgatattttggagaaagtaaaagaacaccaagaatcgatggacatcaacaaccctcgggactttattgattgcttcctgatcaaaatggagaaggaaaagcaaaaccaacagtctgaattcactattgaaaacttggtaatcactgcagctgacttacttggagctgggacagagacaacaagcacaaccctgagatatgctctccttctcctgctgaagcacccagaggtcacagctaaagtccaggaagagattgaacgtgtcattggcagaaaccggagcccctgcatgcaggacaggggccacatgccctacacagatgctgtggtgcacgaggtccagagatacatcgacctcatccccaccagcctgccccatgcagtgacctgtgacgttaaattcagaaactacctcattcccaagggcacaaccatattaacttccctcacttctgtgctacatgacaacaaagaatttcccaacccagagatgtttgaccctcgtcactttctggatgaaggtggaaattttaagaaaagtaactacttcatgcctttctcagcaggaaaacggatttgtgtgggagagggcctggcccgcatggagctgtttttattcctgaccttcattttacagaactttaacctgaaatctctgattgacccaaaggaccttgacacaactcctgttgtcaatggatttgcttctgtcccgcccttctatcagctgtgcttcattcctgtctgaagaagcacagatggtctggctgctcctgtgctgtccctgcagctctctttcctctggtccaaatttcactatctgtgatgcttcttctgacccgtcatctcacattttcccttcccccaagatctagtgaacattcagcctccattaaaaaagtttcactgtgcaaatatatctgctattccccatactctataatagttacattgagtgccacataatgctgatacttgtctaatgttgagttattaacatattattattaaatagagaaagatgatttgtgtattat

SEQ ID NO: 26 - DNA, 3127 bp, Homo sapiens
  CYP1A2 gene
gaagctccacaccagccattacaaccctgccaatctcaagcacctgcctctacagttggtacagatggcattgtcccagtctgttcccttctcggccacagagcttctcctggcctctgccatcttctgcctggtattctgggtgctcaagggtttgaggcctcgggtccccaaaggcctgaaaagtccaccagagccatggggctggcccttgctcgggcatgtgctgaccctggggaagaacccgcacctggcactgtcaaggatgagccagcgctacggggacgtcctgcagatccgcattggctccacgcccgtgctggtgctgagccgcctggacaccatccggcaggccctggtgcggcagggcgacgatttcaagggccggcctgacctctacacctccaccctcatcactgatggccagagcttgaccttcagcacagactctggaccggtgtgggctgcccgccggcgcctggcccagaatgccctcaacaccttctccatcgcctctgacccagcttcctcatcctcctgctacctggaggagcatgtgagcaaggaggctaaggccctgatcagcaggttgcaggagctgatggcagggcctgggcacttcgacccttacaatcaggtggtggtgtcagtggccaacgtcattggtgccatgtgcttcggacagcacttccctgagagtagcgatgagatgctcagcctcgtgaagaacactcatgagttcgtggagactgcctcctccgggaaccccctggacttcttccccatccttcgctacctgcctaaccctgccctgcagaggttcaaggccttcaaccagaggttcctgtggttcctgcagaaaacagtccaggagcactatcaggactttgacaagaacagtgtccgggacatcacgggtgccctgttcaagcacagcaagaaggggcctagagccagcggcaacctcatcccacaggagaagattgtcaaccttgtcaatgacatctttggagcaggatttgacacagtcaccacagccatctcctggagcctcatgtaccttgtgaccaagcctgagatacagaggaagatccagaaggagctggacactgtgattggcagggagcggcggccccggctctctgacagaccccagctgccctacttggaggccttcatcctggagaccttccgacactcctccttcttgcccttcaccatcccccacagcacaacaagggacacaacgctgaatggcttctacatccccaagaaatgctgtgtcttcgtaaaccagtggcaggtcaaccatgacccagagctgtgggaggacccctctgagttccggcctgagcggttcctcaccgccgatggcactgccattaacaagcccttgagtgagaagatgatgctgtttggcatgggcaagcgccggtgtatcggggaagtcctggccaagtgggagatcttcctcttcctggccatcctgctacagcaactggagttcagcgtgccgccgggcgtgaaagtcgacctgacccccatctacgggctgaccatgaagcacgcccgctgtgaacatgtccaggcgcggctgcgcttctccatcaattgaagaagacaccaccattctgaggccagggagcgagtgggggccagccacggggactcagcccttgtttctcttcctttctttttttaaaaaatagcagctttagccaagtgcagggcctgtaatcccagcattttaggaggccaaggttggaggatcatttgagcccaggaattggaaagcagcctggccaacatagtgggaccctgtctctacaaaaaaaaaatttgccaagagcctgagtgacagagcaagaccccatctcaaaaaaaaaaacaaacaaacaaaaaaaaaaccatatatatacatatatatatagcagctttatggagatataattcttatgccatataattcaccttctttttttttttttgtctgagacagaatctcagtctgtcacccaggttggagtgcagtggcgtgatctcagctcactgcaacctccacctcgcaggttcaagcaatcctcccacttcagcctcccaagcacctgggattacaagcatgagtcactacgcctggctgatttttgtagttttagtggagatggggtttcaccatgttggccaggcttgtctcgaactcctgaccccaagttatccacctgccttggcttcccaaagtcctgggattacaggtgtgagccaccacatccagcctaacttacattcttaaagtgtcgaatgacttctagtgtagaattgtgcaaccatcaccagaattaattttattattcttattatttttgagacagagtcttactctgttgccaggctggagtgcagtggcgcgatctcagctcactacaacctccgcctcccatgttcaagcgattctcctgcctcagcctcccgagtagctgggactataggcatgcgccaccatggccagctaatttttgtatttttagtagagacgaggtttcactgtgttggccaggatggtctccatctcttgacctcgtgatccacccgcctcagcctcccaaagtgctgggattaacaggtatgaaccaccgcgcccagcctttttgttttttttttttttgagacagagtcttcctctgtctcctaagctggagtgcagtggcatcatctcagctcactgcaacctctgcctcccaggttcaagtgcttctccagcctcagcctcccaagtagctgagactacaggcacacaccaccacgcctggctaatttttgtatttttagtagagacgggtttcaccatgttggctagactagtctcaaactcctgacctcaagtgatctgcccgcctcgacctctctcaaagtgctggcattacaggtgtgagccacggtgcccggcccacaattaattttagaacattttcatcacccctaaaagaaaccctgcacccattagcagtccctccacatttccccctagcctgcctcccctgcctcaccagccctggcaactgctaatctactttctgtgtctatggatttgccttctctaaacatttcatataaatggaattacacaatg

SEQ ID NO: 27 - DNA, 2603 bp, Homo sapiens
  CYP2B6 gene
gcggagcgcgcacgcgggaacccgcgctggaggcgggcgagggccgaggggcagctagggagcgcggcttgaggagggcggggccgccccgcaggcccgccagtgtcctcagctgcctccgcgcgccaaagtcaaaccccgacacccgccggcgggccggtgagctcactagctgacccggcaggtcaggatctggcttagcggcgccgcgagctccagtgcgcgcacccgtggccgcctcccagccctctttgccggacgagctctgggccgccacaagactaaggaatggccaccccgcccaagagaagctgcccgtctttctcagccagctctgaggggacccgcatcaagaaaatctccatcgaagggaacatcgctgcagggaagtcaacatttgtgaatatccttaaacaattgtgtgaagattgggaagtggttcctgaacctgttgccagatggtgcaatgttcaaagtactcaagatgaatttgaggaacttacaatgtctcagaaaaatggtgggaatgttcttcagatgatgtatgagaaacctgaacgatggtcttttaccttccaaacatatgcctgtctcagtcgaataagagctcagcttgcctctctgaatggcaagctcaaagatgcagagaaacctgtattattttttgaacgatctgtgtatagtgacaggtatatttttgcatctaatttgtatgaatctgaatgcatgaatgagacagagtggacaatttatcaagactggcatgactggatgaataaccaatttggccaaagccttgaattggatggaatcatttatcttcaagccactccagagacatgcttacatagaatatatttacggggaagaaatgaagagcaaggcattcctcttgaatatttagagaagcttcattataaacatgaaagctggctcctgcataggacactgaaaaccaacttcgattatcttcaagaggtgcctatcttaacactggatgttaatgaagactttaaagacaaatatgaaagtctggttgaaaaggtcaaagagtttttgagtactttgtgatcttgctgaagactacaggcagccaaatggttccagatacttcagctttgtgtatcttcgtaacttcatattaatataagtttctttagaaaacccaagtttttaatcgtttttgttttaaggaaaaaagatttttaaaatgaatcttatgcaaaactttttgaccagtttcttttcttttgttttttttttaaaaaagacatttaaagacaaagacattatttctcatagcaggaaatgtagaggtagatggttccagtatcagcatagtgactaaactacattataaaagatccagcttccttctgtcattcccctcttttgtcttcctcagcaggttggcttttttccctggtgcctctcacttcgttggtgaccagtttcttaaactgaaagctttaatgttacatagtaaatggtagtgtgtcctgtgtaaattagtgtacctattaaaagttgcaaagtggaattaaaggaatccctagaataaggattctgaagttttattttaaattattatcttcttaacagtttagtcccacctcttacttcctgcctcagtctgctttctctactgtctggattaattaggcagcctgctataaagttaaagtcacacatttctattttgcaaacactgtgattactctttgctttgtagtttgctttgctttgtagggttctgcttttaagtttttctctttttcagacaaattactgataaaaatgatattgctctatatgtaatatatcctgaaagcattattttttgttgaataggaaataaaattaatgaagacagaggctagaaagcatccattaattaatgagacacacttaactacttatctctaaaccatctatgtgaatatttgtaaaaataatgaatggactcatcttagttctgtatataaatatattttctttctagtttgtttagttaaggtgtgcagtgtttttcctgtgtattaaacctttccattttacgttttagaaaattttatgtattttaaaataaggggaagagtcattttcacttttaaactactatttttctttccaagtcatttttgtttttggtttcttattcaaagatgataatttagtggattaaccagtccagacgcactgatctttgcaaaggagacttaatttcaaatctgtaattaccatacataaactgtctcattatacgtatgcatttttttagtttgtttttgtttggtataaattaatttgttaattaaatatttcttaagtataaaccttatgaactacagtggagctacactcattgaaatgtaatttcagttctaaaaagatgtaataatcattttagaattaaaatttattctacttttaaataaattatgaatattaaaggtgaaaattgtataaattactttgattccattttaagtggagacatatttcagtgatttttagtaacctttaaaaatgtataatgacttttaaaatttgtagaattgaaaagacgctaataaaaatttattatttatttgtcatgactc